(19) United States
(12) Patent Application Publication
(10) Pub. No.: US 2022/0170009 A1
(43) Pub. Date: Jun. 2, 2022

(54) USE OF BIOLOGICAL RNA SCAFFOLDS WITH IN VITRO SELECTION TO GENERATE ROBUST SMALL MOLECULE BINDING APTAMERS FOR GENETICALLY ENCODABLE BIOSENSORS

(71) Applicant: The Regents of the University of Colorado, A Body Corporate, Denver, CO (US)

(72) Inventors: Robert T. Batey, Boulder, CO (US); Ely B. Porter, Boxborough, MA (US)

(21) Appl. No.: 17/541,068

(22) Filed: Dec. 2, 2021

Related U.S. Application Data
(63) Continuation of application No. 16/468,945, filed on Jun. 12, 2019, now abandoned.
(60) Provisional application No. 62/432,879, filed on Dec. 12, 2016.

(51) Int. Cl. C12N 15/10; C12N 15/115

(52) U.S. Cl. CPC C12N 15/1093; C12N 15/115; C12N 2330/31; C12N 2310/531; C12N 2310/16

(57) ABSTRACT

Provided herein are libraries of scaffolds derived from riboswitches and small ribozymes and their methods of use. The scaffolds of the invention yield aptamers that are easily identified and characterized by virtue of the structural scaffold. The nature of the scaffold predisposes these RNAs for coupling to readout domains to engineer biosensors that function in vitro and in vivo. Biosensors, synthetic RNA agents and synthetic DNA agents, and their methods of use, are also provided.

### RELATED APPLICATION

This application is a continuation of U.S. application Ser. No. 16/468,945, filed Jun. 12, 2019, which is a 371 of PCT/US2017/065526, filed Dec. 11, 2017, which claims the benefit of priority of U.S. Provisional Patent Application No. 62/432,879, filed on Dec. 12, 2016, the contents of each are hereby incorporated by reference herein in their entireties for all purposes.

### STATEMENT REGARDING FEDERALLY SPONSORED RESEARCH OR DEVELOPMENT

This invention was made with government support under grant number CMMI CHE1150834 awarded by the National Science Foundation. The government has certain rights in the invention.

### SEQUENCE LISTING

A Sequence Listing is provided herewith as a text file, called, “162027-49276_Sequence-Listing_ST25”, created on Aug. 20, 2019 and having a size of 37.5 kb. The contents of the text file are incorporated herein by reference in their entirety.

### BACKGROUND

Allosteric RNA devices are increasingly viewed as important tools capable of monitoring enzyme evolution, optimizing engineered metabolic pathways, facilitating discovery of novel genes, and regulators of nucleic acid based therapeutics. One bottleneck in the development of these platforms, however, is the availability of small molecule binding RNA aptamers that robustly function in the cellular environment. While aptamers can be raised against nearly any desired target by in vitro selection, many of these RNA-based aptamers cannot be easily integrated into devices or do not reliably function in a cellular context. Accordingly, there remains a need for aptamers and methods for developing aptamers.

### SUMMARY

A novel approach is described herein using scaffolds derived from riboswitches and small ribozymes. This approach, applied here to 5-hydroxytryptophan in an exemplary aspect, yields aptamers that are easily identified and characterized by virtue of the structural scaffold. The nature of the scaffold predisposes these RNAs for coupling to readout domains to engineer nucleic acid devices that function in vitro and in the cellular context.

In one aspect, a library of oligonucleotides is provided comprising a plurality of non-identical oligonucleotides is provided. Individual oligonucleotides of the library comprise a first sequence comprising a helix domain, a second sequence comprising a first hairpin domain, and a third sequence comprising a second hairpin domain, wherein the helix domain, first hairpin domain and second hairpin domain form an oligonucleotide junction containing a ligand-binding domain, and wherein the library comprises a plurality of non-identical ligand-binding domains.

In one embodiment, each helix domain independently is a fully complementary helix optionally comprising one or more destabilizing nucleotides selected from the group consisting of a mismatched base pair, a G⋅U wobble base pair and a bulge. In one embodiment, each helix domain is a fully complementary helix.

In one embodiment, each first hairpin domain independently comprises one or more destabilizing nucleotides selected from the group consisting of a mismatched base pair, a G⋅U wobble base pair and a bulge and/or each second hairpin domain independently comprises one or more destabilizing nucleotides selected from the group consisting of a mismatched base pair, a G⋅U wobble base pair and a bulge.

In one embodiment, the helix domain is at least 4 to 10 base-pairs in length, or at least 10 base-pairs in length.

In one embodiment, the oligonucleotides are oligoribonucleotides.

In one embodiment, the oligonucleotides individually comprise a sequence having a series of linked sequences according to Formula I: P1-J1/2-P2-L2-P2′-J2/3-P3-L3-P3′-J3/1-P1′ (I), wherein “-” represents a bond, P1 and P1′ form the helix, P2, L2 and P2′ form the first hairpin, P3, L3 and P3′ form the second hairpin and J1/2, J2/3 and J3/1 together form the oligonucleotide junction. In one embodiment, J2/3 comprises a T-loop motif. In one embodiment, the T-loop motif comprises the sequence UUGAA, optionally wherein the guanosine of the T-loop forms a Watson-Crick base-pair with a cytidine in J3/1.

In one embodiment, the helix domain has a first end and a second end, and the first end is proximal to the oligonucleotide junction, and the second end is linked to an oligonucleotide-based readout module. In one embodiment, the oligonucleotide-based readout module is a fluorogenic, e.g., a Broccoli fluorophore binding aptamer, or a switch-based readout module, e.g., a pbuE switch. In one embodiment, the oligonucleotide-based readout module is an oligoribonucleotide-based readout module.

In one embodiment, individual oligonucleotides have sequence correspondence to a Bacillus subtilis xpt-pbuX guanine riboswitch sequence comprising about 23 variable nucleotide residues within the oligonucleotide junction, or individual oligonucleotides have sequence correspondence to a Vibrio cholera Vc2 cyclic di-GMP riboswitch sequence comprising about 21 variable nucleotide residues within the oligonucleotide junction, or individual oligonucleotides have sequence correspondence to a Schistosoma mansoni hammerhead ribozyme sequence comprising about 21 variable nucleotide residues within the oligonucleotide junction.

In one embodiment, the oligonucleotide junction is an N-way junction, wherein N is two, three, four or five, or wherein N is two, or wherein N is three, or wherein N is four, or wherein N is five.

In one embodiment, the library comprises from about 4²¹ to about 4²³ non-identical members.

In another aspect, a library of oligonucleotides comprising a plurality of non-identical oligonucleotides is provided. Individual oligonucleotides of the library comprise a first sequence comprising a helix domain, a second sequence comprising a first hairpin domain, and a third sequence comprising a second hairpin domain, wherein the helix domain, the first hairpin domain and the second hairpin domain form an oligonucleotide junction containing a pre-selected ligand-binding domain, and wherein the library comprises a plurality of non-identical ligand-binding domains.

In one embodiment, each helix domain independently is a fully complementary helix optionally comprising one or more destabilizing nucleotides selected from the group consisting of a mismatched base pair, a G⋅U wobble base pair and a bulge. In one embodiment, each helix domain is a fully complementary helix.

In one embodiment, each first hairpin domain independently comprises one or more destabilizing nucleotides selected from the group consisting of a mismatched base pair, a G⋅U wobble base pair and a bulge and/or each second hairpin domain independently comprises one or more destabilizing nucleotides selected from the group consisting of a mismatched base pair, a G⋅U wobble base pair and a bulge.

In one embodiment, the helix domain is at least 4 to 10 base-pairs in length or at least 10 base-pairs in length.

In one embodiment, the oligonucleotides are oligoribonucleotides.

In one embodiment, individual oligonucleotides comprise a sequence having a series of linked sequences according to Formula I: P1-J1/2-P2-L2-P2′-J2/3-P3-L3-P3′-J3/1-P1′(I), wherein “-” represents a bond, P1 and P1′ form the helix, P2, L2 and P2′ form the first hairpin, P3, L3 and P3′ form the second hairpin, and J1/2, J2/3 and J3/1 together form the oligonucleotide junction. In one embodiment, J2/3 comprises a T-loop motif. In one embodiment, the T-loop motif comprises the sequence UUGAA, optionally wherein the guanosine of the T-loop forms a Watson-Crick base-pair with a cytidine in J3/1.

In one embodiment, the helix domain has a first end and a second end, and the first end is proximal to the oligonucleotide junction, and the second end is linked to an oligonucleotide-based readout module. In one embodiment, the oligonucleotide-based readout module is a fluorogenic, e.g., is a Broccoli fluorophore binding aptamer, or switch-based readout module, e.g., a pbuE switch. In one embodiment, the oligonucleotide-based readout module is an oligoribonucleotide-based readout module.

In one embodiment, individual oligonucleotides comprise sequences having sequence correspondence to a Bacillus subtilis xpt-pbuX guanine riboswitch sequence comprising about 23 variable nucleotide residues within the oligonucleotide junction, or individual oligonucleotides comprise sequences having sequence correspondence to a Vibrio cholera Vc2 cyclic di-GMP riboswitch sequence comprising about 21 variable nucleotide residues within the oligonucleotide junction, or individual oligonucleotides comprise sequences having sequence correspondence to a Schistosoma mansoni hammerhead ribozyme sequence comprising about 21 variable nucleotide residues within the oligonucleotide junction.

In one embodiment, the oligonucleotide junction is an N-way junction, wherein N is two, three, four or five, or wherein N is two, or wherein N is three, or wherein N is four, or wherein N is five.

In one embodiment, the preselected ligand-binding site comprises a binding site for a compound selected from the group consisting of an amino acid, a peptide, a nucleobase, a nucleoside, a nucleotide, a metal ion, a neurotransmitter, a hormone, an active pharmaceutical ingredient, and derivatives thereof. In one embodiment, the preselected ligand-binding site comprises a binding site for a ligand selected from the group consisting of an amino acid, a nucleobase, a nucleoside, a nucleotide, a neurotransmitter, a hormone, and derivatives thereof. In one embodiment, the preselected ligand-binding site comprises a binding site for at least one ligand selected from the group consisting of 5-hydroxy-L-tryptophan, L-tryptophan, serotonin, and 5-hydroxy-L-tryptophan-methylamide. In one embodiment, the ligand is at least one of 5-hydroxy-L-tryptophan or serotonin.

In still another aspect, a method of selecting a plurality of non-identical ligand-binding oligonucleotides is provided. The method includes a step of contacting a library of oligonucleotides comprising a plurality of oligonucleotides with a ligand under conditions suitable for ligand binding, wherein individual oligonucleotides comprise a first sequence comprising a helix domain, a second sequence comprising a first hairpin domain, and a third sequence comprising a second hairpin domain, wherein the helix domain, first hairpin domain and second hairpin domain form an oligonucleotide junction, and a step of partitioning the library of oligonucleotides in a spatially addressable such that the plurality of non-identical ligand-binding oligonucleotides is selected, wherein the oligonucleotides having the oligonucleotide junction further comprise a ligand-binding domain, and wherein the ligand-binding domains of the library of oligonucleotides comprise variable nucleotide residues, is selected.

In one embodiment, the method further comprises a step comprising competitively partitioning the library of oligonucleotides with a solution of free ligand between the step of contacting and the step of partitioning.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1A shows the GR scaffold. The GR scaffold is derived from the aptamer domain of the B. subtilis xpt-pbuX guanine riboswitch. The aptamer is comprised of three paired (P) regions connected by the joining (J) regions of the three-way junction that contains the guanine (Gua, magenta) binding site (dashed lines represent direct RNA-ligand interactions). Nucleotides in outlined cyan are those that were randomized for selection. The terminal loops of P2 and P3 (L2 and L3, green box) participate in a tertiary interaction that organizes the domain. Below is the three dimensional structure of the RNA (PDB ID 4FE5) with the same coloring scheme emphasizing the spatial relationship between the ligand binding site and the randomized nucleotides.

FIG. 1B shows the CDG scaffold. The secondary (top) and tertiary structure (bottom) of the CDG scaffold derived from the aptamer domain V. cholera Vc2 cyclic di-GMP riboswitch (PDB ID 3IWN). The labeling and coloring scheme are as described in FIG. 1A.

FIG. 1C shows the HH scaffold. The secondary (top) and tertiary structure (bottom) of the HH scaffold derived from the S. mansoni hammerhead ribozyme (PDB ID 3ZP8). The labeling and coloring scheme are as described in FIG. 1A.

FIG. 2A shows the chemical structure of 5-hydroxy-L-tryptophan.

FIG. 2B shows an unrooted phylogenetic tree representation of the distance matrix of sequences derived from round 7 of the GR-SSIII selection. Sequences are grouped into three main clusters, which are independently colored. The distance is expressed as the maximum likelihood estimate (MLE) of how many substitutions have occurred per site between two nodes of the tree (bar shown for scale).

FIG. 2C shows an unrooted phylogenetic tree representation of the distance matrix of sequences derived from round 7 of the GR-GsI selection. The four clusters from which representative sequences were analyzed are shown in independent colors (legend shown to right); black region represent unanalyzed clusters and regions of the tree.

FIG. 2D shows a covariation model of six observed clusters derived from the GR selections; colors are consistent with FIGS. 2B and 2C. The dashed lines correspond to the regions of the scaffold that were randomized and the line connecting L2 and L3 denote clusters where the sequences were maintained that would support the tertiary interaction.

FIG. 3A shows the outcome of Selective 2′-Hydroxyl Acylation analyzed by Primer Extension (“SHAPE”) chemical probing of three sequences (5HTP-I, -II, and -III) in comparison to the native xpt guanine riboswitch. For clarity, the regions of the gel corresponding to the J2/3 strand and L3 are shown (entire gel shown in Supplementary FIG. 4a). While all three RNAs reveal ligand-dependent decreases in chemical reactivity of the RNA backbone within or adjacent to J2/3, only 5HTP-II preserves a signature hotspot of reactivity in L3 that is indicative of the formation of its interaction with L2 in the xpt guanine riboswitch.

FIG. 3B is a quantitation of the ligand-dependent differential intensities of SHAPE probing of 5HTP-II in the presence and absence of 5HTP reveals that the majority of reactivity changes are localized to the junction. Enhancement of the L3 signal suggests coupling between ligand binding in the junction and tertiary structure formation.

FIG. 3C shows an isothermal titration calorimetric (ITC) analysis of binding of 5HTP to the 5HTP-II with (left) and without (right) the 5′- and 3′-amplification cassettes, demonstrating that these regions do not affect ligand binding.

FIG. 3D shows a crystal structure of the 5HTP-II aptamer in complex with 5-hydroxytryptophan (magenta). The green highlights the L2-L3 interaction of the parent scaffold (FIG. 1A) and cyan indicates the nucleotides that were randomized in the starting RNA library.

FIG. 3E shows an overlay of the quantified SHAPE reactivity data in panel (B) on the crystal structure emphasizing the relationship between ligand-dependent changes in the dynamics of the RNA backbone and the structure.

FIG. 4A shows the binding pocket of 5HTP in the 5HTP-II aptamer. The 5HTP-binding pocket within the three-way junction forms a set of hydrogen bonding interactions that engages every polar functional group in 5-hydroxytryptophan except for one oxygen atom in the carboxylate group that becomes an amide to immobilize the compound. In addition, the complex is stabilized by stacking interactions between the hydroxyindole ring of 5HTP and to adenine bases (A48 and A49) in J2/3.

FIG. 4B shows the binding pocket of 5HTP in the 5HTP-II aptamer. The core of the binding pocket in the 5HTP-II aptamer (green) is a T-loop that superimposes almost perfectly with the T-loops from tRNA^{Phe} (orange) and the thiamine pyrophosphate (TPP) riboswitch (cyan). In each of the three examples, the space between two purines at the T4 and T5 positions (T1-5 numbering indicates the nucleotide position within the T-loop motif) enables intercalation of an aromatic ring.

FIG. 5A shows that a 5HTP aptamer-based biosensor functions in E. coli. The wild-type 5HTP-II aptamer specifically activates the fluorescence of the Broccoli reporter in the presence of 5HTP. At t=0 minutes, 2 mM 5HTP was added to the media.

FIG. 5B shows that the wild-type 5HTP-II aptamer does not specifically activate the fluorescence of the Broccoli reporter in the presence of L-tryptophan. At t=0 minutes, 5 mM L-tryptophan was added to the media.

FIG. 5C shows that a single point mutation in the 5HTP-binding pocket of the 5HTP-II aptamer (A48U) also ablates fluorescence in the presence of the fluorophore. At t=0 minutes, 2 mM 5HTP was added to the media.

FIG. 5D shows single cell traces of fluorescence induction for the wild type 5HTP-II-Broccoli sensor in the presence of 5HTP. At t=0 minutes, 2 mM 5HTP was added to the media.

FIG. 5E shows single cell traces of fluorescence induction for the wild type 5HTP-II-Broccoli sensor in the presence of L-tryptophan. At t=0 minutes, 5 mM L-tryptophan was added to the media.

FIG. 5F shows single cell traces of fluorescence induction for the binding incompetent 5HTP-II A48U construct in the presence of 5HTP. At t=0 minutes, 2 mM 5HTP was added to the media.

FIG. 6A shows the secondary structure of an artificial 5HTP/serotonin “ON” riboswitch based upon 5HTP-IV aptamer. The 5HTP-IV aptamer is boxed in a dashed line, and the solid boxed nucleotides correspond nucleotides directly involved in alternative structure formation.

FIG. 6B shows quantified single-turnover transcription reactions of the riboswitch demonstrating robust antitermination upon addition of 5HTP, serotonin, or 5HTP-NHme. Gel images of transcription reactions are shown to the right, displaying the ligand-dependent transition from terminated (T) to read-through (RT) products. Similar titration with L-tryptophan failed to yield read-through transcription.

FIG. 7A shows an unrooted phylogenetic tree representation of the distance matrix of sequences derived from round 7 of the CDG selection using the GsI reverse transcriptase. The cluster from which the 5HTP aptamer was derived is highlighted in red. The distance is expressed as the maximum likelihood estimate (MLE) of how many substitutions have occurred per site between two nodes of the tree (bar shown for scale).

FIG. 7B shows a covariation model of the 5HTP-VII aptamer; the solid red line corresponds to the regions of the bioscaffold that were randomized and the line connecting L2 and P3 denote the tertiary interaction.

FIG. 7C shows an unrooted phylogenetic tree representation of the distance matrix of sequences derived from round 7 of the HH selection using the GsI reverse transcriptase. The cluster from which the 5HTP-VIII aptamer was derived is highlighted in purple; black regions represent unanalyzed clusters and regions of the tree.

FIG. 7D shows a covariation model of the 5HTP-VIII aptamer; the solid purple line corresponds to the regions of the bioscaffold that were randomized and the line connecting P2 and L3 denote the tertiary interaction.

FIG. 8A shows a significant accumulation of mutations in the bioscaffold by round 7 of the selection with some positions at the 3′ end achieving greater than 90% mutation frequency in the initial selection using SuperScript III (Life Technologies). There is also a strong propensity for the accumulation of mutations in sequence elements critical for secondary and tertiary structure (P2 and P3).

FIG. 8B shows the modified selection protocol using a developed group II intron RT (GsI-IIC) shows a relief in the amount of accumulated mutations in the GR bioscaffold, particularly in the P2 and P3 regions. This allows for the preservation of structural elements designed into the sequence.

FIG. 8C shows the observed error frequencies as a function of nucleotide position in round 7 of the CDG/GsI selection.

FIG. 8D shows the observed error frequencies as a function of nucleotide position in round 7 of the HH/GsI selection.

FIG. 9A shows a SHAPE analysis of the 5HTP-IV, -V and -VI aptamers in the absence and presence of 5HTP. Bars to the side highlight the J2/3 and L3 regions demonstrating varying ligand-dependent protections in the three-way junction and the presence of the signature reactivity hotspot in L3 diagnostic of the L2-L3 interaction.

FIG. 9B shows a SHAPE analysis of a CDG scaffolded 5HTP binding aptamer. The raw gel shows clear ligand dependent modifications in J1/2 and J2/3. The parental Vc2 RNA shows a ligand dependent protection in P3 at the tetra-loop docking site, while the 5HTP-VII aptamer shows a ligand dependent modification on the opposite side of the helix. Additionally, neither RNA shows any modifications in the presence of the irrespective ligand.

FIG. 9C shows a SHAPE analysis of a HH scaffolded 5HTP binding aptamer. The raw gel shows clear ligand dependent modifications in J1/2 and J2/3. The changes in J2/3 localize mainly to positions 3 and 4 of a predicted T-loop motif. Additionally, if the structure was maintained the terminal loop of P3 (L3) that docks into P2 in the parental RNA shows a ligand dependent protection. Integration of the bands as a function of distance down the gel is shown on the left.

FIG. 10A shows the 2Fₒ-F_{c} electron density map of the 5HTP-II/5HTP complex around the model contoured at 2σ. All regions of the RNA are well-defined by the electron density, making placement of the residues and backbone unambiguous. Cyan nucleotides were randomized in the original RNA library and 5HTP is shown in orange.

FIG. 10B shows a composite omit of the ligand binding pocket of the 5HTP-II/5HTP complex contoured at 1σ showing clear density supporting placement of the ligand (5HTP) and adjacent iridium hexammine (IrHex).

FIG. 10C shows the final 2Fₒ-F_{c} electron density map of the 5HTP binding pocket of the 5HTP-II/5HTP complex contoured at 1σ.

FIG. 11A shows an R2R diagram derived from variation analysis of J2/3 of the 5HTP-VIII aptamer of the most populous cluster of the HH/GsI selection.

FIG. 11B shows a variation analysis of J2/3 of the 5HTP-VIII aptamer comparing the variation pattern of T-loops found in biological RNAs¹ (top) and the cluster containing the 5HTP-VIII aptamer.

FIG. 12 shows the construction scheme for 5HTP-Broccoli biosensors. Red nucleotides in the Broccoli secondary structure denote the G-quartet that forms the platform for DFHBI and green nucleotides denote the differences between Spinach and Broccoli.

FIG. 13 is a graphical abstract showing the design of novel scaffold aptamers of the invention.

FIG. 14A is a schematic of the secondary structure of genetically encodable biosensors of 5HTP and L-DOPA in which a GR-scaffolded aptamer (cyan) is coupled to a fluorogenic aptamer (Broccoli, green) via a communication module (orange, CM; sequences on bottom) and stabilized in vivo with the tRNA scaffold (yellow).

FIG. 14B and FIG. 14C depict heat maps of the observed ligand-induced fluorescence (top) and brightness of the ligand-bound sensor relative to a tRNA/Broccoli control (bottom) for a series of GR-scaffolded aptamers coupled to Broccoli with CMs of 2 to 5 base pairs.

FIG. 14D and FIG. 14E depict heat maps of the performance of the same sensors in E. coli.

FIG. 15A and FIG. 15B show that the wild-type 5GR-II aptamer specifically activates the fluorescence of the Broccoli reporter in the presence of 5HTP, but not in the presence of L-tryptophan.

FIG. 15C shows that a single point mutation in the 5HTP-binding pocket of the 5GR-II aptamer (A48U) also ablates fluorescence in the presence of the fluorophore.

FIG. 15D, FIG. 15E and FIG. 15F depict single-cell traces of fluorescence induction for the wild type 5GR-II-Broccoli sensor in the presence of 5HTP, L-tryptophan, and the binding incompetent 5GR-II A48U construct in the presence of 5HTP. At t=0 minutes, either 2 mM 5HTP or 5 mM L-tryptophan was added to the media.

FIG. 16A shows superimposition of parental B. subtillis xpt guanine riboswitch and 5GR-11 RNAs over all backbone atoms. The guanine riboswitch (PDB 4FE5) is shown in red and its ligand, hypoxanthine, shown in magenta. The 5GR-II aptamer is shown in blue and its ligand, 5HTP, shown in green.

FIG. 16B shows superimposition of the two RNAs using backbone atoms in P2 and P3 only.

FIG. 16C depicts a view of the superimposition of two base quadruples that comprise the core of the L2-L3 interaction, showing a complete preservation of the individual base interactions that establish this tertiary interaction.

FIG. 17A-FIG. 17D depicts sequence and secondary structure of initial RNA libraries. The green box highlights the constant region that is used for priming and the yellow box highlights the barcode specific to each scaffold. Nucleotide positions that were randomized in the starting library are highlighted in cyan.

FIG. 17A depicts the sequence of the guanine riboswitch aptamer (GR) RNA library for the selection using the SuperScript III RT.

FIG. 17B depicts the sequence of the GR RNA library used for the selection using the GsI-IIC RT.

FIG. 17C depicts the sequence of the di-cyclic GMP riboswitch aptamer (CG) library. FIG. 17D depicts the sequence of the hammerhead ribozyme (HR) library.

FIG. 18A-FIG. 18C depict selection of scaffolded aptamers that selectively bind 3,4-dihydroxyphenylalanine (L-DOPA).

FIG. 18A depicts chemical structure of dopamine (1) and L-DOPA (2).

FIG. 18B depicts an unrooted phylogenetic tree representation of the distance matrix of sequences derived from round 7 of a GR-GsI-IIC selection against L-DOPA. The four clusters from which representative sequences were incorporated into fluorogenic biosensors are shown in independent colors. The black region represents unanalyzed clusters and regions of the tree.

FIG. 18C depicts covariation models of the four clusters, with colors consistent with those of panel (B). Note that the DGR-III MFE structure has an alternative secondary structure that, if correct, ablates the tertiary loop-loop interaction.

FIG. 19 depicts SHAPE analysis of GR scaffolded 5HTP binding aptamers. This Figure shows the full sequencing region of the gel that was used to generate FIG. 4A.

FIG. 20 depicts SHAPE analysis of GR-scaffolded 5HTP-binding aptamers. The complete and unaltered image of the sequencing gel shown in FIG. 4A (regions corresponding to J2/3 and L3 were cropped to produce FIG. 4A). SHAPE analysis of the 5GR-IV, -V and -VI aptamers are depicted in the absence and presence of 5HTP. Bars to the side highlight the J2/3 and L3 regions demonstrating varying ligand-dependent protections in the 3WJ and the presence of the signature reactivity hotspot in L3 diagnostic of the L2-L3 interaction.

FIG. 21 depicts SHAPE analysis of a CG-scaffolded 5HTP-binding aptamer. The raw gel (inset) shows clear ligand dependent modifications in J1/2 and J2/3. The parental Vc2 RNA shows a ligand dependent protection in P3 at the tetra-loop docking site, while the 5CG-I aptamer shows a ligand dependent modification on the opposite side of the helix. Additionally, neither RNA shows modifications in the presence of the irrespective ligand. Integration of the bands as a function of distance down the gel is shown at the bottom. After normalization and assignment, the ligand dependent changes are still evident (colored asterisks), particularly in J1/2.

FIG. 22 depicts SHAPE analysis of a HR-scaffolded 5HTP-binding aptamer. The raw gel (right) shows clear ligand dependent modifications in J1/2 and J2/3. The changes in J2/3 localize mainly to positions 3 and 4 of a predicted T-loop motif. Additionally, if the structure was maintained, the terminal loop of P3 (L3) that docks into P2 in the parental RNA shows ligand-dependent protection. Integration of the bands as a function of distance down the gel is shown on the left. After normalization and assignment, the ligand-dependent changes are still evident (colored asterisks). Note that there is no background cleavage at the equivalent site in the parental hammerhead RNA (top green asterisk).

FIG. 23 depicts engineered sensors of the invention that were synthesized as G-blocks. “N” represents a position where the composition of A, C, G and T is approximately 25% each. RNA aptamer and sensor sequences are given as their equivalent DNA sequences. Separate domains of Broccoli sensors are color coded to denote the tRNA scaffold (grey), DFHBI-1T binding Broccoli aptamer (yellow), communication module (cyan) and GR scaffolded aptamer (red).

### DETAILED DESCRIPTION

The means to generate synthetic RNA and/or DNA elements with novel regulatory and sensing abilities is powerfully enabled by in vitro selection and the pool of available synthetic aptamers is currently large. However, only a few small molecule binding RNA aptamers have transitioned into effective and widely used intracellular biosensors of their cognate ligand or other RNA devices. This discrepancy between in vitro binding and intracellular activity is problematic, suggesting current selection strategies cannot easily access small molecule binding RNA aptamers capable of functioning robustly in the cellular environment. While in vivo selection strategies for small molecule binding RNAs might be more successful at generating cell-capable aptamers, these approaches are not currently broadly practical. Thus, current strategies continue to rely upon a protracted workflow incorporating traditional in vitro selection with tandem, application-specific selections for enhanced function.

Unlike synthetic aptamers, the small molecule binding domains of natural riboswitches have evolved in the context of the cell and incorporate additional features extending beyond the ligand binding site that include high fidelity folding and an ability to communicate with downstream regulatory switches to yield a detectable output. These aptamers are highly modular and robust, observed in a broad spectrum of bacterial species and interface with diverse regulatory domains acting on transcription, translation, alternative splicing and mRNA stability. Thus, they are highly flexible with regards to mechanisms of communication with adjacent domains or sequences that elicit an output (e.g., gene regulation). These aptamers have been successful in synthetic applications and have been used to validate synthetic RNA tools. While there has been a substantial effort to identify and characterize natural aptamers, they are inherently limited in their diversity and in application due to the endogenous pools of effector ligand that are difficult to modulate.

In response to these difficulties, provided herein are recurrent architectural folds found in natural RNA aptamers and small nulceolytic ribozymes that can be reprogrammed using in vitro selection to host a broad spectrum of small molecule binding sites while preserving the robust folding and highly stable architectural properties of the parent and their methods of use.

Using partially structured RNA libraries in the selection of small molecule binding aptamers has been previously employed, but these simple hairpins and helices do not have the potential to form higher-order structure akin to natural aptamers. Selection of an RNA ligase ribozyme of very modest activity by randomizing a terminal loop in the Tetrahymena ribozyme P456 domain demonstrated that it is possible to obtain ribozymes for a scaffolded library. However, the P456 architecture is large (160 nucleotides) and restricted to the IC1 and IC2 subclasses of group I self-splicing introns. Thus, it may not be well suited as a general platform for raising diverse small molecule binding aptamers that are active in a broad spectrum of cellular environments.

Using scaffolds derived from two different riboswitch aptamer domains and a ribozyme, a diverse set of aptamers was obtained that selectively binds 5-hydroxytryptophan (5HTP) and/or serotonin (5HT). While each of the scaffolds provides unique solutions for recognition, they all converge on similar binding affinities and discriminate against chemically related L-tryptophan. These aptamers are predisposed by the structural scaffold for coupling to fluorogenic and switch-based readout modules. While screening strategies are met with varying degrees of success, the diversity of aptamers readily achieved using this approach enables more flexible strategies with less in vitro characterization required to implement practical RNA devices.

RNA-based devices are increasingly viewed as a potentially robust and predictable tool in synthetic biology. RNA has a unique feature set when compared to protein-based alternatives, including the ability to regulate in cis, predictable secondary structure, and a small genetic footprint. Among the more sought after abilities of RNA devices are the capacity to sense external stimuli and modulate a genetic or phenotypic response in the absence of additional protein factors. Efforts have focused on creating synthetic riboswitches, aptazymes and fluorogenic RNA sensors, yet their potential has yet to be fully realized in part due to the limited availability of RNA sensing domains. The compositions and methods provided herein surprisingly demonstrate that using naturally evolved riboswitches or ribozymes as scaffolds for selection can produce a robust sensing domain capable of functioning both in vitro and in the cellular context, on par with the best artificial and natural aptamers to date.

A key strength of the compositions and methods described herein is the use of multiple scaffolds in parallel selections to obtain a suite of aptamers. While the aptamers derived from different scaffolds have similar affinities for 5HTP and selectivity against L-tryptophan, they clearly have distinct characteristics with respect to their ability to communicate with a readout domain via the P1 helix, a common feature to all of the scaffolds. Without being bound by theory, it is hypothesized that this is due to variation in the spatial relationship between the ligand and the interdomain (P1) helix, a feature that cannot be fully controlled in the selection. In biological riboswitches, the ligand is either in direct contact or induces conformational changes in the RNA that involve the P1 helix that links the aptamer to the downstream regulatory switch.

With a suite of aptamers, combinatorial approaches can be employed to rapidly screen for the sensors with desired properties without the extensive aptamer characterization or the device optimization. Typically, the traditional in vitro selection only yields a single small molecule binding aptamer, and development of an RNA device requires screening many communication modules and adaptor sequences while leaving the sensory aptamer as a fixed node. With the scaffolded selection approach, a set of distinct aptamers can be combinatorially coupled to a set of communication modules and rapidly screened for variants with the desired activity. In this fashion, this approach should eliminate the key bottleneck in the development of RNA devices and sensors. Notably, while in this study only the most populous clusters were focused on in each selection for characterization and sensor design, within each selection there were many clusters containing alternative sequences that could further enrich the initial pool of aptamers for developing downstream applications.

A second powerful advantage of this selection strategy is the robust folding in the cellular context provided by the tertiary interaction of the oligonucleotide junction architecture (e.g., the three-way junction). Each of these aptamers has a fold that has undergone extensive biological evolution and, in particular, the distal tertiary interactions that organize the three-way junction core are highly stable. Both the L2-L3 interaction of purine riboswitch and the tetraloop-tetraloop receptor of the cyclic di-GMP riboswitch scaffold are capable of stably forming outside of the context of other RNA structure. This enables these elements to potentially guide the folding of all members of the initial library such that the vast majority of the population contains the prescribed secondary and tertiary structure. Misfolding is often a significant problem for traditional synthetic aptamers, which can be greatly exacerbated when the RNA element is coupled to another or placed in the context of a larger RNA. Since there is no significant selection pressure for high fidelity folding in a typical selection protocol, providing this information in the starting library can be a path towards robust folding RNAs.

While the three-way junction scaffolds is exemplified herein, the diversity of natural riboswitches and ribozymes can provide further feedstock for this approach. Within the three-way junction family, there is a broad array of sequences that vary the orientation of the three helices, size of the joining regions, and the nature of the distal tertiary interaction that may provide superior scaffolds for a particular ligand or sensor. Furthermore, other folds may be predisposed to bind a target small molecule based on the nature of the cognate ligand. For example, another choice for a scaffold to bind 5HTP is the lysine riboswitch aptamer domain that contains a five-way junction that houses the ligand binding site and positions it adjacent to the P1 helix. Larger ligands may be more easily accommodated by flavin mononucleotide or cobalamin riboswitch derived scaffolds, while dinucleotides such as NADH may be readily accommodated by one of the other di-cyclic nucleotide aptamers. Thus, the scaffolded selection approaches described herein have the potential to facilitate the development of powerful new tools for monitoring and responding to small molecules in the cellular environment across a broad range of applications using RNA devices.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated.

Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

The terms “aptamer” and “aptamer domain” refer to short, single-stranded DNA, RNA or peptide sequences that specifically bind to various molecular targets such as small molecules, proteins, nucleic acids, cells, tissues and the like with high specificity and affinity. Aptamers are generally highly specific, relatively small in size, and non-immunogenic. Similar to antibodies, aptamers interact with their targets by recognizing a specific three-dimensional structure and are thus also known as “chemical antibodies.” In contrast to protein antibodies, DNA or RNA aptamers offer unique chemical and biological characteristics based on their oligonucleotide properties.

The term “riboswitch” refers to an element commonly found in the 5′-untranslated region of mRNAs that exerts its regulatory control over the transcript in a cis-fashion by directly binding a small molecule ligand. The typical riboswitch contains two distinct functional domains: an aptamer domain, which adopts a compact three-dimensional fold to scaffold the ligand binding pocket; and an expression platform, which contains a secondary structural switch that interfaces with the transcriptional or translational machinery. Regulation is achieved by virtue of a region of overlap between these two domains, known as the switching sequence, whose pairing directs folding of the RNA into one of two mutually exclusive structures in the expression platform that represent the on and off states of the mRNA. In certain exemplary embodiments, a preferred riboswitch is the B. subtilis xpt-pbuX guanine riboswitch (referred to herein as “GR”) or the Vibrio cholerae Vc2 cyclic di-GMP riboswitch (referred to herein as “CDG”).

The term “ribozyme” refers to an RNA molecule that acts as an enzyme and is capable of catalyzing specific biochemical reactions, similar to the action of protein enzymes. Ribozyme classes include GIR1 branching ribozyme, glmS ribozyme, Group I self-splicing intron, Group II self-splicing intron, hairpin ribozyme, hammerhead ribozyme and HDV ribozyme. In certain exemplary embodiments, a preferred ribozyme is Schistosoma mansoni hammerhead ribozyme (referred to herein as “HH”).

The terms “synthetic RNA agent,” “synthetic DNA agent,” “biosensor” and “scaffold” refer to nucleic acid sensory devices described herein that comprise secondary and tertiary structural scaffolds derived from aptamers that exist in nature, e.g., from riboswitches and ribozymes. A “synthetic RNA agent,” “synthetic DNA agent,” “biosensor” or “structural scaffold” of the invention includes a helix domain, first and second hairpin domains, and an oligonucleotide junction that contains a ligand-binding domain. A biosensor of the invention can comprise an N-way junction, wherein N is 2, 3, 4 or 5.

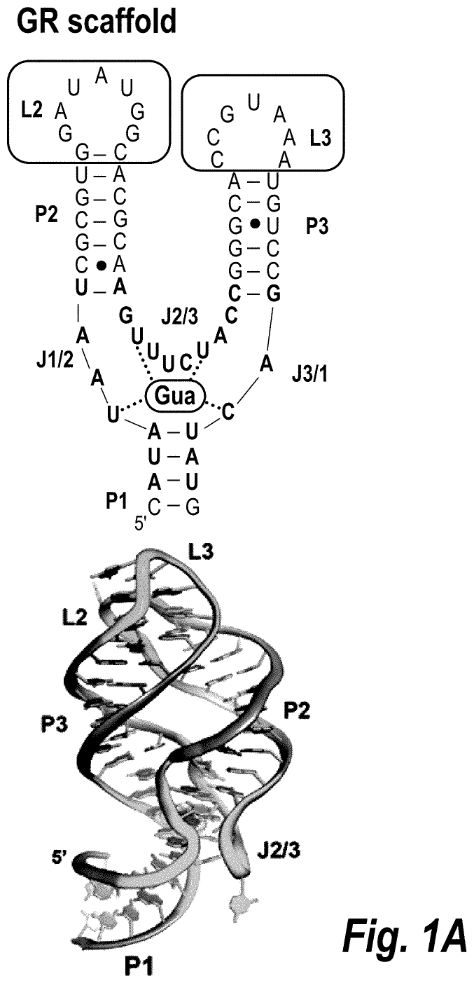

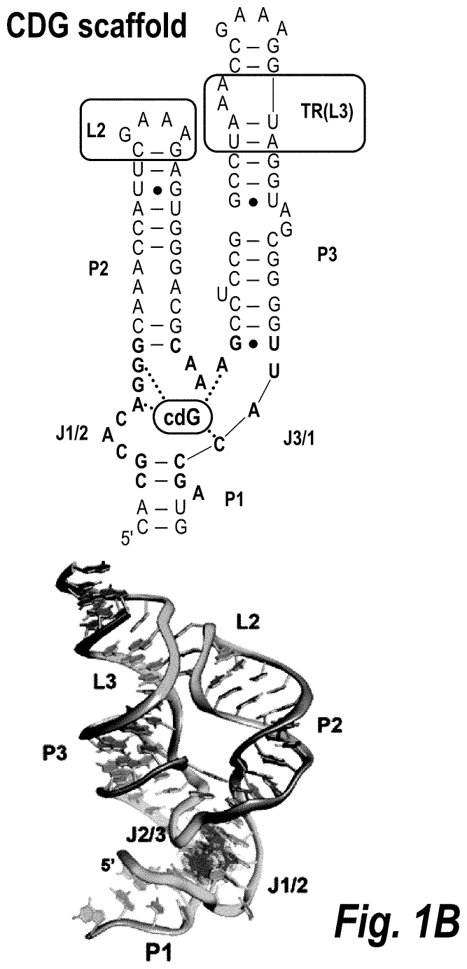

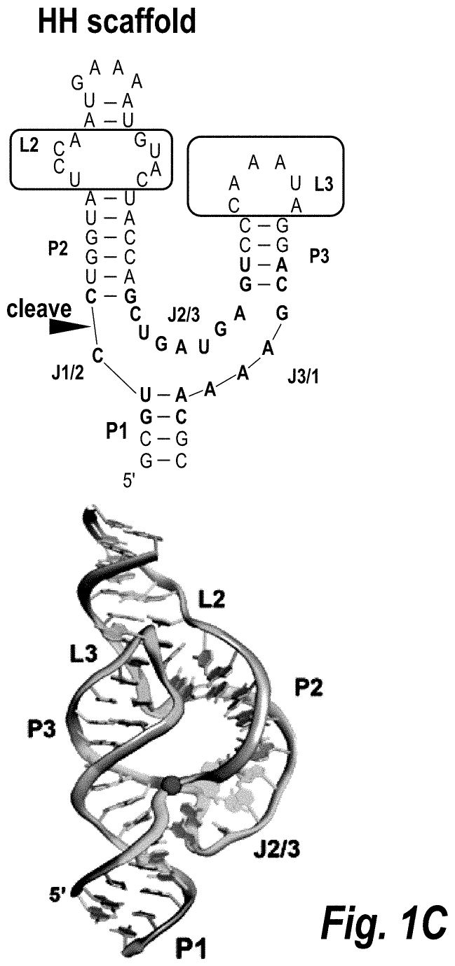

In certain embodiments, a biosensor of the invention comprises a sequence having a series of linked components according to Formula I: (I) P1-J1/2-P2-L2-P2′-J2/3-P3-L3-P3′-J3/1-, wherein “-” represents a bond, “P1” and “P1”' form the helix, “P2,” “L2” and “P2′” form the first hairpin, “P3,” “L3” and “P3′” form the second hairpin, and “J1/2,” “J2/3” and “J3/1” together form the oligonucleotide junction. (See, e.g., FIG. 1.)

In certain embodiments, a biosensor of the invention comprises a “read-out” module by which the specificity and/or affinity of a module for a ligand can be determined. A “read-out” can be visually detectable, e.g., a fluorogenic read-out, such as, e.g. Broccoli, or can be a riboswitch-based read-out or an oligonucleotide-based readout, as described further herein.

The term “helix domain” refers to two or more polynucleotides that are held together, e.g., by hydrogen, Hoogsteen or reversed Hoogsteen bonds, thus forming a double helix or a triple helix structure.

The term “hairpin domain” refers to the ability of a polynucleotide to base pair with itself such that the 5′ end and the 3′ end of the polynucleotide are brought into proximity to one another and are linked by a non-hybridizing portion of the polynucleotide that forms a loop structure.

The term “oligonucleotide junction” refers to two, three, four or five regions of a scaffold that form a ligand binding site, e.g., a Gua binding site. (See, e.g., FIG. 1.)

The term “oligonucleotide library” refers to a collection of synthetic oligonucleotide sequences, each sequence comprising a structural scaffold of the invention, wherein each structural scaffold includes at least a helix domain, first and second hairpin domains, and an oligonucleotide junction that contains a ligand-binding domain.

In certain exemplary embodiments, assays for screening ligands or test compounds which bind to a biosensor of the invention are provided. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 August 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 October 10(5):333-45, Stein, Antisense Nucleic Acid Drug Dev. 2001 October 11(5): 317-25, Vorobjev et al. Antisense Nucleic Acid Drug Dev. 2001 April 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

In certain exemplary embodiments, a detectable label can be used to detect one or more oligonucleotides and/or polynucleotides described herein. Examples of detectable markers include various radioactive moieties, enzymes, prosthetic groups, fluorescent markers, luminescent markers, bioluminescent markers, metal particles, protein-protein binding pairs, protein-antibody binding pairs and the like. Examples of fluorescent proteins include, but are not limited to, yellow fluorescent protein (YFP), green fluorescent protein (GFP), cyan fluorescent protein (CFP), umbelliferone, fluorescein, fluorescein isothiocyanate, rhodamine, dichlorotriazinylamine fluorescein, dansyl chloride, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as ¹²⁵I, ³⁵S, ¹⁴C or ³H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor,

Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat.

No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labelling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

The term “oligonucleotide” refers to a short polymer of nucleotides and/or nucleotide analogs. The term “RNA analog” or “DNA analogue” refers to a polynucleotide (e.g., a chemically synthesized polynucleotide) having at least one altered or modified nucleotide as compared to a corresponding unaltered or unmodified DNA or RNA but retaining the same or similar nature or function as the corresponding unaltered or unmodified DNA or RNA. As discussed above, the oligonucleotides may be linked with linkages which result in a lower rate of hydrolysis of the RNA or DNA analog as compared to an RNA or DNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Preferred RNA or DNA analogues include sugar- and/or backbone-modified ribonucleotides and/or deoxyribonucleotides. Such alterations or modifications can further include addition of non-nucleotide material, such as to the end(s) of the RNA or DNA or internally (at one or more nucleotides of the RNA or DNA).

As used herein, the term “isolated RNA” or “isolated DNA” refers to RNA or DNA molecules which are substantially free of other cellular material, or culture medium when produced by recombinant techniques, or substantially free of chemical precursors or other chemicals when chemically synthesized.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered

RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence.

A gene “involved” in a disease or disorder includes a gene, the normal or aberrant expression or function of which effects or causes the disease or disorder or at least one symptom of said disease or disorder.

As used herein, the term “sample population” refers to a population of individuals comprising a statistically significant number of individuals. For example, the sample population may comprise 50, 75, 100, 200, 500, 1000 or more individuals. In particular embodiments, the sample population may comprise individuals which share at least on common disease phenotype (e.g., a gain-of-function disorder) or mutation (e.g., a gain-of-function mutation).

As used herein, the term “heterozygosity” refers to the fraction of individuals within a population that are heterozygous (e.g., contain two or more different alleles) at a particular locus (e.g., at a SNP). Heterozygosity may be calculated for a sample population using methods that are well known to those skilled in the art.

The phrase “examining the function of a gene in a cell or organism” refers to examining or studying the expression, activity, function or phenotype arising therefrom.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, ²N-methylguanosine and ^{2,2}N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “bond strength” or “base pair strength” refers to the strength of the interaction between pairs of nucleotides (or nucleotide analogs) on opposing strands of an oligonucleotide duplex, due primarily to H-bonding, van der Waals interactions, and the like between said nucleotides (or nucleotide analogs).

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide. In yet another embodiment, the destabilizing nucleotide is capable of forming a bulge, wherein the destabilizing nucleotide does not pair with the second nucleotide.

As used herein, the term “base pair” refers to the interaction between pairs of nucleotides (or nucleotide analogs) on opposing strands of an oligonucleotide duplex, due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g. certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portions of universal nucleotides typically comprise a nitrogen-containing aromatic heterocyclic moiety.

As used herein, the terms “sufficient complementarity” or “sufficient degree of complementarity” mean that an oligonucleotide sequence is sufficiently complementary to bind a desired target oligonucleotide.

Various methodologies of the instant invention include step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a methodology, as described herein. For example, a transcription rate, mRNA level, translation rate, protein level, biological activity, cellular characteristic or property, genotype, phenotype, etc. can be determined prior to introducing a synthetic RNA or DNA agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Synthetic RNA or DNA agents of the invention may be directly introduced into the cell (i.e., intracellularly), or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where synthetic RNA or DNA agent may be introduced.

The synthetic RNA or DNA agents of the invention can be introduced using nucleic acid delivery methods known in art including injection of a solution containing the nucleic acid, bombardment by particles covered by the RNA agent, soaking the cell or organism in a solution of the RNA agent, or electroporation of cell membranes in the presence of the RNA agent. Other methods known in the art for introducing nucleic acids to cells may be used, such as lipid-mediated carrier transport, chemical-mediated transport, and cationic liposome transfection such as calcium phosphate, and the like. The synthetic RNA or DNA agent may be introduced along with other components that perform one or more of the following activities: enhance nucleic acid uptake by the cell or otherwise increase inhibition of the target gene.

Physical methods of introducing nucleic acids include injection of a solution containing the biosensor, bombardment by particles covered by the biosensor, soaking the cell or organism in a solution of the biosensor, or electroporation of cell membranes in the presence of the biosensor. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, inhibit annealing of single strands, stabilize the single strands, or other-wise increase inhibition of the target gene.

The synthetic RNA or DNA agent may be directly introduced into the cell (i.e., intracellularly), or introduced extracellularly into a cavity, interstitial space, into the circulation of an organism, introduced orally, or may be introduced by bathing a cell or organism in a solution containing the RNA or DNA. Vascular or extravascular circulation, the blood or lymph system, and the cerebrospinal fluid are sites where the RNA or DNA may be introduced.

A target cell may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

The synthetic RNA or DNA agent may be introduced in an amount which allows delivery of at least one copy per cell. Higher doses (e.g., at least 5, 10, 100, 500 or 1000 copies per cell) of material may yield more effective inhibition; lower doses may also be useful for specific applications.

In an exemplary aspect, the efficacy of a biosensor of the invention is tested for its ability to specifically modulate transcription, translation, alternative splicing and/or mRNA stability of a target in a cell. Cells can be transfected with one or more biosensors described herein. Selective reduction in target DNA, target RNA (e.g., mRNA) and/or target protein is measured. Reduction of target DNA, RNA or protein can be compared to levels of target DNA, RNA or protein in the absence of a biosensor or in the presence of a biosensor that does not target the DNA, RNA or protein. Exogenously-introduced DNA, RNA or protein can be assayed for comparison purposes. When utilizing neuronal cells, which are known to be somewhat resistant to standard transfection techniques, it may be desirable to introduce biosensors by passive uptake.

“Treatment,” or “treating,” as used herein, is defined as the application or administration of a therapeutic agent (e.g., a synthetic RNA or DNA agent) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder, by administering to the subject a therapeutic agent (e.g., a synthetic RNA or DNA agent or vector or transgene encoding same). Subjects at risk for the disease can be identified by, for example, any or a combination of diagnostic or prognostic. Administration of a prophylactic agent can occur prior to the manifestation of symptoms characteristic of the disease or disorder, such that the disease or disorder is prevented or, alternatively, delayed in its progression.

Another aspect of the invention pertains to methods treating subjects therapeutically, i.e., alter onset of symptoms of a disease or disorder. In an exemplary embodiment, the modulatory method of the invention involves contacting a cell expressing disorder with a therapeutic agent (e.g., a synthetic RNA or DNA agent or vector or transgene encoding same) that is specific for one or more target sequences, such that a sequence specific interaction with the target sequence is achieved. These methods can be performed in vitro (e.g., by culturing the cell with the agent) or, alternatively, in vivo (e.g., by administering the agent to a subject).

With regards to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype,” or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic agents can be tested in an appropriate animal model. For example, a synthetic RNA or DNA agent (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

A pharmaceutical composition containing a synthetic RNA or DNA agent of the invention can be administered to any patient diagnosed as having or at risk for developing a disorder. In one embodiment, the patient is diagnosed as having a disorder, and the patient is otherwise in general good health. For example, the patient is not terminally ill, and the patient is likely to live at least 2, 3, 5 or more years following diagnosis. The patient can be treated immediately following diagnosis, or treatment can be delayed until the patient is experiencing more debilitating symptoms. In another embodiment, the patient has not reached an advanced stage of the disease.

An a synthetic RNA or DNA agent can be administered at a unit dose less than about 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of a synthetic RNA or DNA agent per kg of bodyweight. The unit dose, for example, can be administered by injection (e.g., intravenous or intramuscular, intrathecally, or directly into the brain), an inhaled dose, or a topical application. Particularly preferred dosages are less than 2, 1, or 0.1 mg/kg of body weight.

Delivery of a synthetic RNA or DNA agent directly to an organ can be at a dosage on the order of about 0.00001 mg to about 3 mg per organ, or preferably about 0.0001-0.001 mg per organ, about 0.03-3.0 mg per organ, about 0.1-3.0 mg per eye or about 0.3-3.0 mg per organ. The dosage can be an amount effective to treat or prevent a disorder. In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In one embodiment, a subject is administered an initial dose, and one or more maintenance doses of a synthetic RNA or DNA agent. The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days.

Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable. In one embodiment, a pharmaceutical composition includes a plurality of a synthetic RNA or DNA agent species. In another embodiment, the a synthetic RNA or DNA agent species has sequences that are non-overlapping and non-adjacent to another species with respect to a naturally occurring target sequence. In another embodiment, the plurality of a synthetic RNA or DNA agent species is specific for different naturally occurring targets. In another embodiment, the plurality of a synthetic RNA or DNA agent species target two or more target sequences (e.g., two, three, four, five, six, or more target sequences).

Following successful treatment, it may be desirable to have the patient undergo maintenance therapy to prevent the recurrence of the disease state, wherein the compound of the invention is administered in maintenance doses, ranging from 0.01 μg to 100 g per kg of body weight (see U.S. Pat. No. 6,107,094).

The concentration of the a synthetic RNA or DNA agent composition is an amount sufficient to be effective in treating or preventing a disorder or to regulate a physiological condition in humans. The concentration or amount of a synthetic RNA or DNA agent administered will depend on the parameters determined for the agent and the method of administration, e.g. nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an a synthetic RNA or DNA agent can include a single treatment or, preferably, can include a series of treatments. It will also be appreciated that the effective dosage of a synthetic RNA or DNA agent for treatment may increase or decrease over the course of a particular treatment. Changes in dosage may result and become apparent from the results of diagnostic assays as described herein. For example, the subject can be monitored after administering a synthetic RNA or DNA agent composition. Based on information from the monitoring, an additional amount of the synthetic RNA or DNA agent composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models.

The invention pertains to uses of the above-described agents for prophylactic and/or therapeutic treatments as described Infra. Accordingly, the modulators (e.g., synthetic RNA or DNA agents) of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous (IV), intradermal, subcutaneous (SC or SQ), intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The synthetic RNA or DNA agent can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

The synthetic RNA or DNA agent can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a container, pack or dispenser together with optional instructions for administration.

As defined herein, a therapeutically effective amount of synthetic RNA or DNA agent (i.e., an effective dosage) depends on the synthetic RNA or DNA agent selected. For instance, single dose amounts in the range of approximately 1 μg to 1000 mg may be administered; in some embodiments, 10, 30, 100 or 1000 μg may be administered. In some embodiments, 1-5 g of the compositions can be administered. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a synthetic RNA or DNA agent can include a single treatment or, preferably, can include a series of treatments.

The nucleic acid molecules of the invention can be inserted into expression constructs, e.g., viral vectors, retroviral vectors, expression cassettes, or plasmid viral vectors, e.g., using methods known in the art, including but not limited to those described in Xia et al., (2002), Supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The route of delivery can be dependent on the disorder of the patient. In certain exemplary embodiments, a subject can be administered a synthetic RNA or DNA agent of the invention by IV or SC administration. In addition to a synthetic RNA or DNA agent of the invention, a patient can be administered a second therapy, e.g., a palliative therapy and/or disease-specific therapy. The secondary therapy can be, for example, symptomatic (e.g., for alleviating symptoms), protective (e.g., for slowing or halting disease progression), or restorative (e.g., for reversing the disease process).

In general, a synthetic RNA or DNA agent of the invention can be administered by any suitable method. As used herein, topical delivery can refer to the direct application of a synthetic RNA or DNA agent to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the synthetic RNA or DNA agent to the epidermis or dermis of a subject, or to specific strata thereof, or to an underlying tissue.

Compositions for intrathecal or intraventricular administration may include sterile aqueous solutions which may also contain buffers, diluents and other suitable additives.

Compositions for intrathecal or intraventricular administration preferably do not include a transfection reagent or an additional lipophilic moiety besides, for example, the lipophilic moiety attached to the synthetic RNA or DNA agent.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

A synthetic RNA or DNA agent of the invention can be administered to a subject by pulmonary delivery. Pulmonary delivery compositions can be delivered by inhalation of a dispersion so that the composition within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver drugs that may be readily formulated as dry powders. A synthetic RNA or DNA agent composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The types of pharmaceutical excipients that are useful as carriers include stabilizers such as Human Serum Albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-β-cyclodextrin; and polysaccharides, such as raffinose, maltodextrins, dextrans, and the like; alditols, such as mannitol, xylitol, and the like. A preferred group of carbohydrates includes lactose, trehalose, raffinose maltodextrins, and mannitol. Suitable polypeptides include aspartame. Amino acids include alanine and glycine, with glycine being preferred.

Suitable pH adjusters or buffers include organic salts prepared from organic acids and bases, such as sodium citrate, sodium ascorbate, and the like; sodium citrate is preferred.

A synthetic RNA or DNA agent of the invention can be administered by oral and nasal delivery. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily. In one embodiment, a synthetic RNA or DNA agent administered by oral or nasal delivery has been modified to be capable of traversing the blood-brain barrier.

In one embodiment, unit doses or measured doses of a composition that include synthetic RNA or DNA agents are dispensed by an implanted device. The device can include a sensor that monitors a parameter within a subject. For example, the device can include a pump, such as an osmotic pump and, optionally, associated electronics.

A synthetic RNA or DNA agent can be packaged in a viral natural capsid or in a chemically or enzymatically produced artificial capsid or structure derived therefrom.

In certain other aspects, the invention provides kits that include a suitable container containing a pharmaceutical formulation of a synthetic RNA or DNA agent. In certain embodiments the individual components of the pharmaceutical formulation may be provided in one container. Alternatively, it may be desirable to provide the components of the pharmaceutical formulation separately in two or more containers, e.g., one container for a synthetic RNA or DNA agent preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1: Scaffolding Selection Libraries With Information From Biological RNAs

Examination of small biological RNAs with multi-helix packing (i.e., tertiary folding) indicated two recurrent architectures that may be considered privileged scaffolds. The first is the H-type pseudoknot, which is broadly found in biological RNAs including small ribozyme ribosomal frame-shifting elements in viral mRNAs, and natural and synthetic aptamers. However, from a design perspective this fold can be difficult to engineer. The other is the three-way junction (3WJ) supported by a remote tertiary interaction that organizes the helical arrangement around the junction. This fold is more suitable for the design of RNA devices that incorporate aptamers as it positions a designable helical element (called the P1 helix) proximal to the ligand-binding site typically housed in the junction.

Within the three-way junction fold group, there are a large selection of potential candidates that could be used to scaffold an initial library of sequences for in vitro selection. Three are exemplified herein: the aptamer domain of the B. subtilis xpt-pbuX guanine riboswitch (referred to herein as “GR”), the aptamer domain of the Vibrio cholerae Vc2 cyclic di-GMP riboswitch (referred to herein as “CDG”), and the Schistosoma mansoni hammerhead ribozyme (referred to herein as “HH”) (FIG. 1). In each of these parental RNA scaffolds, the junction hosts the key biological activity proximal to the P1 helix that can serve as the secondary structural bridge to a readout domain.

Starting libraries were designed to preserve the overall secondary and tertiary structure of the scaffold while randomizing a sufficient number of nucleotides in the junction to ensure adequate pool diversity such that winners emerge. All nucleotides in the joining strands of the junction were randomized (equal populations of the four nucleotides at each position), as well as at least one base pair in each helix proximal to the junction (FIG. 1). For the GR scaffold, this yielded an initial library of 23 randomized nucleotide positions equating to a library size of about 7×10¹³ sequences (4²³ sequences); the CDG and HH contain similar levels of diversity (21 randomized nucleotide positions equating to a library size of about 4×10¹² sequences; 4²¹ sequences). This number of sequences is theoretically fully represented in the initial pool of RNA with at least five-fold redundancy. While this is substantially lower diversity than what is recommended for a typical selection, novel aptamers have been attained from starting pools with even more limited sampling of sequence space.

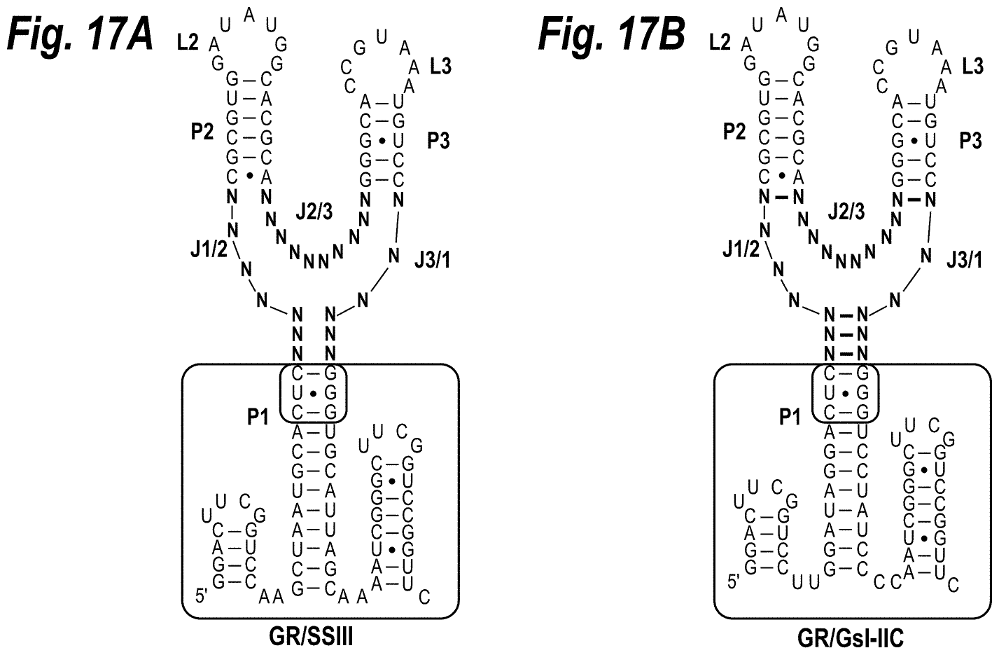

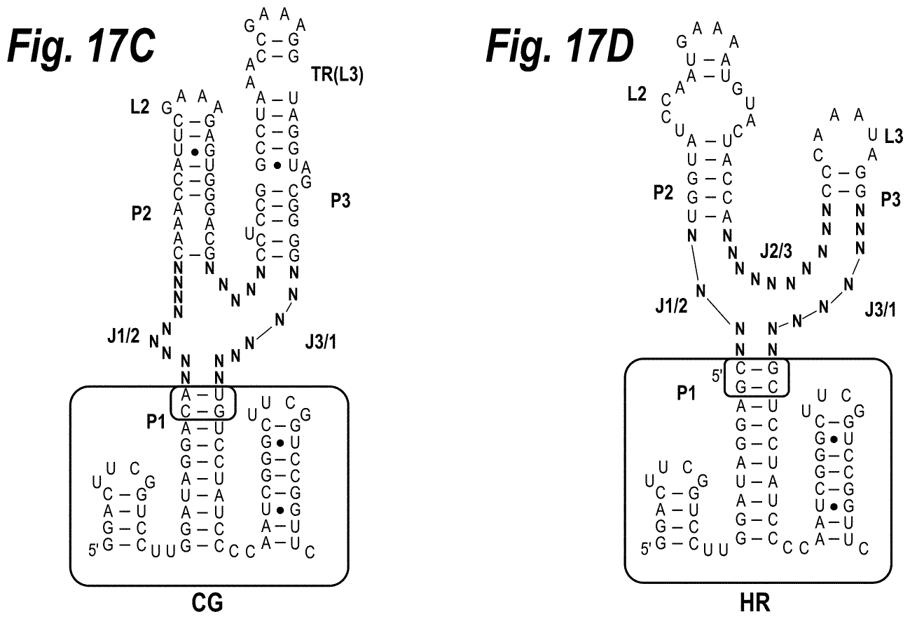

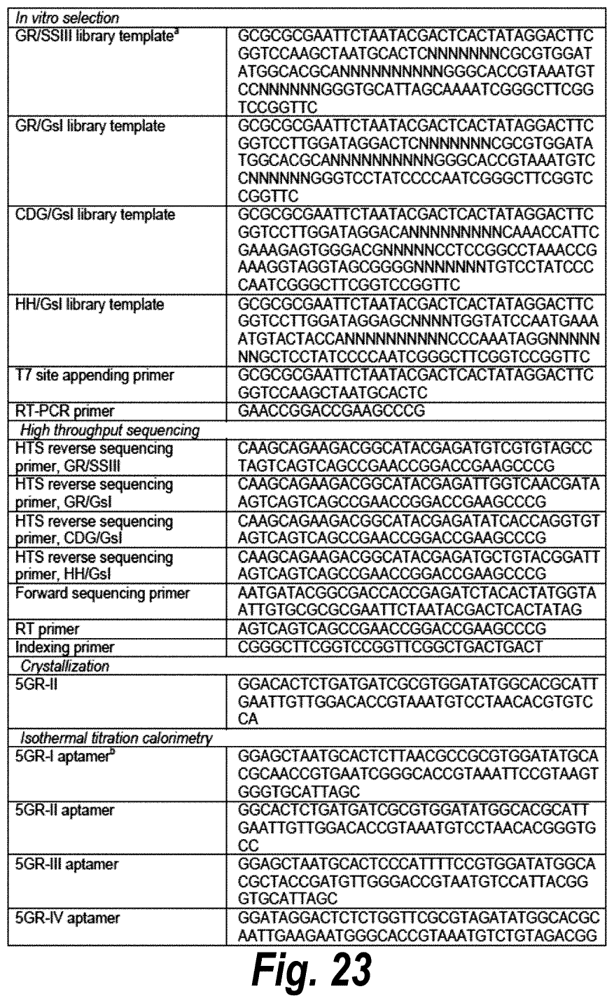

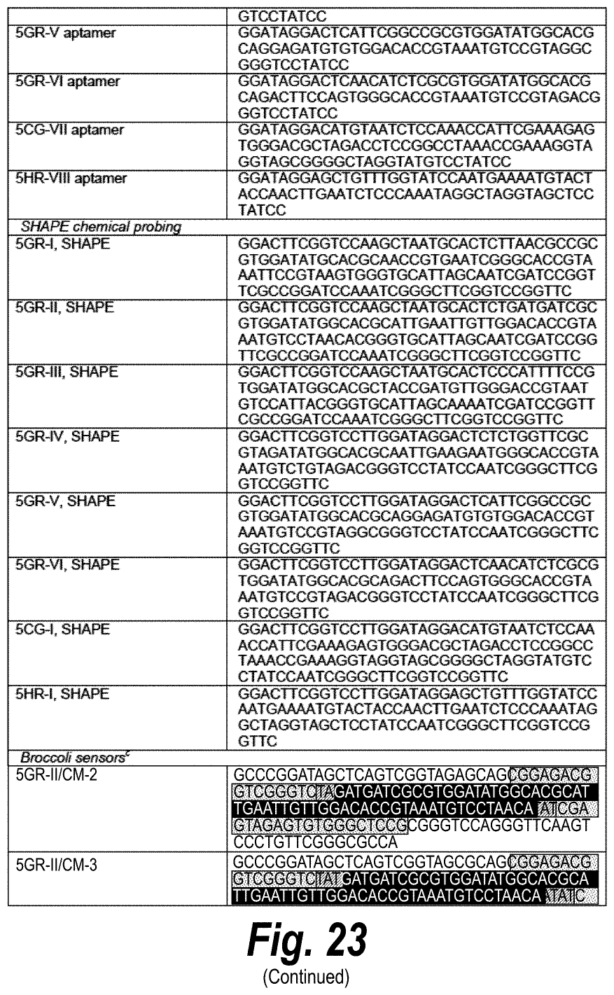

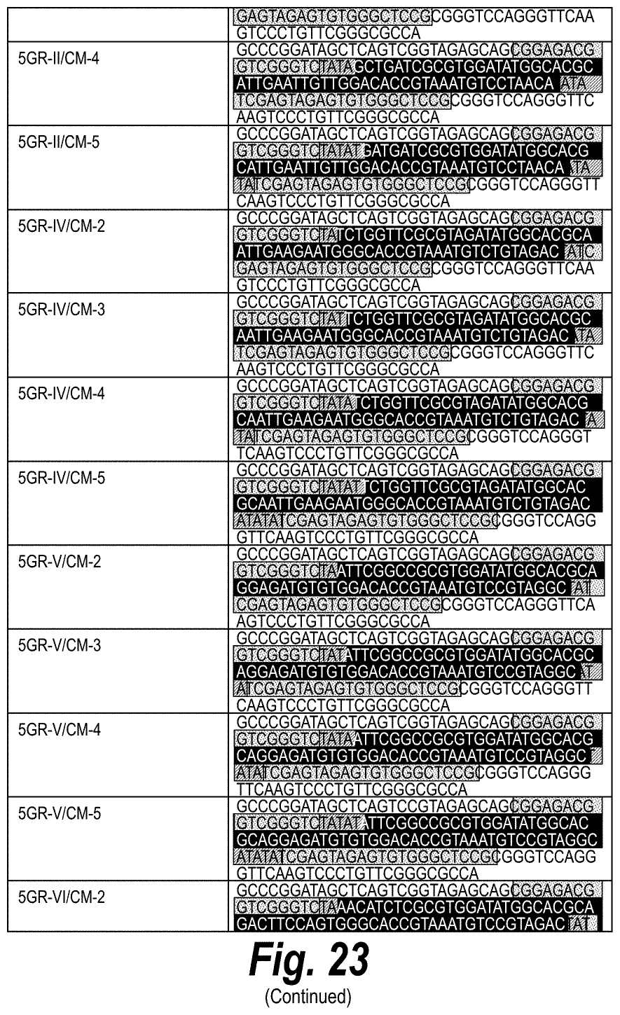

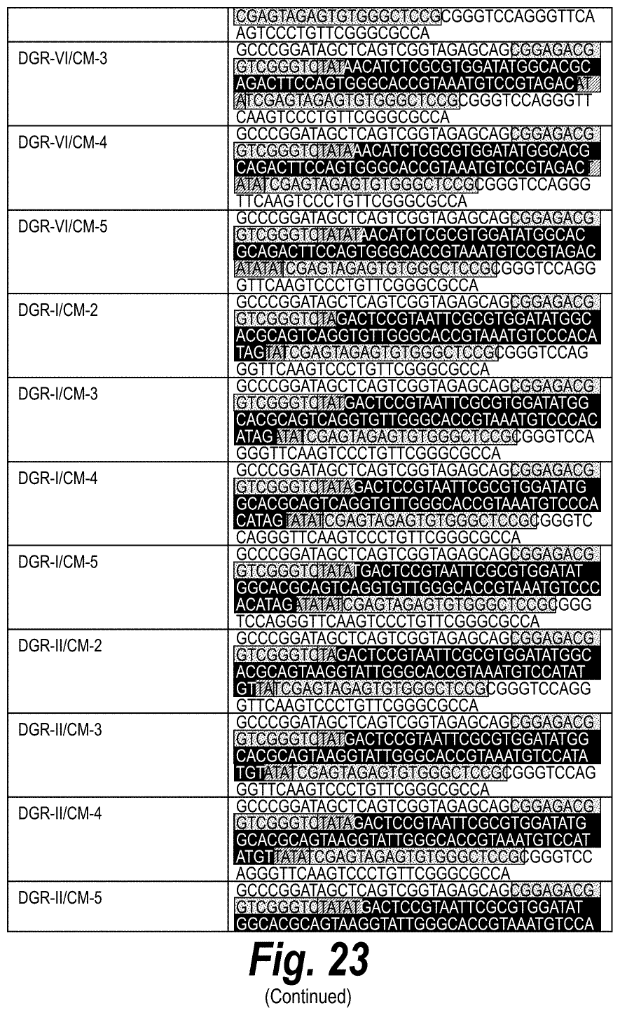

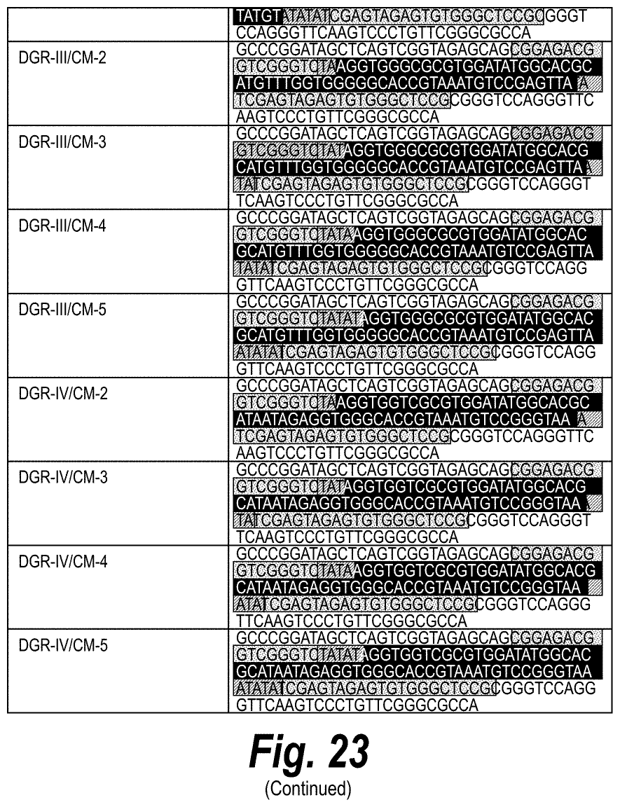

The three scaffolds were integrated into a library cassette with specific design features. The P1 helix of each scaffold containing the initial and terminal bases of the scaffold sequences was replaced in all libraries with a designed helix-containing structured amplification cassettes based upon those developed for Selective 2′-Hydroxyl Acylation analyzed by Primer Extension (“SHAPE”) chemical probing of RNA structure (FIG. 17). This ensures that the constant regions necessary for replication are structured and less likely to be incorporated into the selected aptamer. To further minimize the potential for the constant regions to participate in formation of the ligand-binding site, the P1 helix was extended to at least ten base pairs. Full sequences of DNA templates encoding the initial starting libraries are given in Table 1 and FIG. 23.

TABLE 1
Sequences of oligonucleotides and templates.

| | |
|---|---|
| Oligonucleotide | Sequence |

In vitro selection

| | |
|---|---|
| GR/SSIII library template^{a} | GCGCGCGAATTCTAATACGACTCACTATAGGACT |
| | TCGGTCCAAGCTAATGCACTCNNNNNNNCGCGT |
| | GGATATGGCACGCANNNNNNNNNGGGCACCGT |
| | AAATGTCCNNNNNNGGGTGCATTAGCAAAATCG |
| | GGCTTCGGTCCGGTTC |
| GR/GsI library template | GCGCGCGAATTCTAATACGACTCACTATAGGACT |
| | TCGGTCCTTGGATAGGACTCNNNNNNNCGCGTG |
| | GATATGGCACGCANNNNNNNNNGGGCACCGTA |
| | AATGTCCNNNNNNGGGTCCTATCCCCAATCGGG |
| | CTTCGGTCCGGTTC |
| CDG/GsI library template | GCGCGCGAATTCTAATACGACTCACTATAGGACT |
| | TCGGTCCTTGGATAGGACANNNNNNNNCAAAC |
| | CATTCGAAAGAGTGGGACGNNNNNCCTCCGGCC |
| | TAAACCGAAAGGTAGGTAGCGGGGNNNNNNNT |
| | GTCCTATCCCCAATCGGGCTTCGGTCCGGTTC |
| HH/GsI library template | GCGCGCGAATTCTAATACGACTCACTATAGGACT |
| | TCGGTCCTTGGATAGGAGCNNNNTGGTATCCAA |
| | TGAAAATGTACTACCANNNNNNNNNNCCCAAAT |
| | AGGNNNNNNNGCTCCTATCCCCAATCGGGCTTC |
| | GGTCCGGTTC |
| T7 site appending primer | GCGCGCGAATTCTAATACGACTCACTATAGGAC |
| | TTCGGTCCAAGCTAATGCACTC |
| RT-PCR primer | GAACCGGACCGAAGCCCG |

High throughput sequencing

| | |
|---|---|
| HTS reverse sequencing primer, | CAAGCAGAAGACGGCATACGAGATGTCGTGTAG |
| GR/SSIII | CCTAGTCAGTCAGCCGAACCGGACCGAAGCCCG |
| HTS reverse sequencing primer, | CAAGCAGAAGACGGCATACGAGATTGGTCAACG |
| GR/GsI | ATAAGTCAGTCAGCCGAACCGGACCGAAGCCCG |
| HTS reverse sequencing primer, | CAAGCAGAAGACGGCATACGAGATATCACCAGG |
| CDG/GsI | TGTAGTCAGTCAGCCGAACCGGACCGAAGCCCG |
| HTS reverse sequencing primer, | CAAGCAGAAGACGGCATACGAGATGCTGTACGG |
| HH/GsI | ATTAGTCAGTCAGCCGAACCGGACCGAAGCCCG |
| Forward sequencing primer | AATGATACGGCGACCACCGAGATCTACACTATG |
| | GTAATTGTGCGCGCGAATTCTAATACGACTCACT |
| | ATAG |
| RT primer | AGTCAGTCAGCCGAACCGGACCGAAGCCCG |
| Indexing primer | CGGGCTTCGGTCCGGTTCGGCTGACTGACT |

Crystallization

| | |
|---|---|
| 5HTP-II | GGACACTCTGATGATCGCGTGGATATGGCACGC |
| | ATTGAATTGTTGGACACCGTAAATGTCCTAACAC |
| | GTGTCCA |

Isothermal titration calorimetry

| | |
|---|---|
| 5HTP-I aptamer^{b} | GGAGCTAATGCACTCTTAACGCCGCGTGGATAT |
| | GCACGCAACCGTGAATCGGGCACCGTAAATTCC |
| | GTAAGTGGGTGCATTAGC |
| 5HTP-II aptamer | GGCACTCTGATGATCGCGTGGATATGGCACGCA |
| | TTGAATTGTTGGACACCGTAAATGTCCTAACACG |
| | GGTGCC |
| 5HTP-III aptamer | GGAGCTAATGCACTCCCATTTTCCGTGGATATGG |
| | CACGCTACCGATGTTGGGACCGTAATGTCCATTA |
| | CGGGTGCATTAGC |
| 5HTP-IV aptamer | GGATAGGACTCTCTGGTTCGCGTAGATATGGCAC |
| | GCAATTGAAGAATGGGCACCGTAAATGTCTGTA |
| | GACGGGTCCTATCC |
| 5HTP-V aptamer | GGATAGGACTCATTCGGCCGCGTGGATATGGCA |
| | CGCAGGAGATGTGTGGACACCGTAAATGTCCGT |
| | AGGCGGGTCCTATCC |
| 5HTP-VI aptamer | GGATAGGACTCAACATCTCGCGTGGATATGGCA |
| | CGCAGACTTCCAGTGGGCACCGTAAATGTCCGT |
| | AGACGGGTCCTATCC |
| 5HTP-VII aptamer | GGATAGGACATGTAATCTCCAAACCATTCGAAA |
| | GAGTGGGACGCTAGACCTCCGGCCTAAACCGAA |
| | AGGTAGGTAGCGGGGCTAGGTATGTCCTATCC |
| 5HTP-VIII aptamer | GGATAGGAGCTGTTTGGTATCCAATGAAAATGT |
| | ACTACCAACTTGAATCTCCCAAATAGGCTAGGTA |
| | GCTCCTATCC |

SHAPE chemical probing

| | |
|---|---|
| 5HTP-I, SHAPE | GGACTTCGGTCCAAGCTAATGCACTCTTAACGCC |
| | GCGTGGATATGCACGCAACCGTGAATCGGGCAC |
| | CGTAAATTCCGTAAGTGGGTGCATTAGCAATCG |
| | ATCCGGTTCGCCGGATCCAAATCGGGCTTCGGTC |
| | CGGTTC |
| 5HTP-II, SHAPE | GGACTTCGGTCCAAGCTAATGCACTCTGATGATC |
| | GCGTGGATATGGCACGCATTGAATTGTTGGACA |
| | CCGTAAATGTCCTAACACGGGTGCATTAGCAAT |
| | CGATCCGGTTCGCCGGATCCAAATCGGGCTTCG |
| | GTCCGGTTC |
| 5HTP-III, SHAPE | GGACTTCGGTCCAAGCTAATGCACTCCCATTTTC |
| | CGTGGATATGGCACGCTACCGATGTTGGGACCG |
| | TAATGTCCATTACGGGTGCATTAGCAAAATCGA |
| | TCCGGTTCGCCGGATCCAAATCGGGCTTCGGTCC |
| | GGTTC |
| 5HTP-IV, SHAPE | GGACTTCGGTCCTTGGATAGGACTCTCTGGTTCG |
| | CGTAGATATGGCACGCAATTGAAGAATGGGCAC |
| | CGTAAATGTCTGTAGACGGGTCCTATCCAATCG |
| | GGCTTCGGTCCGGTTC |
| 5HTP-V, SHAPE | GGACTTCGGTCCTTGGATAGGACTCATTCGGCCG |
| | CGTGGATATGGCACGCAGGAGATGTGTGGACAC |
| | CGTAAATGTCCGTAGGCGGGTCCTATCCAATCG |
| | GGCTTCGGTCCGGTTC |
| 5HTP-VI, SHAPE | GGACTTCGGTCCTTGGATAGGACTCAACATCTCG |
| | CGTGGATATGGCACGCAGACTTCCAGTGGGCAC |
| | CGTAAATGTCCGTAGACGGGTCCTATCCAATCG |
| | GGCTTCGGTCCGGTTC |
| 5HTP-VII, SHAPE | GGACTTCGGTCCTTGGATAGGACATGTAATCTCC |
| | AAACCATTCGAAAGAGTGGGACGCTAGACCTCC |
| | GGCCTAAACCGAAAGGTAGGTAGCGGGGCTAGG |
| | TATGTCCTATCCAATCGGGCTTCGGTCCGGTTC |
| 5HTP-VIII, SHAPE | GGACTTCGGTCCTTGGATAGGAGCTGTTTGGTAT |
| | CCAATGAAAATGTACTACCAACTTGAATCTCCC |
| | AAATAGGCTAGGTAGCTCCTATCCAATCGGGCT |
| | TCGGTCCGGTTC |

Broccoli sensors

| | |
|---|---|
| 5HTP-II/A-Broccoli | GGACGGAGACGGTCGGGTCATATGATGATCGCG |
| | TGGATATGGCACGCATTGAATTGTTGGACACCG |
| | TAAATGTCCTAACAATATTCGAGTAGAGTGTGG |
| | GCTCCGTCC |
| 5HTP-II/U-Broccoli | GGACGGAGACGGTCGGGTCTATAGATGATCGCG |
| | TGGATATGGCACGCATTGAATTGTTGGACACCG |
| | TAAATGTCCTAACATATATCGAGTAGAGTGTGG |
| | GCTCCGTCC |
| 5HTP-IV/A-Broccoli | GGACGGAGACGGTCGGGTCATATTCTGGTTCGC |
| | GTAGATATGGCACGCAATTGAAGAATGGGCACC |
| | GTAAATGTCTGTAGACATATTCGAGTAGAGTGT |
| | GGGCTCCGTCC |
| 5HTP-IV/U-Broccoli | GGACGGAGACGGTCGGGTCTATATCTGGTTCGC |
| | GTAGATATGGCACGCAATTGAAGAATGGGCACC |
| | GTAAATGTCTGTAGACTATATCGAGTAGAGTGT |
| | GGGCTCCGTCC |
| 5HTP-VII/A-Broccoli | GGACGGAGACGGTCGGGTCATATATATTCGATG |
| | TAATCTCCAAACCATTCGAAAGAGTGGGACGCT |
| | AGACCTCCGGCCTAAACCGAAAGGTAGGTAGCG |
| | GGGCTAGGTAGTAGAGTGTGGGCTCCGTCC |
| 5HTP-VII/U-Broccoli | GGACGGAGACGGTCGGGTCTATATGTAATCTCC |
| | AAACCATTCGAAAGAGTGGGACGCTAGACCTCC |
| | GGCCTAAACCGAAAGGTAGGTAGCGGGGCTAGG |
| | TATATATCGAGTAGAGTGTGGGCTCCGTCC |
| 5HTP-VIII/A-Broccoli | GGACGGAGACGGTCGGGTCATATTGTTTGGTAT |
| | CCAATGAAAATGTACTACCAACTTGAATCTCCC |
| | AAATAGGCTAGGTAATATTCGAGTAGAGTGTGG |
| | GCTCCGTCC |
| 5HTP-VIII/U-Broccoli | GGACGGAGACGGTCGGGTCTATATGTTTGGTAT |
| | CCAATGAAAATGTACTACCAACTTGAATCTCCC |
| | AAATAGGCTAGGTATATATCGAGTAGAGTGTGG |
| | GCTCCGTCC |

In vitro single turnover transcription

| | |
|---|---|
| 5HTP-IV/pbuE riboswitch | AATATTGAGCTGTTGACAATTAATCATCGGCTCG |
| | TATAATGTGTGGAATTAAATAGCTATTATCAGGA |
| | TTTTTCTGGTTCGCGTAGATATGGCACGCAATTG |
| | AAGAATGGGCACCGTAAATGTCTGTAGACAAAA |
| | TCCTGATTACAAAATTTGTTTATGACATTTTTTGT |
| | AATCAGGATTTTTTTATTTATCAAAACATTTAAG |
| | TAAAGGAGTTTGTTATG |

^{a“}N” represents a position where the composition of A, C, G and T is approximately 25% each.
^{b}RNA aptamer and sensor sequences are given as their equivalent DNA sequences.

A further complicating issue for scaffold selection was the low fidelity of viral reverse transcriptases (RTs). Engineering of MMLV RT to improve thermostability and processivity to create the most commonly used versions of this enzyme decreased its already low fidelity. Misincorporation or deletion of nucleotides in conserved sequences of the scaffold readily disrupts tertiary interactions that stabilize the global fold. RNAs lacking structure are amplified more efficiently by RT, which can introduce a significant bias during the replication step of each round of selection, which in part likely leads to the “tyranny of small motifs” phenomenon observed in selection. To address this, a recently characterized RT derived from a mobile group II intron from the thermophile Geobacillus stearothermophilus (GsI-IIC-MRF or “GsI”) that retains activity up to 70° C. (versus 55° C. for SSIII) and has inherently higher fidelity than MMLV-derived RTs was adopted. For comparison, the GR scaffold selection was performed with an RT derived from MMLV (SuperScript III or “SSIII”) along with GsI.

### Example 2: Scaffolded Selection Against 5HTP Yields Many Potential Aptamers

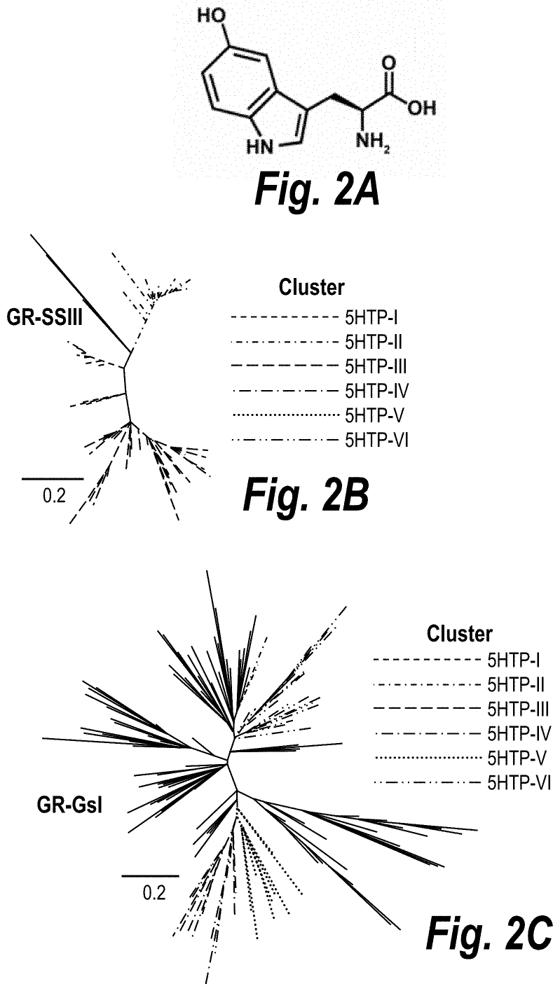

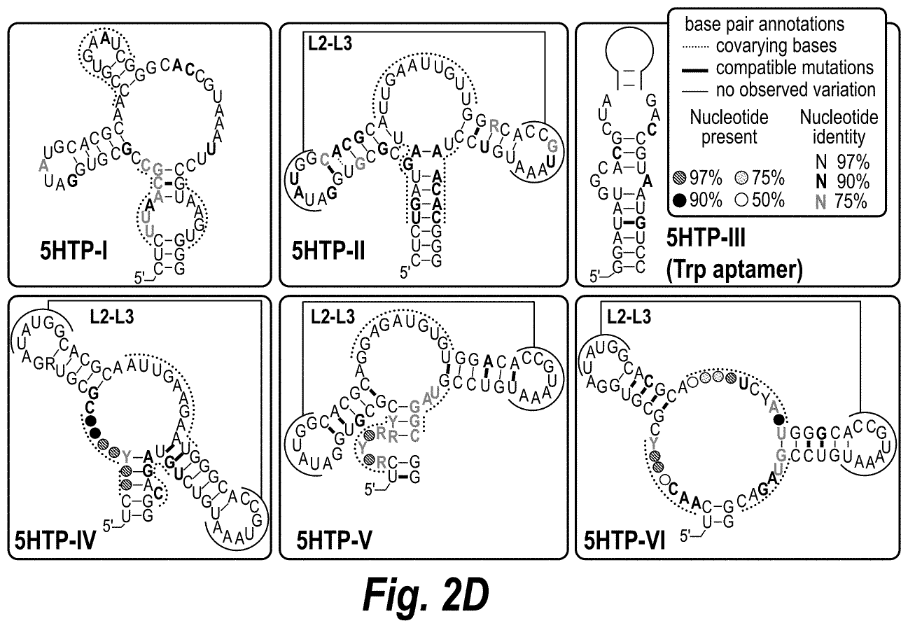

The target for selection was 5-hydroxy-L-tryptophan (5HTP; FIG. 2A), the immediate biosynthetic precursor of serotonin, which was immobilized on a solid matrix via its carboxylate group. Seven rounds of selection with each library were carried out, with counterselections against L-tryptophan and increasingly stringent washing procedures in later rounds. In the SSIII selection, a conventional SELEX protocol was adopted in which the affinity column was extensively washed in early rounds prior to competitive elution to remove nonbinding RNAs. Competitive elution was initially observed in round four and peaked at >50% of total input RNA in round six. The GsI selections used a less stringent protocol than generally recommended in which roughly the final 10% of total RNA left on the column under competitive elution was collected for amplification in the initial four rounds to preserve sequence diversity in the pool before increasing wash stringency. Details of the selections are given in Examples 6-8 and Table 2.

TABLE 2
Selection conditions per cycle.

| Round | [RNA] pmol | Washes* | Notes |
|---|---|---|---|
| SS-III selection | | | |
| 1 | 1000 | 3 | Counter selection against AcO-sepharose |
| 2 | 400 | 6 | Counter selection against AcO-sepharose |
| 3 | 400 | 10 | |
| 4 | 400 | 10 | |
| 5 | 100 | 10 | |
| 6 | 100 | 10 | 30 second counter selection with 100 mM L-tryptophan |
| 7 | 100 | 10 | 30 second counter selection with 100 mM L-tryptophan |
| GsI selections | | | |
| 1 | 1000 | 3 | Counter selection against AcO-sepharose |
| 2 | 400 | 3 | Counter selection against AcO-sepharose |
| 3 | 400 | 5 | |
| 4 | 400 | 5 | |
| 5 | 400 | 10 | |
| 6 | 200 | 6 | 30 second counter selection with 100 mM L-tryptophan |
| 7 | 200 | 10 | 30 second counter selection with 100 mM L-tryptophan |

*Each wash constituted 3 column volumes of buffer

In preserving sequence diversity and minimizing early stochastic events, a combination of next generation sequencing (NGS) and downstream bioinformatic analysis was relied on to reveal potential aptamers and elucidate key features of the selection. For each selection, >200,000 reads were obtained for RNA from the final round, and resultant sequences were clustered and maximum-likelihood trees generated. Comparison of the SSIII and GsI selections using the GR scaffold revealed several important features.

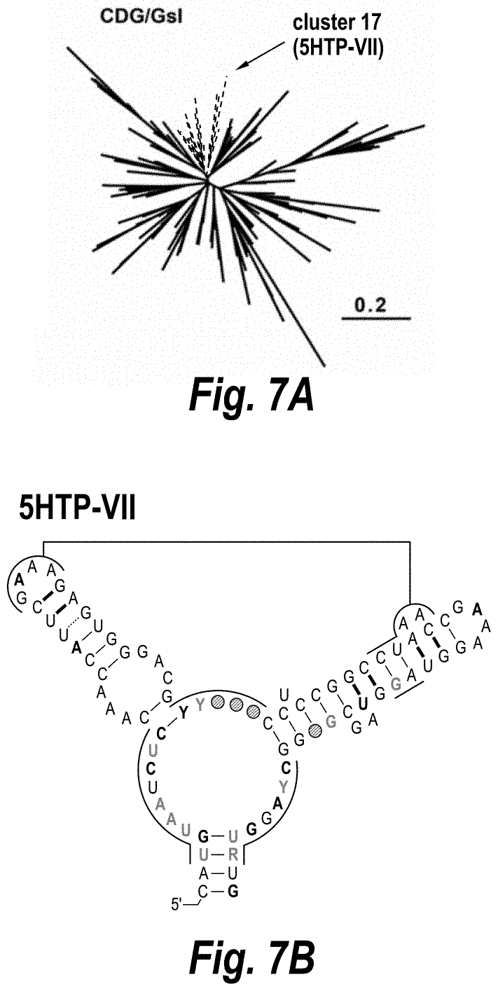

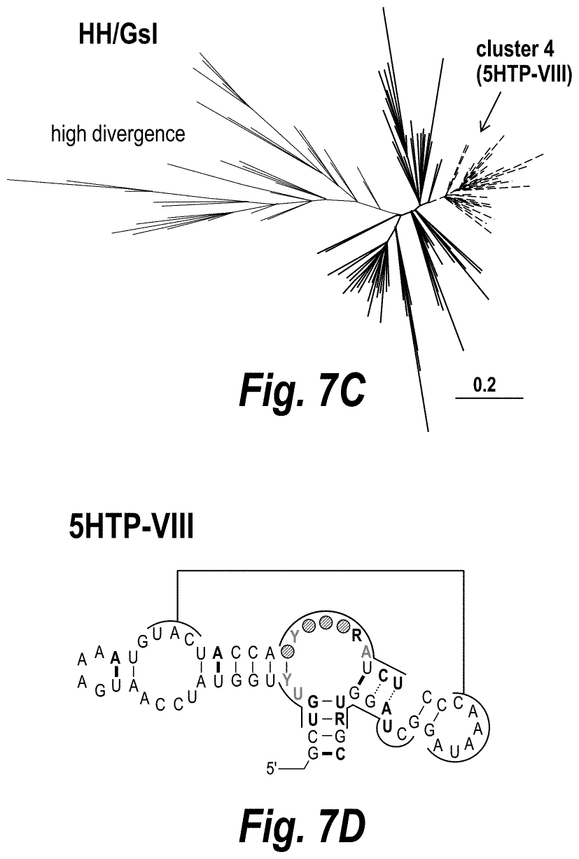

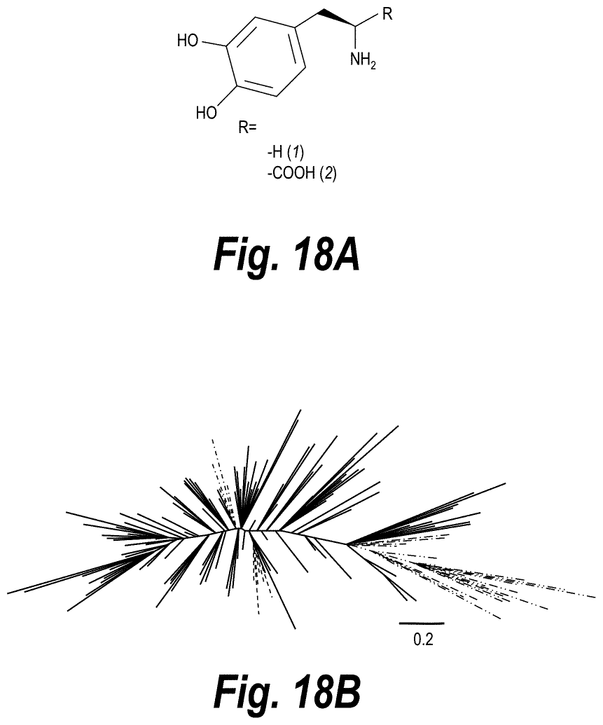

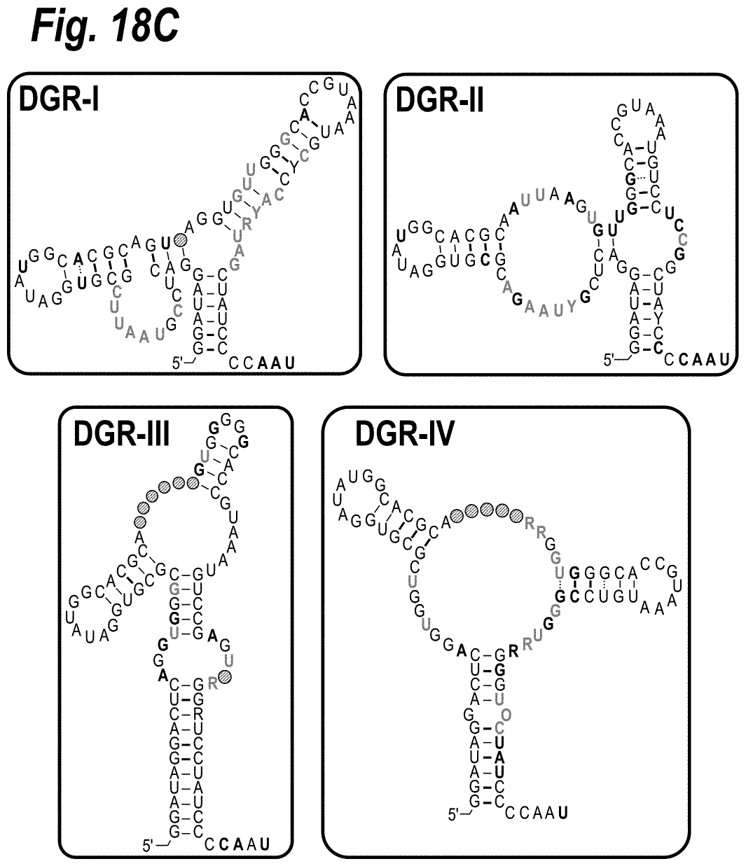

A distance matrix of the GR/SSIII selection clearly showed only a few isolated clusters, and within each cluster the sequences have a high degree of internal relatedness (FIG. 2B). The majority (>80%) of sequences cluster into three distinct sequence-related families, referred to as 5HTP-I, -II, and -III (FIG. 2D), with the remaining clustering into small populations that are difficult to interpret. This is typical of a traditional SELEX where single isolates are often identified and further mutagenesis and selection are necessary to obtain covariation information. In contrast, the GR/GsI selection yielded more diverse clusters with higher sampling of sequences that populate regions between major clusters (FIGS. 2C and 2D). The CDG and HH selections with GsI are similarly diverse in their sequence space with many potential aptamers (FIG. 7). While traditional selection approaches often rely upon over-selection to facilitate finding an aptamer with limited sequence information, preservation of the diversity of winners and sequence analysis by NGS allowed for a more thorough analysis of conservation and covariation patterns, aiding in determining consensus aptamer sequences. Similar results were observed in the GR-scaffolded L-DOPA selection (FIG. 18). A subset of 250 sequences from each of the clusters that yielded validated 5HTP aptamers is described further herein.

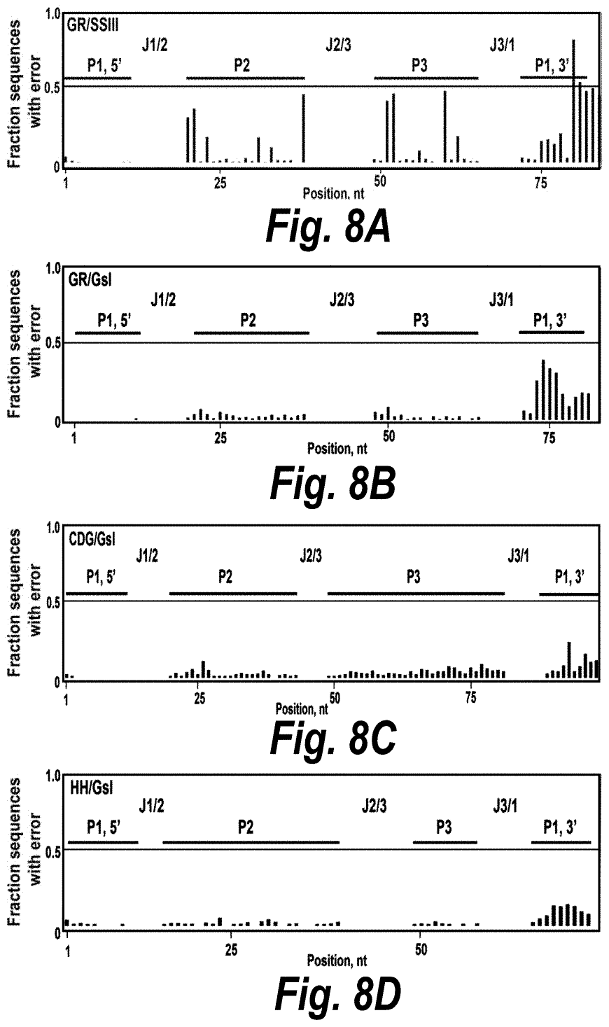

Unexpectedly, in addition to the limited sequence diversity of the SSIII selection, a heavy accumulation of deletions and point mutations was observed such that no sequences retaining the full identity of the constant regions of the scaffold were recovered in the final round. Two of the clusters, 5HTP-I and 5HTP-III (FIG. 2D), have deletions in L2 or L3 of the scaffold essential to the formation of the loop-loop interaction of purine riboswitches. In addition, 5HTP-III members contain several point deletions acquired during the selection that yields the potential for a drastically alternative secondary structure. Minimal free energy (MFE) and covariation analysis of this sequence suggest a secondary structure consistent with the consensus sequence of the L-tryptophan aptamer (an aptamer that comprises a two-way junction; Majerfeld & Yarus, Nucleic Acids Research, 2005, 33, 5482-5493), further suggesting that the scaffold was not maintained in the 5HTP-III family. 5HTP-II is the only major cluster maintaining sequence requirements necessary for the tertiary structure designed into the library and is the only abundant sequence shared between the SSIII and GsI selections. In contrast to the selection using SSIII, the GsI selections exhibited low amounts of mutations accruing in the scaffold's constant region, indicating robust maintenance of the scaffold (FIG. 8).

To identify sequences with high aptamer potential, the ten most populous clusters from each pool were individually aligned, MFE structures predicted, and covariation models generated. This allowed for an information-rich view of the major consensus sequences presented by the selections. Since the GR/SSIII experiment was highly overselected, the most abundant sequence from each of the three major clusters was chosen for further validation. For the GsI selections, the dominant sequences from one or more clusters whose consensus MFE structure was consistent with the parent scaffold were selected.

### Example 3: Most Populated Clusters Preserve Scaffold Architecture and Bind 5HTP With High Selectivity

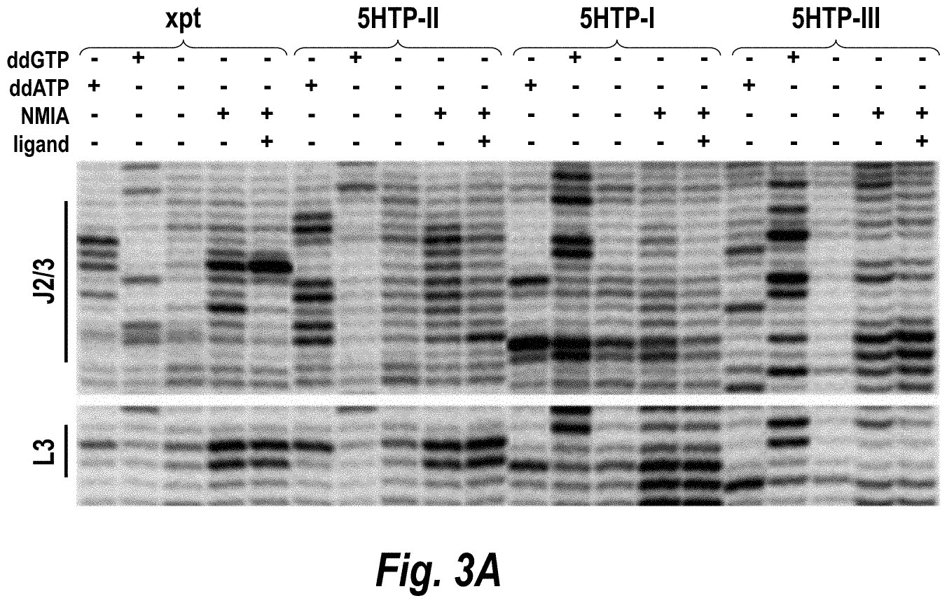

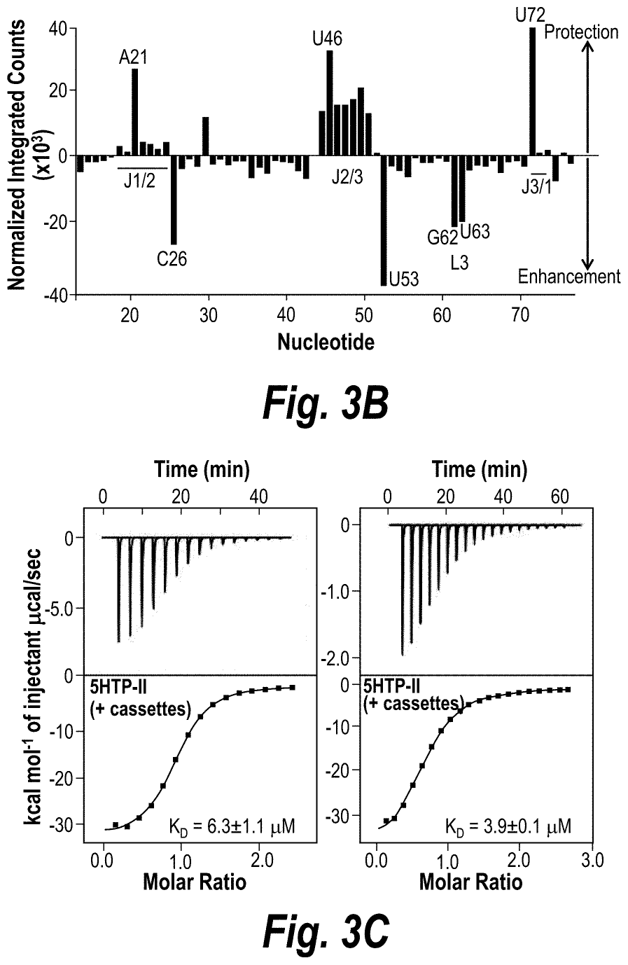

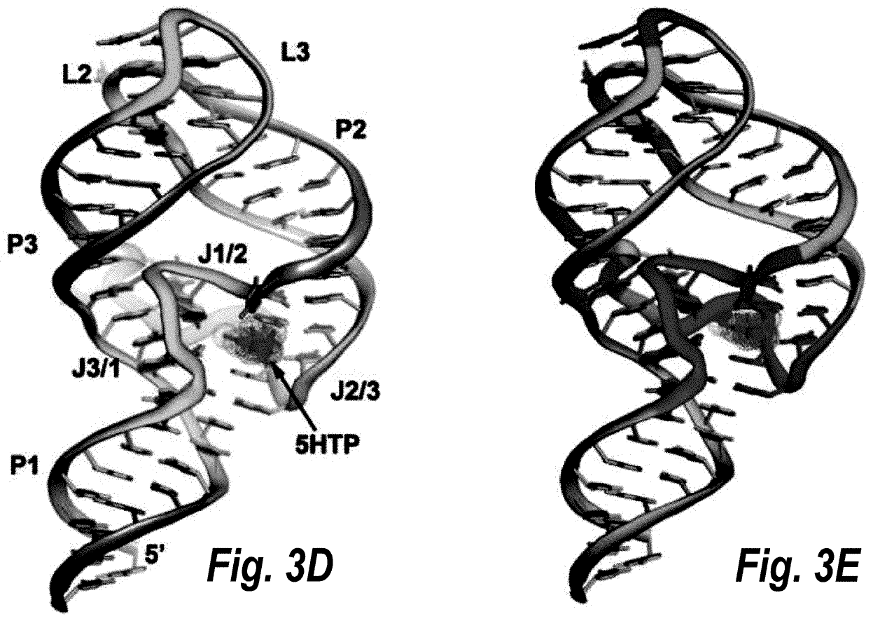

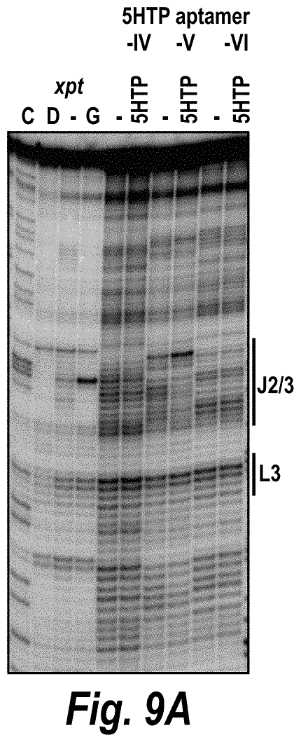

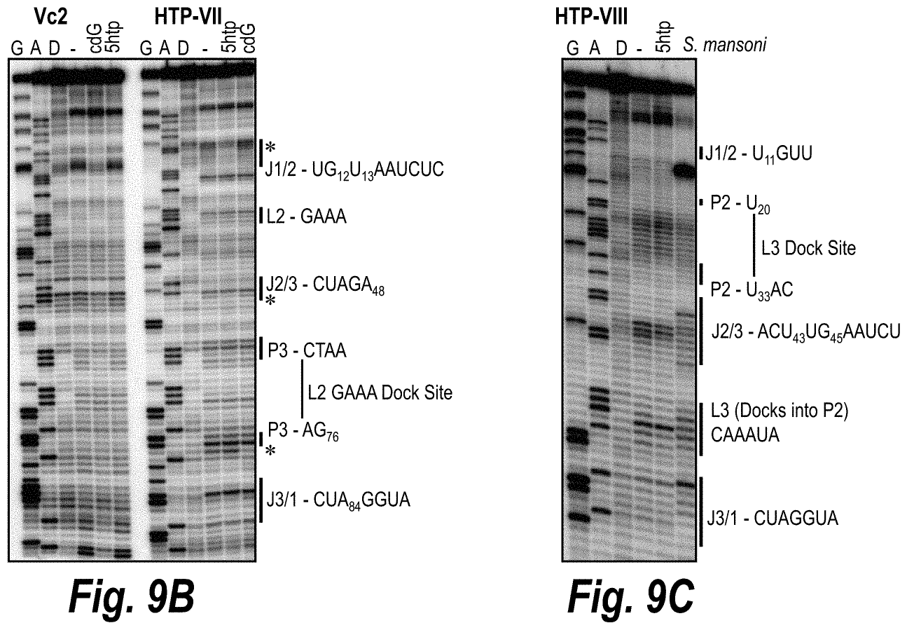

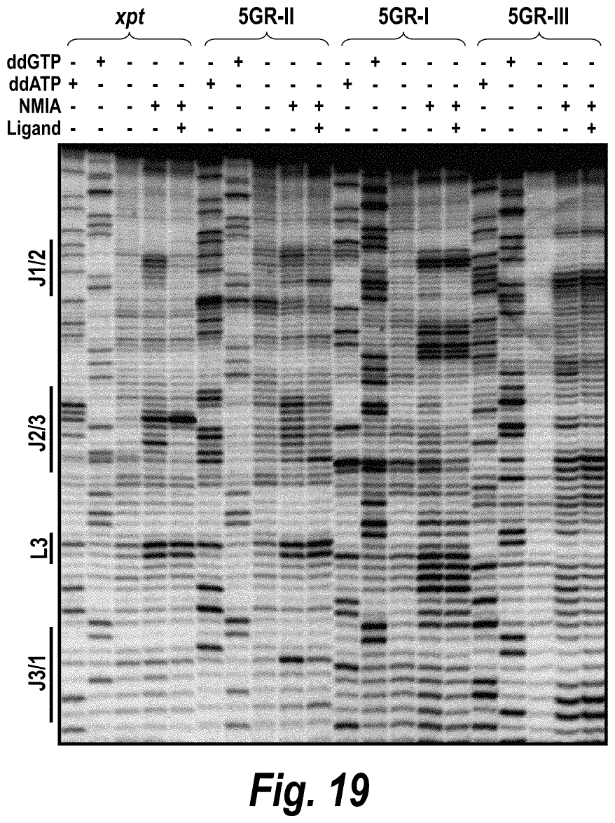

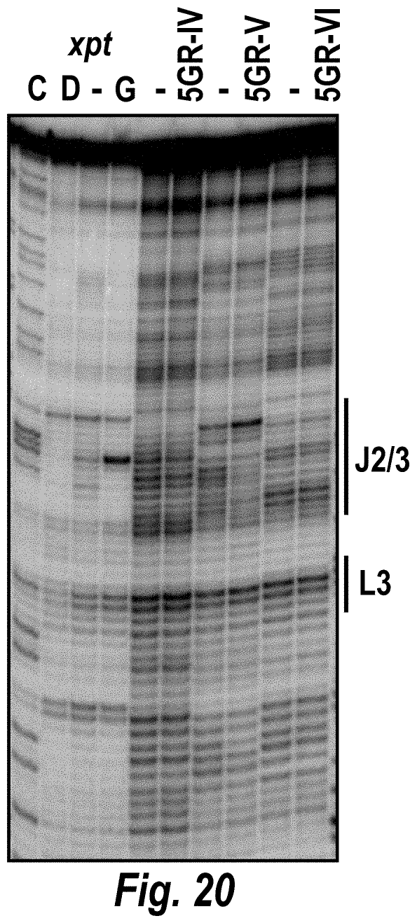

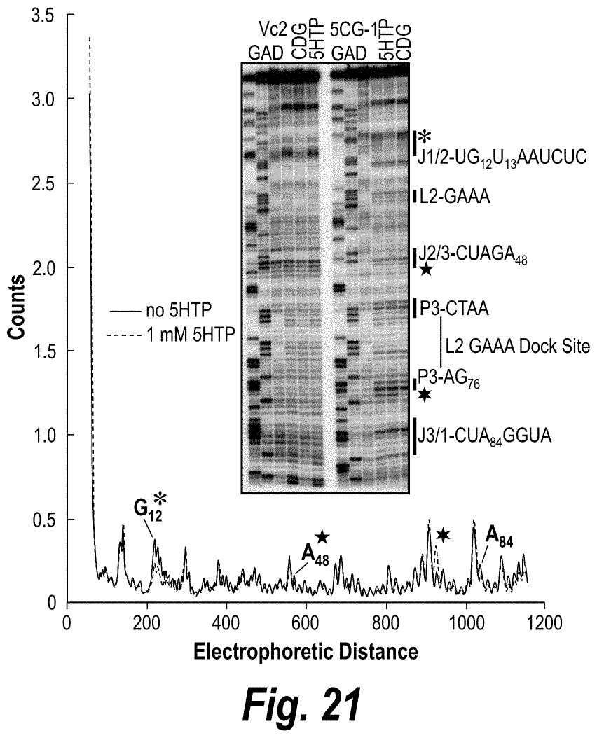

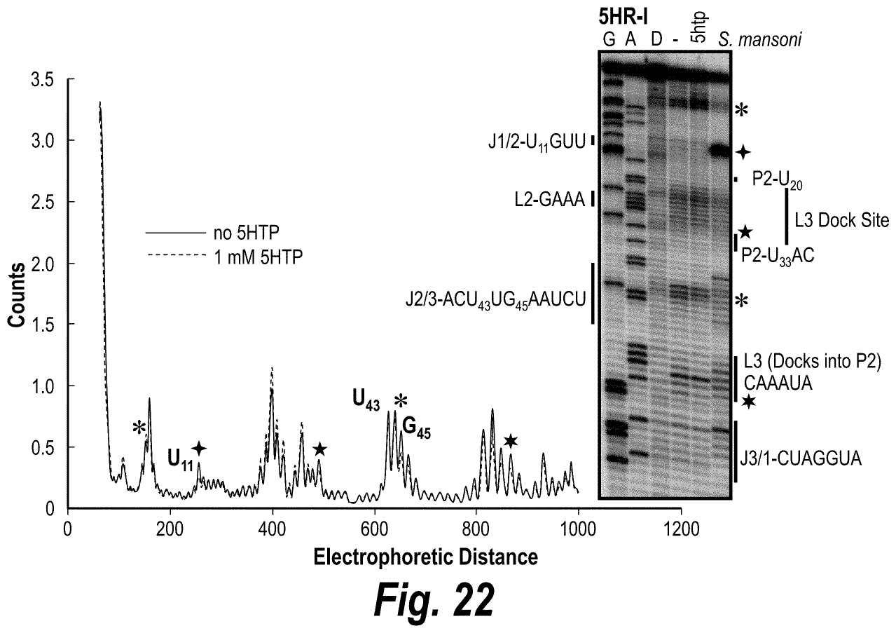

The structural scaffold greatly facilitates validation of the structural and interaction features of resultant aptamers. Chemical probing of RNA structure using N-methylisatoic anhydride (“NMIA”), a technique referred to as “SHAPE,” reveals whether the secondary and tertiary architecture of the parental scaffolds were preserved as well as ligand-dependent structural changes in the aptamer. In the GR/SSIII selection, the 5HTP-I and 5HTP-II aptamers have localized changes in the NMIA reactivity patterns in the presence of ligand within the three-way junction elements, consistent with this being the ligand-binding site (FIG. 3A, FIG. 19). 5HTP-III, however, shows changes outside of J2/3 in the constant regions, consistent with the predicted structure and L-Trp binding site of a previously described tryptophan aptamer (Majerfeld & Yarus). Preservation of the GR scaffold was assessed using a unique, ligand-independent NMIA reactivity signature in L3 that is only present when it interacts with L2 (Stoddard et al., RNA, 2008, 14, 675-684). 5HTP-II is the only sequence from the three clusters of the SSIII selection exhibiting this feature. Conversely, all tested sequences from the GR/GsI selection have this tertiary structure signature (FIGS. 9A and 20). These data strongly indicate that the GR/SSIII selection yielded three distinct aptamers with only 5HTP-II preserving the structural scaffold while the GR/GsI selection produced multiple solutions maintaining the scaffold. While the 5HTP-dependent signatures for the GR/GsI isolates are weaker than those of 5HTP-II and the parental aptamer, quantification reveals that they localize to the junction in an analogous manner (FIG. 3B). SHAPE characterization of the CDG/GsI and HH/GsI selections shows ligand-dependent changes for the new classes of aptamers and an overall reactivity pattern resembling the parental scaffold (FIGS. 9B, 9C, 21 and 22).

The affinity and selectivity of these aptamers for 5HTP and a set of chemically similar compounds was assessed by isothermal titration calorimetry (ITC). Importantly, for all of the tested aptamers, the 5′- and 3′-cassette sequences were not required for 5HTP binding, indicating the successful design of neutral sequences (FIG. 3C). Several trends emerged from this analysis. First, both aptamers that do not preserve the parent scaffold (5HTP-I, -III) do not discriminate between 5HTP and L-tryptophan (Table 3), a crucial requirement for cell-based applications. Second, the majority of the aptamers preserving the three-way junction scaffold have higher affinities for 5HTP than the aptamers with disrupted scaffolds and all strongly discriminate against L-tryptophan. This indicates that the architecture of the scaffold is important for creating a selective binding pocket while maintaining affinities comparable to other synthetic and natural aptamers that bind amino acids. 5HTP-I and 5HTP-III show strong discrimination between 5HTP and serotonin, implying that main chain atoms are directly recognized. In contrast, many of the aptamers that preserve the scaffold bind N-methyl-5-hydroxy-L-tryptophanomide with 2- to 4-fold higher affinity than 5HTP. Also, they bind serotonin, the decarboxylation product of 5HTP, suggesting a lesser requirement for the main chain atoms in binding (Table 3). Thus, a few of these aptamers could be outstanding serotonin sensors. Most striking from the GsI selections is that the dominant aptamers from each selection, despite having different scaffold architectures, converged on highly similar binding affinities and selectivity profiles, revealing that three-way junctions are a robust fold for hosting 5HTP binding pockets. Together, these data show that distinct oligonucleotide junctions, such as three-way junction architectural variants, are able to find robust solutions for 5HTP recognition.

TABLE 3
Affinity of aptamers for 5HTP and related compounds

| | | 5HTP | L-Trp | Serotonin | Me-5HTP |
|---|---|---|---|---|---|
| Selection | sequence | KD, μM^{a} | KD, μM | KD, μM | KD, μM |
| GR/SSIII | 5HTP-I | 33 ± 1 | 41 ± 1 | >1000 | — |
| | 5HTP-II | 3.9 ± 0.1 | 280 ± 30 | 38 ± 8 | 26 ± 9 |
| | 5HTP-III | 38 ± 14 | 20 ± 5 | >1000 | — |
| GR/GsI | 5HTP-IV | 8.8 ± 1.5 | 520 ± 90 | 4.7 ± 0.3 | 1.3 ± 0.1 |
| | 5HTP-V | 11 ± 1 | 170 ± 10 | 16 ± 4 | 6.6 ± 0.4 |
| | 5HTP-VI | 60 ± 15 | N.D.^{b} | 16 ± 2 | 25 ± 8 |
| CDG/GsI | 5HTP-VII | 9.3 ± 0.3 | N.D. | 1.2 ± 0.1 | 2.1 ± 0.4 |
| HH/GsI | 5HTP-VIII | 7.3 ± 2.8 | N.D. | 1.2 ± 0.2 | 2.5 ± 0.5 |

^{a}All measurements taken at 25° C. in 10 mM MgCl₂ containing buffer.
^{b}Not detectable.

### Example 4: Structural Analysis of 5GR-II Aptamer Reveals a Recurrent RNA Motif is Used for 5HTP Binding

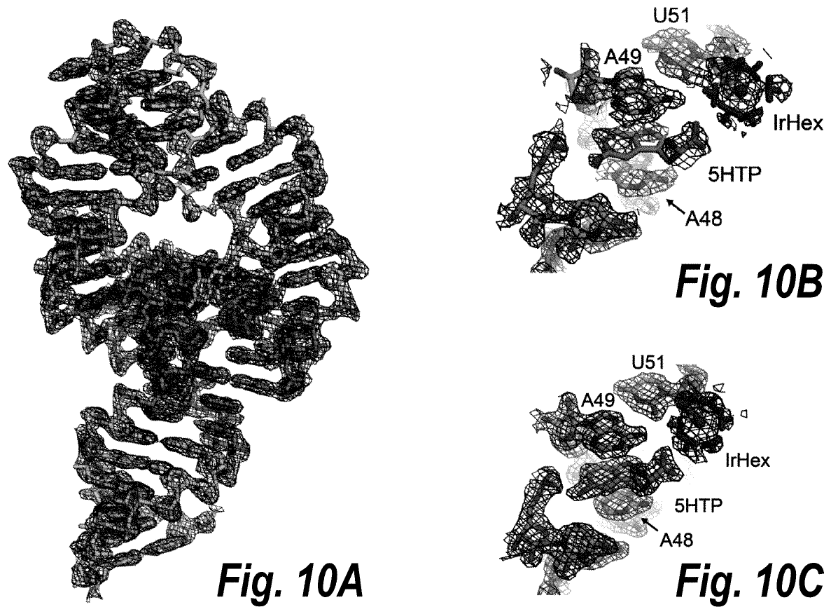

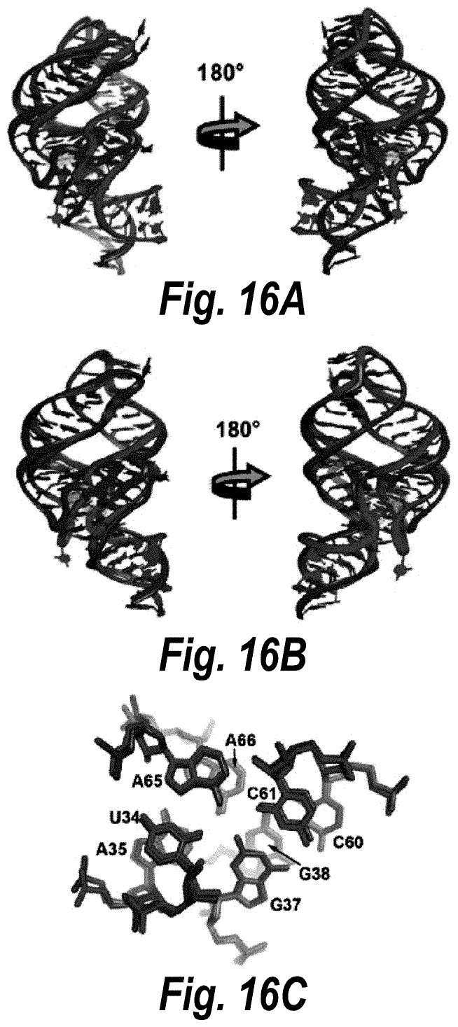

To further demonstrate that the scaffolded selection strategy described herein preserved the fold of the parental RNA and to elucidate how RNA can recognize 5HTP, the structure of 5HTP-II complexed with 5HTP was determined at 2.0 A resolution (FIG. 3D, representative electron density maps are shown in FIG. 10 and crystallographic statistics are given in Table 4). This structure globally superimposes with the parental xpt guanine riboswitch aptamer with an r.m.s.d. of 6.5 Å over all backbone atoms in residues 19-77, with the main sources of deviation produced by a different angle for P1 in relation to the binding pocket and the varied junction region (FIGS. 16A-C). Within the L2-L3 tertiary interaction the pattern of base-base interactions and backbone geometry is almost identical between the two RNAs (r.m.s.d. 0.96 Å over all atoms in residues 31-39, 61-67). Thus, the GR scaffold remained intact, both globally and locally, during the selection process.

TABLE 4
Crystallographic data and refinement statistics.

| | 5HTP-II RNA/5HTP |
|---|---|
| Data collection | |
| Space group | C121 |
| Cell dimensions | |
| a, b, c (Å) | 127.55, 26.59, 63.37 |
| α, β, γ (°) | 90, 106.32, 90 |
| Resolution (Å) | 19.95-2.00 (2.07-2.00)* |
| R_{sym} or R_{merge} | 0.084 (0.191) |
| I \|σI | 11.2 (5.4) |
| Completeness (%) | 96.2 (73.8) |
| Redundancy | 4.34 (3.65) |
| Refinement | |
| Resolution (Å) | 18.33-2.00 (2.07-2.00) |
| No. unique reflections | 13,725 |
| R_{work} / R_{free} | 21.7/25.8 (20.1/26.0) |
| No. atoms | |
| RNA | 1513 |
| Ligand/ion | 16/90 |
| Water | 112 |
| B-factors (average) | |
| RNA | 29.5 |
| Ligand/ion | 16.2/35 |
| Water | 23.5 |
| r.m.s. deviations | |
| Bond lengths (Å) | 0.007 |
| Bond angles (°) | 1.308 |

*Values in parentheses are for the highest-resolution shell.

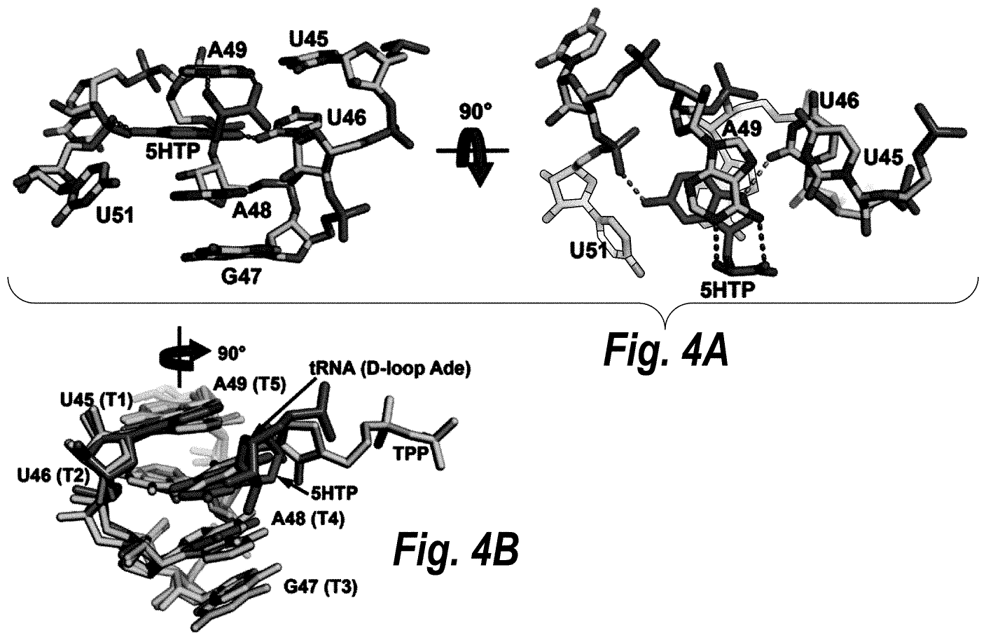

The ligand binding pocket of 5HTP-II resides within the three-way junction that has a radically different local structure from the parent RNA. Direct ligand contacts are primarily mediated by nucleotides in J2/3 using a common RNA structural module, the T-loop (FIG. 4A). The first five nucleotides of J2/3 form a canonical T-loop structure superimposing almost perfectly with a tRNA^{Phe} T-loop (r.m.s.d. 0.49 Å for backbone residues). Stabilization of position 3 in the tRNA T-loop by long range Watson-Crick pairing with the D-loop is critical for activity. 5HTP-II possesses a similar interaction between G47 of the T-loop and C75 of J3/1. The 5HTP-II T-loop hosts 5HTP stacked between positions 4 and 5 in a manner orthologous to how the tRNA T-loop hosts an intercalating purine from the D-loop and is also similar to thiamine pyrophosphate (TPP) recognition by its riboswitch (FIG. 4B). While the T-loop is directly responsible for recognition of the ligand, nucleotides from all three randomized regions are involved in local structure aiding in the formation of a compact junction that stabilizes the T-loop. Given such a complex set of interactions supporting the T-loop, it is unlikely that the isolated T-loop binds 5HTP.

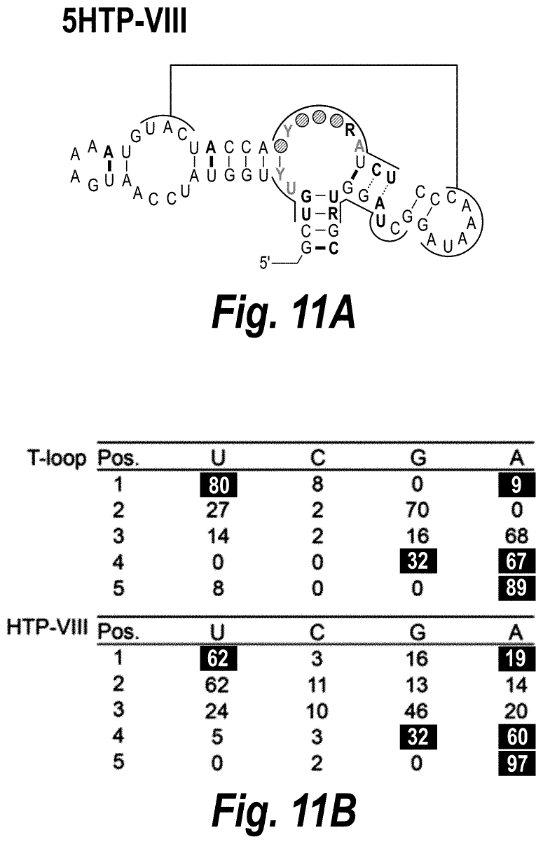

The crystal structure of 5HTP-II yields additional insights into 5HTP recognition by the other scaffolded aptamers. The most abundant cluster in the GR/GsI selection, 5HTP-IV, also contains the UUGAA signature of the T-loop. The motif, however, is 3′-shifted by a single nucleotide, likely leading to an alternative orientation within the three-way junction as suggested by significant sequence differences in J1/2 and J3/1 between 5HTP-II and 5HTP-IV. In the HH selection, the most abundant sequence of the most populous cluster (5HTP-VIII) also contains the conserved UUGAA sequence of the T-loop in J2/3. Sequence variation analysis of this region of the 5HTP-VIII aptamer reveals a pattern of conservation matching that of the biological T-loops with only slight deviations (FIG. 11). This suggests that the T-loop motif may be a robust module for the recognition of small planar compounds by RNA. While there is no clearly identifiable T-loop in RNAs from the CDG selection, the binding parameters almost perfectly match that of the other two selections, suggesting a similar recognition mode.

### Example 5: Scaffolded Aptamers Can Be Readily Incorporated Into Robust Small Molecule Sensory Devices

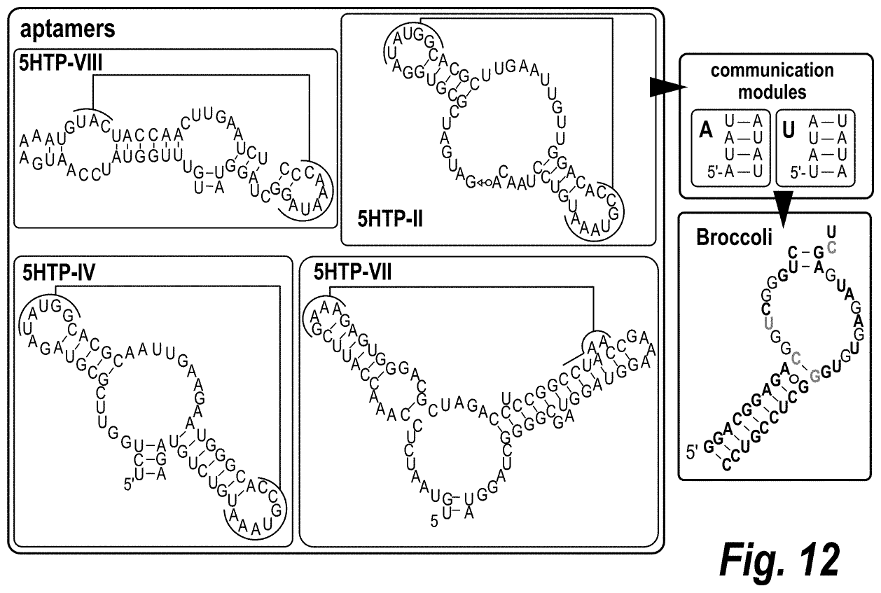

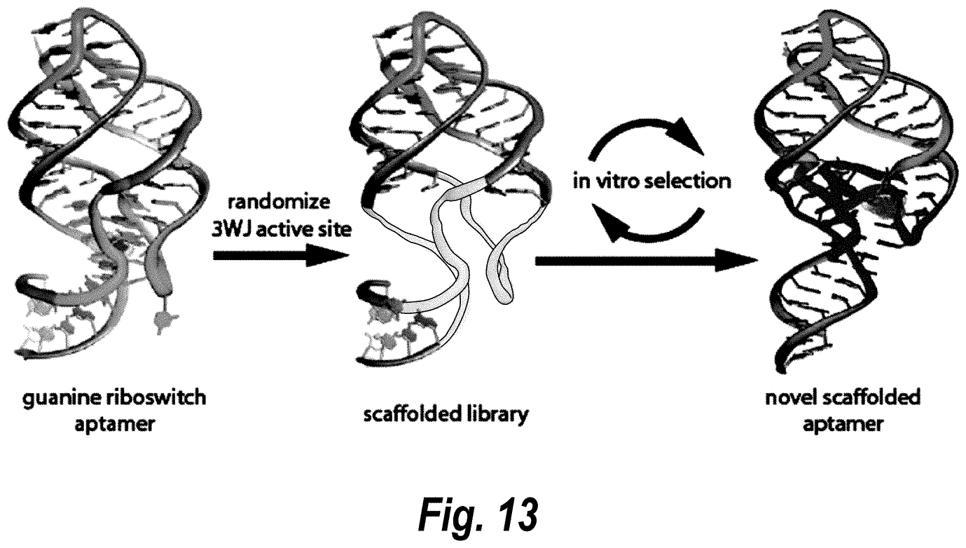

With scaffolded selection techniques proving capable in creating well folded, highly structured, and specific RNA aptamers, its ability to produce functional synthetic RNA biosensors was tested. To create these devices, a strategy of linking a small molecule binding aptamer to a fluorophore-binding module via a short helical element was used. The lead candidate aptamer from each library was coupled to the Broccoli fluorophore binding aptamer with two helical variants (the communication modules are referred to as “A” and “U”, FIG. 12) linking the two aptamers. This resulted in a set of RNAs capable of sensing 5HTP and/or serotonin over several orders of magnitude in vitro with varying output fluorescence dynamic ranges (Table 5 and Table 6). Many of these sensors, when in the presence of ligand, are capable of producing fluorescence levels equal to or greater than that of the unconjugated broccoli aptamer alone under identical conditions. Inherent to this system is a reduced apparent F₅₀ (defined as the ligand concentration required to elicit a half maximal fluorescent response) relative to the K_{D} of the isolated aptamer as monitored by the Broccoli fluorescence. However, several scaffolded aptamers show only a ˜10-fold difference between their K_{D} and F₅₀. Overall, this compares favorably to examples of natural riboswitch aptamer domains in the literature whose differences in K_{D} and F₅₀ can approach 1000-fold, an important trait to consider when sensitivity or ligand toxicity is a limiting factor in riboswitch application.

TABLE 5
In vitro performance of Broccoli based 5HTP/serotonin sensors

| | linker/ligand | | | |
|---|---|---|---|---|
| | A/5HTP^{a} | A/5HT | U/5HTP | U/5HT |
| aptamer | F₅₀, μM | F₅₀, μM | F₅₀, μM | F₅₀, μM |
| 5HTP-II | 190 ± 30 | N.D.^{b} | 180 ± 20 | N.D. |
| 5HTP-IV | N.D. | 190 ± 70 | 240 ± 20 | 52 ± 4 |
| 5HTP-VIII | N.D. | 790 ± 190 | 590 ± 90 | 260 ± 50 |

^{a}All measurements taken at 25° C. in 5 mM MgCl₂ containing buffer.
^{b}Not detectable.

TABLE 6
Performance of 5HTP-Broccoli sensors

| | | | Fold | Fₘₐₓ | |
|---|---|---|---|---|---|
| Sensor | Ligand | [MgCl₂], mM | Induction^{a} | (% Broccoli)^{b} | F₅₀^{c}, μM |
| 5HTP-II/A | 5HTP | 1 | 3.5 | 18.5 | 190 ± 30 |
| | | 3 | 6.3 | 71 | n.d. |
| | | 5 | 7.3 | 122 | |
| | | 10 | 6 | 168 | |
| | | 20 | 4.1 | 180 | |
| | 5HT | 1 | 1.3 | 6.9 | |
| | | 3 | 3.1 | 34.6 | |
| | | 5 | 3.7 | 62.7 | |
| | | 10 | 3.9 | 108 | |
| | | 20 | 3 | 134 | |
| 5HTP-II/U | 5HTP | 1 | 1.9 | 21.1 | 180 ± 20 |
| | | 3 | 3.1 | 72.2 | n.d. |
| | | 5 | 3.3 | 105 | |
| | | 10 | 3.3 | 130 | |
| | | 20 | 3.2 | 135 | |
| | 5HT | 1 | 0.4 | 4.7 | |
| | | 3 | 0.8 | 18.2 | |
| | | 5 | 1.1 | 33.2 | |
| | | 10 | 1.3 | 52.6 | |
| | | 20 | 1.6 | 66.4 | |
| 5HTP-IV/A | 5HTP | 1 | 1.2 | 2.7 | n.d. |
| | | 3 | 1.3 | 2.9 | 190 ± 70 |
| | | 5 | 1.4 | 3.1 | |
| | | 10 | 1.7 | 3.8 | |
| | | 20 | 2 | 4.3 | |
| | 5HT | 1 | 1.0 | 2.4 | |
| | | 3 | 1.3 | 2.75 | |
| | | 5 | 1.4 | 3.2 | |
| | | 10 | 1.8 | 4 | |
| | | 20 | 2.2 | 4.7 | |
| 5HTP-IV/A | 5HTP | 1 | 2.5 | 8.5 | 240 ± 20 |
| | | 3 | 6 | 37.1 | 52 ± 4 |
| | | 5 | 8.2 | 71.2 | |
| | | 10 | 8.2 | 111 | |
| | | 20 | 6.1 | 126 | |
| | 5HT | 1 | 5.1 | 17.2 | |
| | | 3 | 8.8 | 54.1 | |
| | | 5 | 9 | 78.1 | |
| | | 10 | 7.2 | 98.3 | |
| | | 20 | 5.1 | 105 | |
| 5HTP-VIII/A | 5HTP | 1 | 1.1 | 3.7 | 790 ± 190 |
| | | 3 | 1.8 | 7 | |
| | | 5 | 2.4 | 10.4 | |
| | | 10 | 3.7 | 18 | |
| | | 20 | 5.1 | 26.9 | |
| | 5HT | 1 | 1.5 | 5 | |
| | | 3 | 4.4 | 17.7 | |
| | | 5 | 7.2 | 31.4 | |
| | | 10 | 10.9 | 53.3 | |
| | | 20 | 13 | 69.5 | |
| 5HTP-VIII/U | 5HTP | 1 | 1.6 | 12.3 | 590 ± 90 |
| | | 3 | 2.5 | 43.3 | 260 ± 50 |
| | | 5 | 2.8 | 69.7 | |
| | | 10 | 3.1 | 109 | |
| | | 20 | 3 | 126 | |
| | 5HT | 1 | 3.8 | 30 | |
| | | 3 | 5.5 | 93.4 | |
| | | 5 | 4.8 | 116 | |
| | | 10 | 3.7 | 131 | |
| | | 20 | 3.2 | 136 | |

^{a}Defined as (fluorescence at saturating ligand/fluorescence in absence of ligand); grey shading denotes sensors that showed strong performance.
^{b}Defined as (maximum sensor fluorescence/isolated broccoli aptamer fluorescence)*100.
^{c}Defined as the concentration of ligand required to elicit the half maximal fluorescence response.

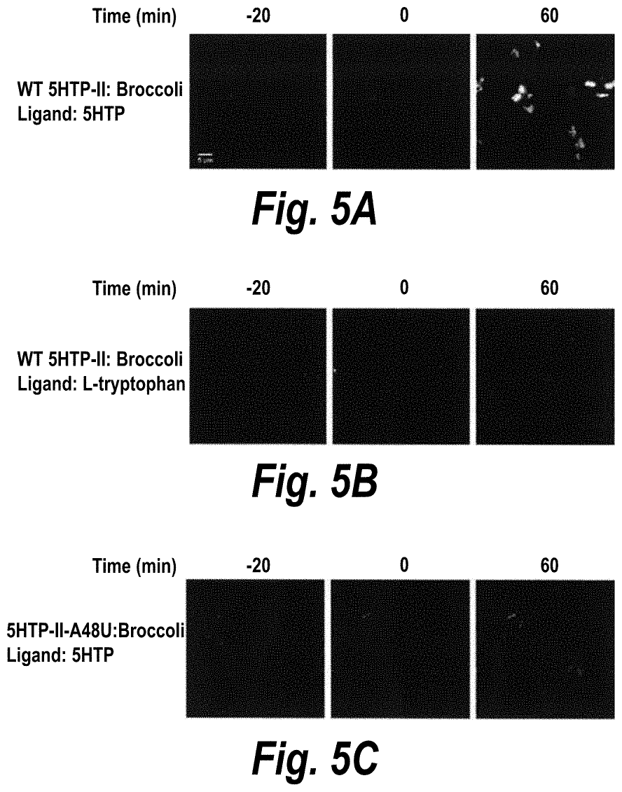

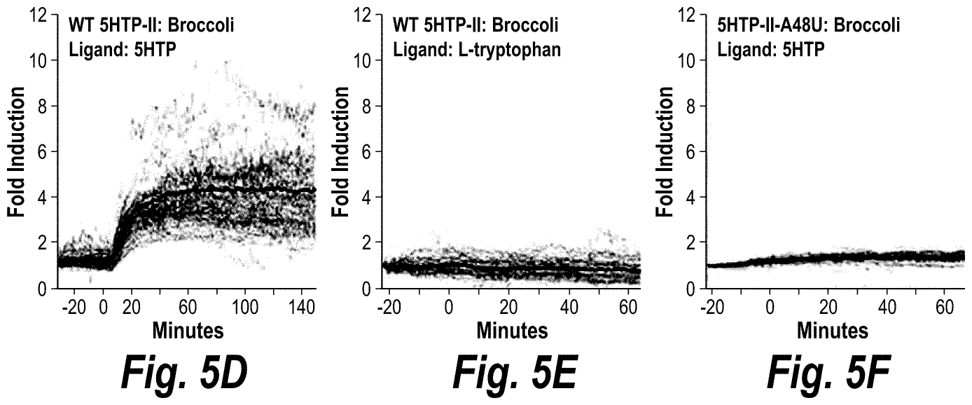

Of the above devices, 5HTP-II(A) is capable of specifically sensing 5HTP in E. coli. This genetically encoded sensor yielded a rapid induction of fluorescence upon addition of 2 mM 5HTP to E. coli growing in a rich chemically defined medium (10 minutes), with approximately 80% of bacteria displaying an observable response within 20 minutes (FIG. 5). The fluorescence signal was completely dependent upon the RNA device binding 5HTP. No signal gain was observed when L-tryptophan was included in the media or when the sensor contained a point mutation (A48U) in the T-loop module that ablated ligand binding to the isolated aptamer (data not shown). Furthermore, the increase in relative fluorescence in the presence of 5HTP was comparable to robust cyclic dinucleotide sensors based upon natural riboswitch aptamer domains in live cells. Importantly, these observations are in contrast to claims that non-natural aptamers have reduced intracellular performance compared to natural aptamers in the context of fluorometric sensors (You, PNAS (2015) 112:21, E2756-2765).

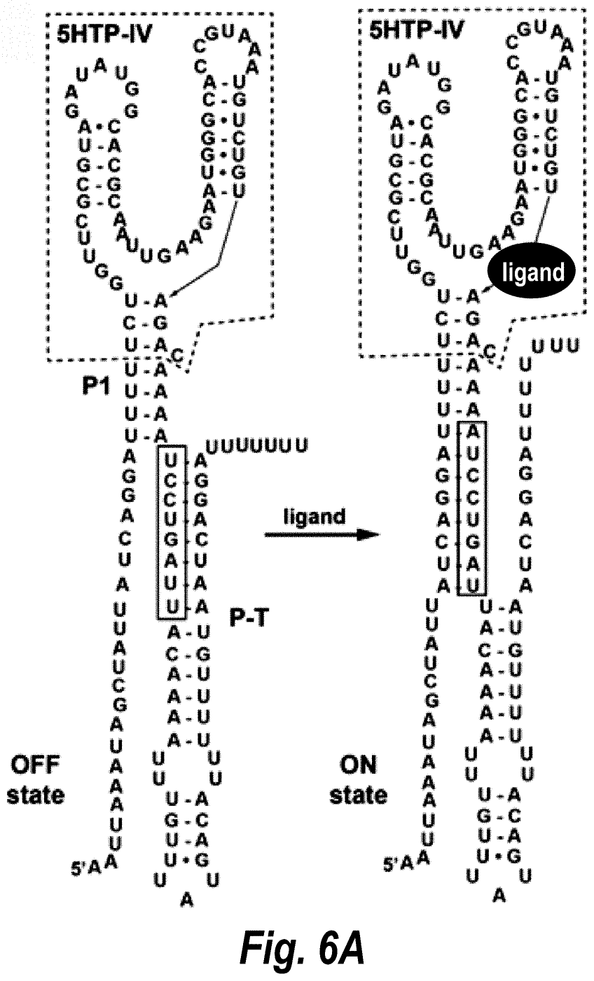

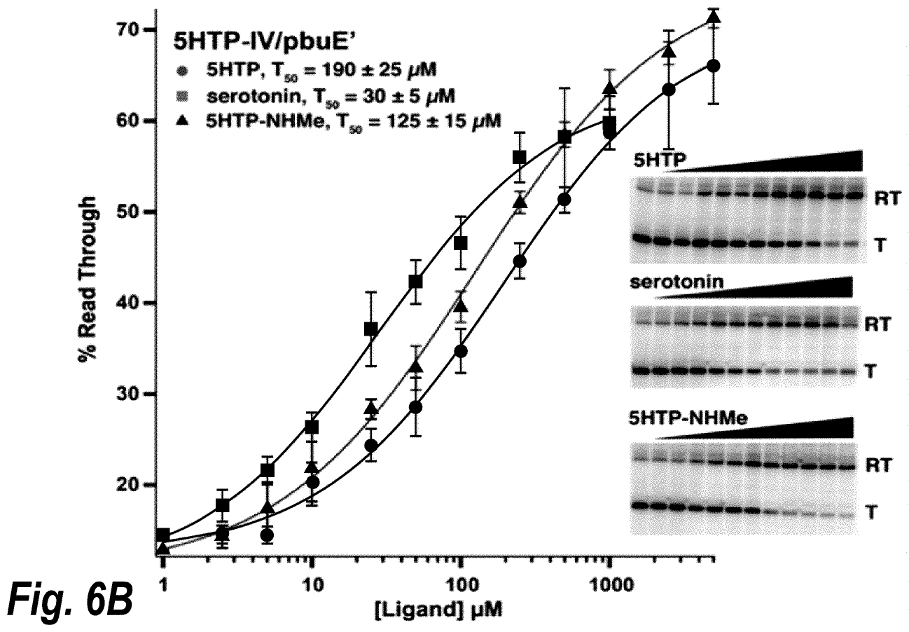

Select scaffolded 5HTP aptamers were also coupled to engineered modular secondary switches derived from natural riboswitch expression platforms to generate gene regulatory elements. Using a coupling strategy in which the P1 helix of the aptamer and expression platform is directly coupled, a proficient ligand-dependent regulator of transcription was engineered by fusing the 5HTP-IV sensor and pbuE “ON” switch platform (FIG. 6A). The resulting RNA element is capable of activating transcriptional read-through in vitro with a specificity profile identical to the aptamer domain in isolation and possesses a dynamic range consistent with natural riboswitches (FIG. 6B); surprisingly, L-Trp is completely incapable of enabling read-through transcription. Again, the discrepancy between K_{D} and T₅₀ was not insignificant (6-fold for serotonin, 22-fold for 5HTP), but reflected observed trends for natural riboswitches where an aptamer's thermodynamic properties do not always dictate its ability to communicate with an adaptor sequence.

### Example 6: Scaffolded Aptamers According to Exemplary Embodiments

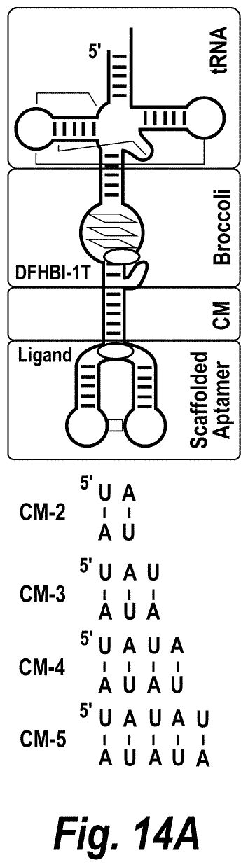

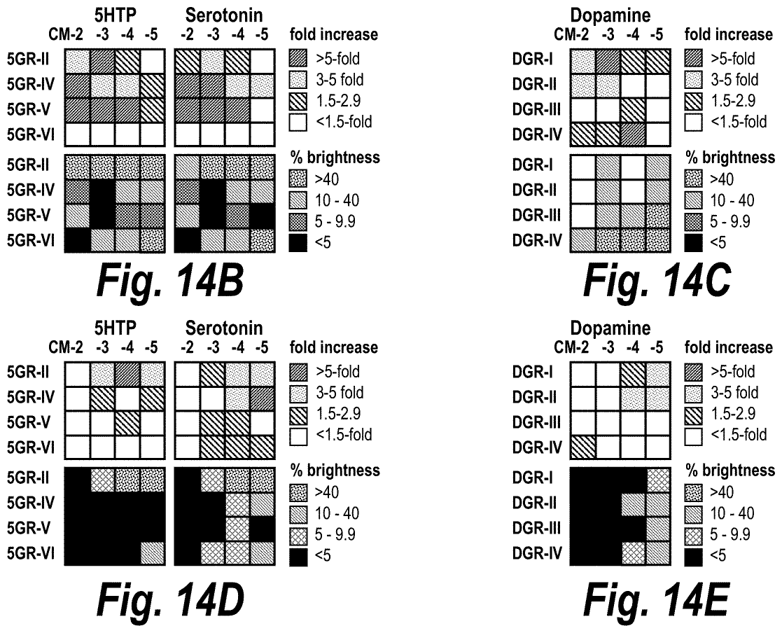

The Broccoli aptamer was coupled to a tRNA scaffold to stabilize the biosensor for cell-based applications. Four different GR-scaffolded 5HTP aptamers were coupled to four communication modules of differing lengths (two to five A-U and U-A base pairs; FIG. 14A) and each resultant biosensor tested for the ability to fluoresce in a ligand-dependent fashion. Each sensor was assessed for their ligand-dependent fold change in fluorescence and maximal brightness relative to the isolated Broccoli aptamer both in vitro (FIG. 14B; Tables 7 and 8) and in E. coli (FIG. 14D; Tables 9 and 10). To enable rapid screening of candidates in vitro, the biosensors were transcribed and directly used in the fluorometric assay without further purification. These data reveal three aptamers (5GR-II, -IV, and -V) yielded sensors that can detect 5HTP and/or serotonin both in vitro and in the cellular context, with 5GR-II demonstrating the best performance with respect to combined fold increase in fluorescence and maximal brightness.

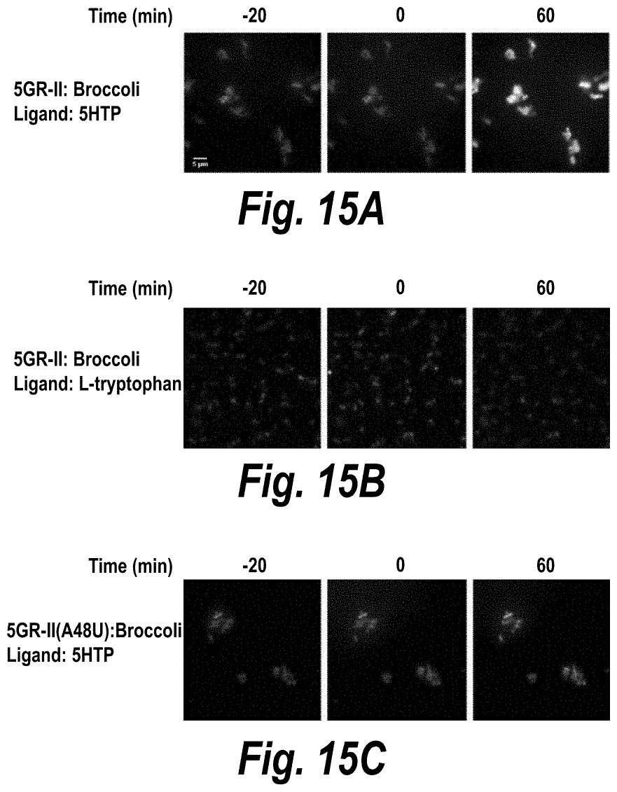

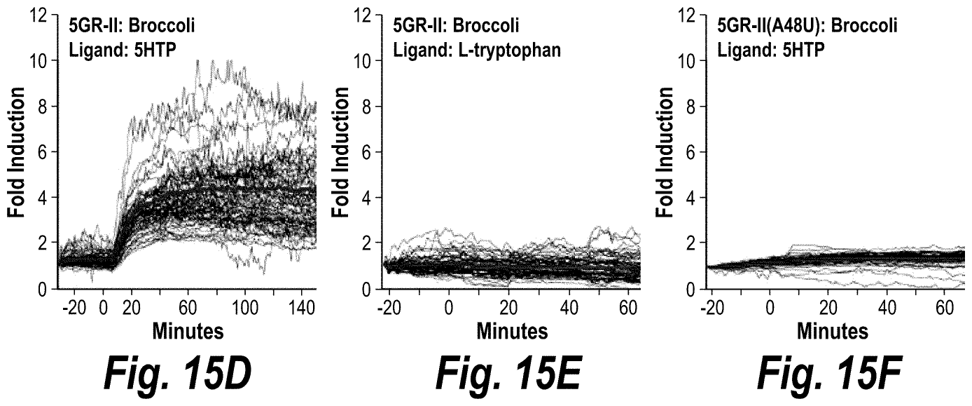

To further demonstrate the potential of scaffolded aptamers, live cell imaging was used to visualize the uptake of 5HTP by E. coli using the 5GR-II/CM-4 biosensor. Fluorescence imaging of single cells revealed a rapid induction of fluorescence upon addition of 2 mM 5HTP to E. coli growing in a rich chemically defined medium, with approximately 80% of bacteria displaying an observable response within 20 minutes (FIGS. 15A, D). The fluorescence signal was completely dependent upon the RNA device binding 5HTP; no detectable signal gain was observed when L-tryptophan was included in the media (FIGS. 15B, E) or when the sensor contained a point mutation (A48U) in the T-loop module that ablated ligand binding to the isolated aptamer (FIGS. 15C, F). The observed increase in relative fluorescence in the presence of 5HTP was comparable to robust cyclic dinucleotide sensors based upon natural riboswitch aptamer domains in live cells. These results contrast previous claims that synthetic aptamers have reduced intracellular performance compared to natural aptamers and it is shown here that multiple synthetic aptamers are capable of functioning within E. coli in the context of an allosteric fluorogenic RNA.

The above 5HTP biosensors were designed with knowledge from biochemical and biophysical analysis of select aptamers. However, an optimal workflow for rapid development of biosensors would be able to use information derived only from the computational analysis of the selection to design candidate RNAs. To demonstrate that scaffolded aptamers incorporate design principles that enable biosensor engineering in the absence of experimental characterization, the above biosensor strategy was employed for four aptamers derived from the L-DOPA selection. None of these aptamers were validated in any fashion prior to their incorporation into allosteric fluorogenic sensors. Screening of the resultant biosensors with L-DOPA and dopamine in vitro (FIG. 14C; Tables 7 and 8) and in E. coli (FIG. 14E; Tables 9 and 10) revealed two aptamers (DG-I and DG-II) that function in both contexts.

TABLE 7
In vitro-fold induction of fluorogenic GR-scaffolded aptamers

| aptamer | ligand | CM-2 | CM-3 | CM-4 | CM-5 |
|---|---|---|---|---|---|
| 5GR-II | 5HTP^{a} | 3.5 ± 0.1^{b} | 5.1 ± 0.2 | 2.5 ± 0.1 | 1.2 ± 0.1 |
| 5GR-IV | 5HTP | 14 ± 1 | 3.5 ± 0.7 | 4.1 ± 0.2 | 2.7 ± 0.2 |
| 5GR-V | 5HTP | 13 ± 1 | 11 ± 1 | 7.4 ± 0.4 | 1.8 ± 0.1 |
| 5GR-VI | 5HTP | 0.9 ± 0.1 | 1.2 ± 0.1 | 0.9 ± 0.1 | 1.1 ± 0.1 |
| 5GR-II | serotonin | 1.5 ± 0.1 | 3.7 ± 0.1 | 1.8 ± 0.1 | 1.1 ± 0.1 |
| 5GR-IV | serotonin | 16 ± 1 | 5.5 ± 1.1 | 4.2 ± 0.2 | 3.2 ± 0.2 |
| 5GR-V | serotonin | 11 ± 1 | 8.9 ± 0.8 | 7.3 ± 0.6 | 1.3 ± 0.2 |
| 5GR-VI | serotonin | 0.7 ± 0.1 | 1.4 ± 0.2 | 0.8 ± 0.1 | 1.1 ± 0.1 |
| DGR-I | 3, 4-DHF | 2.4 ± 0.2 | 6.4 ± 0.6 | 2.3 ± 0.2 | 1.5 ± 0.1 |
| DGR-II | 3, 4-DHF | 2.3 ± 0.4 | 3.8 ± 0.8 | 1.2 ± 0.1 | 1.1 ± 0.1 |
| DGR-III | 3, 4-DHF | 1.3 ± 0.1 | 1.4 ± 0.1 | 1.6 ± 0.1 | 1.1 ± 0.2 |
| DGR-IV | 3, 4-DHF | 1.9 ± 0.1 | 1.7 ± 0.1 | 4.3 ± 0.2 | 1.3 ± 0.1 |
| DGR-I | dopamine | 3.5 ± 0.6 | 7.9 ± 0.9 | 2.5 ± 0.2 | 1.5 ± 0.1 |
| DGR-II | dopamine | 4.6 ± 0.8 | 4.2 ± 1.1 | 1.3 ± 0.1 | 1.1 ± 0.1 |
| DGR-III | dopamine | 1.2 ± 0.1 | 1.4 ± 0.1 | 1.7 ± 0.1 | 1.0 ± 0.1 |
| DGR-1V | dopamine | 2.6 ± 0.1 | 2.0 ± 0.1 | 8.0 ± 0.3 | 1.3 ± 0.1 |

^{a}Ligand concentration is 2 mM.
^{b}Fold Induction (FI) is calculated as (total fluorescence, +ligand)/(total fluorescence, −ligand).
Error is reported as the standard error of the mean for three independent experiments.

TABLE 8
In vitro brightness of fluorogenic GR-scaffolded aptamers relative to parental
Broccoli

| aptamer | ligand | CM-2 | CM-3 | CM-4 | CM-5 |
|---|---|---|---|---|---|
| 5GR-II | 5HTP^{a} | 54 ± 10 | 75 ± 14 | 89 ± 16 | 97 ± 20 |
| 5GR-IV | 5HTP | 5.9 ± 1.4 | 0.4 ± 0.1 | 10 ± 2 | 19 ± 4 |
| 5GR-V | 5HTP | 13 ± 3 | 3.4 ± 0.5 | 5.7 ± 1.3 | 5.5 ± 1.0 |
| 5GR-VI | 5HTP | 1.0 ± 0.1 | 12 ± 2 | 14 ± 3 | 43 ± 8 |
| 5GR-II | serotonin | 22 ± 3 | 54 ± 9 | 63 ± 9 | 88 ± 17 |
| 5GR-IV | serotonin | 7 ± 2 | 0.6 ± 0.1 | 11 ± 3 | 23 ± 5 |
| 5GR-V | serotonin | 11 ± 2 | 2.7 ± 0.2 | 6.2 ± 1.3 | 3.8 ± 0.5 |
| 5GR-VI | serotonin | 0.8 ± 0.1 | 13 ± 1 | 12 ± 3 | 45 ± 9 |
| DGR-I | 3, 4-DHF | 0.5 ± 0.1 | 8.9 ± 0.7 | 6.7 ± 1.1 | 22 ± 4 |
| DGR-II | 3, 4-DHF | 1.3 ± 0.7 | 11 ± 4 | 4.7 ± 1.1 | 17 ± 1 |
| DGR-III | 3, 4-DHF | 3.3 ± 0.2 | 25 ± 4 | 15 ± 2 | 61 ± 11 |
| DGR-IV | 3, 4-DHF | 11 ± 3 | 47 ± 8 | 35 ± 5 | 78 ± 10 |
| DGR-I | dopamine | 0.8 ± 0.1 | 12 ± 1 | 7.7 ± 1.6 | 25 ± 6 |
| DGR-II | dopamine | 2.6 ± 1.3 | 12 ± 4 | 5.1 ± 1.0 | 19 ± 2 |
| DGR-III | dopamine | 3.1 ± 0.2 | 25 ± 4 | 16 ± 2 | 56 ± 11 |
| DGR-IV | dopamine | 15 ± 4 | 53 ± 9 | 66 ± 11 | 76 ± 9 |

^{a}Aptamer ligand concentration is 2 mM.
^{b}Percent brightness is calculated as (total fluorescence sensor, +ligand)/(total fluorescence Broccoli, +ligand).
Error is reported as the standard error of the mean for three independent experiments.

TABLE 9
In vivo-fold induction of fluorogenic GR-scaffolded aptamers

| aptamer | ligand | CM-2 | CM-3 | CM-4 | CM-5 |
|---|---|---|---|---|---|
| 5GR-II | 5HTP^{a} | 0.9 ± 0.1 | 2.6 ± 0.3 | 5.3 ± 0.2 | 3.1 ± 0.45 |
| 5GR-IV | 5HTP | 1.0 ± 0.2 | 1.7 ± 0.7 | 1.4 ± 0.15 | 1.9 ± 0.55 |
| 5GR-V | 5HTP | 1.2 ± 0.2 | 1.4 ± 0.2 | 1.6 ± 0.2 | 0.9 ± 0.1 |
| 5GR-VI | 5HTP | 0.9 ± 0.1 | 1.1 ± 0.1 | 1.1 ± 0.1 | 1.4 ± 0.1 |
| 5GR-II | serotonin | 1.0 ± 0.1 | 1.8 ± .1 | 3.0 ± 0.4 | 2.5 ± 0.3 |
| 5GR-IV | serotonin | 1.0 ± 0.1 | 0.9 ± 0.1 | 2.6 ± 0.7 | 6.9 ± 1.2 |
| 5GR-V | serotonin | 1.1 ± 0.1 | 1.7 ± 0.4 | 1.8 ± 0.2 | 0.7 ± 0.1 |
| 5GR-VI | serotonin | 1.0 ± 0.2 | 1.6 ± 0.1 | 1.6 ± 0.1 | 1.9 ± 0.2 |
| DGR-I | dopamine | 0.7 ± 0.1 | 0.9 ± 0.3 | 1.6 ± 0.6 | 2.7 ± 0.5 |
| DGR-II | dopamine | 0.6 ± 0.1 | 1.0 ± 0.4 | 3.1 ± 0.8 | 2.9 ± 0.5 |
| DGR-III | dopamine | 0.6 ± 0.1 | 0.4 ± 0.2 | 0.7 ± 0.1 | 0.8 ± 0.1 |
| DGR-IV | dopamine | 1.7 ± 0.9 | 0.7 ± 0.2 | 1.3 ± 0.1 | 0.9 ± 0.5 |

^{a}Ligand concentration is 2 mM.
^{b}Fold induction (FI) is calculated as (total fluorescence, +ligand)/(total fluorescence, −ligand).
Error is reported as the standard error of the mean for three independent experiments.

Table 10
In vivo brightness of fluorogenic GR-scaffolded aptamers relative to
parental Broccoli aptamer

| aptamer | ligand | CM-2 | CM-3 | CM-4 | CM-5 |
|---|---|---|---|---|---|
| 5GR-II | 5HTP^{a} | 0.4 ± 0.1 | 2 ± 0.3 | 20 ± 2 | 27 ± 2 |
| 5GR-IV | 5HTP | 0.4 ± 0.1 | 0.7 ± 0.4 | 0.7 ± 0.2 | 0.9 ± 0.3 |
| 5GR-V | 5HTP | 0.5 ± 0.1 | 0.4 ± 0.1 | 0.8 ± 0.1 | 0.8 ± 0.1 |
| 5GR-VI | 5HTP | 0.4 ± 0.1 | 0.7 ± 0.1 | 0.6 ± 0.1 | 7 ± 0.4 |
| 5GR-II | serotonin | 0.4 ± 0.1 | 2.2 ± 0.1 | 15 ± 2 | 25 ± 6 |
| 5GR-IV | serotonin | 0.5 ± 0.1 | 0.4 ± 0.1 | 1.5 ± 0.3 | 4.8 ± 0.8 |
| 5GR-V | serotonin | 0.7 ± 0.1 | 0.6 ± 0.1 | 1.0 ± 0.2 | 0.8 ± 0.1 |
| 5GR-VI | serotonin | 0.4 ± 0.1 | 1.3 ± 0.1 | 1.1 ± 0.3 | 9.7 ± 0.3 |
| DGR-I | dopamine | 0.7 ± 0.1 | 0.3 ± 0.1 | 0.8 ± 0.5 | 2 ± 1 |
| DGR-II | dopamine | 0.4 ± 0.1 | 0.7 ± 0.3 | 3 ± 1 | 3 ± 2 |
| DGR-III | dopamine | 0.5 ± 0.1 | 0.2 ± 0.1 | 0.6 ± 0.1 | 3 ± 1 |
| DGR-IV | dopamine | 0.4 ± 0.1 | 0.4 ± 0.2 | 1 ± 0.3 | 3 ± 2 |

^{a}Ligand concentration is 2 mM.
^{b}Percent brightness is calculated as (total fluorescence sensor, +ligand)/(total fluorescence Broccoli, +ligand).
Error is reported as the standard error of the mean for three independent experiments.

### Example 7: Discussion

RNA-based devices are progressing towards becoming a robust tool in synthetic biology, driven by a unique feature set when compared to protein-based alternatives, including the ability to regulate in cis, predictable secondary structure and a small genetic footprint. Efforts have focused on creating synthetic riboswitches, aptazymes and fluorogenic RNA sensors, but their potential has yet to be fully realized in significant part due to the limited availability of small molecule receptors that function in the context of such devices. In the work presented herein, a strategy has been designed that exploits the secondary and tertiary structural architecture of naturally evolved riboswitches and ribozymes to scaffold small molecule binding pockets raised through in vitro selection. Importantly, using no information beyond that obtained from high throughput sequencing of the final round of selection, aptamers selected to L-DOPA using this approach were coupled to a fluorogenic aptamer module to produce genetically encodable biosensors that function in the cellular context.

One key strength of the methods and compositions described herein is the use of multiple scaffolds in parallel selections to obtain a suite of aptamers. This differs significantly from traditional selections known in the art where the same subset of solutions is reproducibly generated from a simple randomized pool which significantly constrains sensor diversity and development. While the aptamers derived from different scaffolds have similar affinities for 5HTP and selectivity against L-tryptophan, they clearly have distinct characteristics with respect to their ability to communicate with a readout domain via the P1 helix, a common feature to all of the scaffolds. In biological riboswitches, the ligand is either in direct contact or induces conformational changes in the RNA that involve the P1 helix that links the aptamer to the downstream regulatory switch. Without intending to be bound by scientific theory, it is hypothesized that differences in sensor performance across different aptamers is in part due to variation in the spatial relationship between the ligand and the interdomain (P1) helix, a feature that cannot be fully controlled in the selection. However, unlike deep selections, the scaffolded selection approach presented here strongly biases selections towards a favorable ligand/P1 orientation by constraining the possible ligand position.

With a suite of aptamers, combinatorial approaches can be employed to rapidly screen for sensors without extensive aptamer characterization or device optimization as typified by the dopamine sensor development herein (FIG. 19). Development of an RNA device from aptamers derived from deep selections requires thorough characterization along with broad screening of communication modules while leaving the sensory aptamer as a fixed, unalterable node due to the lack of diversity. With the scaffolded selection methods and compositions described herein, a set of distinct aptamers can be combinatorially coupled to a set of communication modules and rapidly screened for variants with the desired activity, as demonstrated with the L-DOPA selection. In this fashion, the methods and compositions provided herein should facilitate the expedient development of RNA devices and sensors by easing a key bottleneck in their development. Notably, while in this study only the most populous clusters were focused on in each selection for characterization and/or sensor design, within each selection there are many clusters containing alternative sequences that could further enrich the initial pool of aptamers for developing downstream applications.

A second powerful advantage of the selection methods and compositions described herein is the potential for robust folding in the cellular context provided by the tertiary interaction of the three-way junction architecture. Each of these aptamers has a fold that has undergone extensive biological evolution. Further, the distal tertiary interactions that organize the three-way junction core can be highly stable. Both the L2-L3 interaction of the purine riboswitch and the tetraloop-tetraloop receptor of the cyclic di-GMP riboswitch scaffold are capable of stably forming outside the context of other RNA structure. In contrast, the long range interaction organizing the S. mansoni hammerhead ribozyme is dynamic, which is another aspect of diversity with respect to the chosen scaffolds. The presence of robust secondary and tertiary structure in the scaffold enables these elements to potentially guide the folding of all members of the initial library. In contrast, RNA misfolding during selection and or the presence of multiple MFE structures in the final aptamers is often a significant problem for traditional deep selection. Since there is no significant selection pressure for high-fidelity folding in a typical selection protocol, providing this information in the starting library can be a path towards robust folding RNAs.

While three-way junction scaffolds were chosen as the focus of this study, the diversity of natural riboswitches and ribozymes can provide further feedstock for this approach. Within the three-way junction family, there is a broad array of sequences that vary the orientation of the three helices, size of the joining regions, and the nature of the distal tertiary interaction that may provide superior scaffolds for a particular ligand or sensor. Furthermore, other folds may be predisposed to bind a target small molecule based on the nature of the cognate ligand. For example, another logical choice for a scaffold to bind 5HTP is the lysine riboswitch aptamer domain. Larger ligands may be more easily recognized by flavin mononucleotide or cobalamin riboswitch-derived scaffolds, while dinucleotides such as NADH may be readily accommodated by one of the di-cyclic nucleotide aptamers. As natural RNA aptamers have been discovered to recognize chemically diverse small molecules, exploiting their architectures towards the selection of novel aptamers has the potential to facilitate the development of powerful new tools for monitoring and responding to small molecules in the cellular environment across a broad range of applications.

### Example 8: Library Construction

For each scaffold, nucleotides within an 8 Å shell surrounding the ligand binding site or active site of the parent RNA were identified from their crystal structure (GR, PDB ID 4FE5; CDG, PDB ID 3IWN; HH, PDB ID 3ZD5). The corresponding positions were randomized in a DNA ultramer that spanned the entire aptamer domain with conserved flanking sequences for reverse transcription and amplification (Integrated DNA Technologies; sequences of all nucleic acids used in this study are provided in Table 1). ssDNA was converted into dsDNA templates for transcription using standard Taq PCR conditions in which ˜2×10⁻¹² mol DNA (corresponding to ˜10¹² individual sequences) was used in each 100 μL PCR reaction and amplified for 15 cycles with the T7 site appending and RT-PCR primers. Approximately 1×10¹⁴ sequences were transcribed in 12.5 mL transcription reaction containing 40 mM Tris-HCl, pH 8.0, 25 mM DTT, 2 mM spermidine, 0.01% Triton X-100, 4 mM each rNTP pH 8.0, 0.08 units inorganic phosphatase (Sigma-Aldrich, lyophilized powder), and 0.25 mg/mL T7 RNA polymerase and incubated at 37° C. for 4 hours. Transcription samples were then precipitated in 75% ethanol at −20° C., pelleted, and reconstituted in a solution of 300 μL formamide, 3 mL 8 M Urea, and 300 μL 0.5 M EDTA pH 8.0. Full length RNA was purified with a denaturing 8%, 29:1 acrylamide:bisacrylamide gel. Product RNA was excised from the gel after visualizing by UV shadowing and eluted in 0.3 M NaOAc pH 5.0 before exchange and storage in 0.5×TE.

### Example 9: Synthesis of 5HTP Affinity Column Matrix

For the derivatized columns, 3 mL bed volume of EAH Sepharose 4B (GE Healthcare) was dehydrated with dimethylformamide (DMF). 10 μmoles of Fmoc-5-hydroxy-L-tryptophan and 10 μmoles of benzotriazol-1-yl-oxytripyrrolidinophosphonium hexafluorophosphate (PyBOP) were dissolved in 1 mL of DMF and added to the dehydrated column with 20 μmoles N,N-diisopropylethylamine (DIPEA) and incubated with agitation for 2 hours at room temperature. The column matrix was then drained and washed extensively with DMF. Unreacted sepharose amines were acetylated by adding 1 mmole of acetic anhydride and 1 mmole DIPEA in approximately 1 mL DMF and mixed at room temperature for 1 hour. The column was drained of the acetylating mixture and washed with DMF prior to Fmoc deprotection using 20% v/v piperidine/DMF. Amino acid concentration on the column was determined by measuring the concentration of Fmoc in the deprotection fractions (A_{301 nm}=8000 M⁻¹ cm⁻¹). This method generated approximately 0.5-1 mM deprotected amino acid per mL resin. For counter selection, EAH sepharose was prepared exactly the same except omitting the ligand coupling step resulting in acetylated sepharose.

### Example 10: In Vitro Selection

For the GR scaffold selection using the SuperScript III reverse transcriptase (GR/SSIII), 350 μL acetylated sepharose was equilibrated in selection buffer (10 mM Na-HEPES, pH 7.0, 250 mM NaCl, 50 mM KCl, 10 mM MgCl₂, 0.1 mg/mL tRNA) and 1 nmol of library RNA in 350 μL of selection buffer was incubated at room temperature for 30 minutes with agitation.

The applied solution was removed and the column matrix washed once with 350 μL of selection buffer. The pooled flow through and wash (750 μL total) was added to pre-equilibrated 5HTP-derivatized sepharose 4B column and incubated for 45 minutes. The column was then drained and washed three times with selection buffer before elution with 10 mM 5HTP in selection buffer (two 1 hour incubations in 350 μL; total 700 μL eluted volume). The eluted fractions were then concentrated to 50 μL in a 0.5 mL Ultracel 10 kD MWCO filter (Millipore) and ethanol precipitated in 0.3 M sodium acetate (pH 5.0), 5 μg glycogen, and brought to a final concentration of 75% ethanol before storage at −70° C. for 30 minutes. Details of the conditions of each cycle are provided in Table 2.

To convert the competitively eluted RNA into a new population of RNA, the elution fractions were ethanol precipitated, pelleted at 13000×g at 4° C., decanted, and dried under vacuum. The dried pellet was reconstituted with 0.7 mM each dNTP, 7 μM RT-PCR primer, and brought to a total volume of 14 μL before heating to 65° C. for 5 minutes and incubation on ice for 10 minutes. The solution was then brought up to 1× SuperScript III first-strand buffer (5×: 250 mM Tris-HCl, pH 8.3, 375 mM KCL, 15 mM MgCl₂) with 5 mM DTT and 200 units SuperScript III (Life Technologies) in a total volume of 20 μL before a 15 minute extension at 54° C. The entire 20 μL reverse transcription solution was PCR amplified in a total volume of 500 μL using standard Taq DNA polymerase conditions. The amplified pool was then transcribed by adding 100 μL of the PCR reaction to a 1 mL transcription reaction containing 40 mM Tris-HCl, pH 8.0, 25 mM DTT, 2 mM spermidine, 0.01% Triton X-100, 4 mM each rNTP pH 8.0, 0.08 units inorganic phosphatase, and 0.25 mg/mL T7 RNA polymerase and incubated at 37° C. for 2 hours. A 100 μL transcription reaction for ³²P labeled RNA was performed under similar condition with the exception that the rNTPs were lowered to 2 mM for UTP, CTP, and GTP while ATP was reduced to 200 μM and ˜100 μCi ³²P-ATP. Transcription samples were gel purified as described above with the gel loading conditions scaled accordingly.

Selections using the GsI reverse transcriptase were performed as described above with the following changes. The buffer for selection contained a reduced magnesium concentration and more physiologically relevant monovalent cations: 25 mM Na-HEPES, pH 7.0, 150 mM KCl, 50 mM NaCl, 3 mM MgCl₂). GsI-IIC-MRF reverse transcriptase was used in place of SuperScript III. GsI-IIC MRF reverse transcriptase was expressed in E. coli and purified as described (Mohr et al., RNA, 2013, 19, 958-970). The precipitated RNA pellet was brought up in 1.25 mM dNTPs and 20 μM RT-PCR primer prior to denaturation at 65° C., annealing at 4° C., and equilibration at 60° C. The solution was then brought up to 1× GsI-IIC-MRF buffer conditions (10 mM NaCl, 1 mM MgCl₂, 20 mM TrisCl, pH 7.5, 1 mM DTT) in 20 μL total volume and sufficient enzyme was added for extension at 60° C. PCR was as performed as described above.

### Example 11: High-Throughput Sequencing and Bioinformatic Analysis

Standard PCR was conducted to append the Illumina hybridization sequences necessary for annealing to the flow cell. Each library was amplified with the forward sequencing primer and a unique reverse primer containing a distinguishing 12 nucleotide barcode (sequences are given in Table 1). The samples were sequenced using a v3 reagents kit for 150 cycles on a MiSeq (Illumina) with custom read and indexing primers.

The resulting sequences were demultiplexed, trimmed, and quality filtered using scripts from QIIME (Caporaso et al., Nat. Methods, 2010, 7, 335-336). All sequence information outside of the P1 stem was trimmed and only sequences containing a Phred score≥20 for each nucleotide were used in the analysis. The resulting fasta format files for each library were then subjected to clustering by USEARCH (Edgar, Bioinformatics, 26, 2460-2461), which generated seed sequences that were clustered at 90% identity; any clusters containing a single sequence were discarded. The top ten populous clusters were then mapped back to their original sequence file, and 250 individual sequences were randomly taken as a representative sample of each cluster for further analysis. Sequences in each cluster were aligned using MUSCLE (Edgar, NAR, 2004, 32, 1792-1797) and the resultant alignment analyzed using CMfinder (Yao et al., Bioinformatics, 2006, 22, 445-452). R2R (Weinberg & Breaker, BMC Bioinformatics, 2011, 12, 3) was run at its default settings to generate figures of the sequence conservation mapped onto the minimum free energy (MFE) secondary structure.

### Example 12: NMIA Chemical Probing

RNA was prepared as described previously (Edwards et al., Methods Mol. Biol., 2009, 535, 135-163). Structure cassettes flanking the 5′ and 3′ ends of the RNA were added to facilitate reverse transcription and NMIA modification was performed using the established protocols (Wilkinson et al., Nat. Protoc., 2006, 1, 1610-1616) at 25° C. RNA was probed at 100 nM in 100 mM Na-HEPES, pH 8.0, 100 mM NaCl, and 6 mM MgCl₂. Ligand concentration was 500 μM where indicated. Gel images were analyzed by SAFA (Das et al., RNA, 2005, 11, 344-354) and ImageJ (NIH).

### Example 13: Isothermal Titration Calorimetry (ITC)

All RNAs tested were exchanged into the SSIII selection buffer (10 mM Na-HEPES, pH 7.0, 250 mM NaCl; 50 mM KCl; 10 mM MgCl₂) and washed three times in 10 kD MWCO filter (EMD Millipore). The ligand was brought up from a dry solid directly into the binding buffer and concentration established on a NanoDrop 2000 (Thermo Scientific) using an extinction coefficient at 275 nm of 8000 mol⁻¹ cm⁻¹ for the 5-hydroxyindole moiety. The RNA was diluted to between 50-100 μM and the ligand was titrated at roughly 10 times the concentration of RNA. Titrations were performed at 25° C. using an MicroCal iTC200 microcalorimeter (GE Healthcare) using established protocols (Gilbert and Batey, Methods Mol. Biol., 2009, 540, 97-114). Data was analyzed and fitting was performed with the Origin 5.0 software suite (Origin Laboratories).

### Example 14: Structure Determination of the 5HTP-II/5HTP Complex

RNA for crystallization was prepared as previously described (Edwards et al., Methods Mol. Biol., 2009, 535, 135-163). The RNA was concentrated in an Amicon Ultra 15 10 k MWCO filter (EMD Millipore, Inc.) and exchanged into 0.5× T.E. buffer. Diffraction quality crystals were obtained by mixing 2 μL RNA:ligand complex (1:1) and 3.5 μL mother liquor (8-14% 2-methyl-2,4-pentanediol, 40 mM sodium cacodylate pH 5.5, 4 mM MgCl₂, 12 mM NaCl, 80 mM KCl, and 4-9 mM cobalt hexamine), micro-seeding, and incubation at 22° C. for 1-3 days. The crystals needed no further cryoprotection and were flash frozen in liquid nitrogen before data collection. Data was collected with a Rigaku R-Axis IV image plate system using CuKα radiation (1.5418 Å) at 100 K, and was indexed and scaled using D*TREK (Pflugrath, Acta Crystallogr. D Biol. Crystallogr., 1999, 55, 1718-1725). Data on a heavy atom derivative made by replacing cobalt hexamine with 1-11 mM iridium hexamine was also collected on the home x-ray source. Phases were determined using the single isomorphous replacement with anomalous scattering (SIRAS) method. AutoSol (Adams et al., Acta Crystallogr. D Biol. Crystallogr., 2010, 66, 213-221) was used to find 12 iridium atoms that were then used to calculate phases. The resulting experimental density map displayed unambiguous features of the RNA backbone and helices and was used for building the model.

The initial model was iteratively built without the ligand in Coot (Emsley & Cowtan, Acta Crystallogr. D Biol. Crystallogr., 2004, 60, 2126-2132) between rounds of refinement in PHENIX (Adams et al., Acta Crystallogr. D Biol. Crystallogr., 2010, 66, 213-221). The RNA model was brought through several rounds of refinement and simulated annealing before 5HTP was built into the model. At this point of building, there was clear ligand density in the binding pocket that allowed for the confident placement and orientation of the ligand. The placement of the ligand and bases was validated by a composite omit map (FIG. 10B). Water placement was automated in final rounds of refinement after ligand placement based on peak size in the Fₒ-F_{c} difference map. The resultant model had good geometry as judged using MolProbity (Chen et al., 2010, Acta Crystallogr. D Biol. Crystallogr., 2010, 66, 213-221) and final model statistics (R_{work} and R_{free} are 21.9% and 26.2%, respectively). All crystallographic data and model statistics are given in Table 4.

### Example 15: In Vitro Broccoli Sensor Assays

RNA was prepared as described above, with additional 0.5× T.E. buffer washes in a 10 k MWCO Amicon Ultra (Millipore) to minimize carry-over of metal ions. All RNA sensors were assayed at concentrations of 0.5 μM RNA and 10 μM (Z)-4-(3,5-difluoro-4-hydrozybenzylidene)- 1,2-dimethyl-1H-imidazol-5(4H)-one (DFHBI) in a buffer containing 80 mM Tris-HCl, pH 7.4, 150 mM KCl, and 50 mM NaCl. The buffer, ligand, magnesium (concentrations given in Table 6), and DFHBI were mixed prior to the addition of RNA and all reactions allowed to incubate for 30 minutes at room temperature. DFHBI fluorescence was measured by placing 200 μL reaction volume in a Greiner 96-well flat bottom black fluorescence plate (Thermo Scientific) and reading in a Tecan Infinite M200 PRO plate reader. Samples were excited at 460 nm and fluorescence emission measured as the average signal between 506 and 510 nm. The concentration of ligand to elicit a half maximal fluorescence response was determined by fitting the observed fluorescence as a function of ligand concentration to a two state model.

Engineered sensors were synthesized as G-blocks (sequences of sensors given in FIG. 23; Integrated DNA Technologies) and cloned between the XbaI and BlpI sites in pET30b using standard molecular cloning techniques. All resultant plasmids were sequence verified. For T7 RNA polymerase transcription reactions, a DNA template was generated by PCR using 1 μM outer primers (5′: GGCCGTAATACGACTCACTATAGGAGCCCGGATAGCTCAGTCGGTAGAGCAG, 3′: TGGCGCCCGAACAGGGACTTGAACCCTGGA) using a standard PCR reaction. Templates were directly added to an in vitro transcription reaction (see above) and RNA synthesis was allowed to proceed for 2 hours at 37° C. RNA from the above transcription reaction was directly used in assays without further purification.

The activity of each sensor was monitored in a 100 μL reaction containing 50 μL of in vitro transcription reaction, 10 μL of 10× Survey Buffer (1×: 50 mM K-HEPES, pH 7.5, 10 mM MgCl₂, 150 mM KCl, 50 mM NaCl), 30 μM DFHBI-1T and 2 mM ligand (for plus ligand reactions). Reactions were incubated at room temperature for 30 minutes and DFHBI fluorescence was measured by placing 90 μL reaction volume in a Greiner 96-well flat bottom black fluorescence plate (Thermo Scientific) and reading in a Tecan Infinite M200 PRO plate reader. Samples were excited at 460 nm and fluorescence emission measured as the average signal between 506 and 510 nm. For all experiments, a positive control of a tRNA-scaffolded Broccoli aptamer was performed in the presence and absence of ligand, which was also used as a reference for relative brightness. Fold-induction was calculated by dividing the fluorescence values for the DFHBI-1T plus ligand reaction by the fluorescence value for the DFHBI-1T condition alone. All experiments were performed in triplicate and quantified data reported with the standard error of the mean (s.e.m.)

### Example 16: In Vitro Broccoli Sensor Assays

E. coli One Shot® BL21 Star (DE3) cells (Thermo Fisher) were transformed with a pET30b-derived plasmid containing a sensor under inducible control, plated onto LB agar supplemented with 50 μg/mL kanamycin and incubated at 37° C. for approximately 16 hours.

Individual colonies were picked and grown overnight (approximately 16 hours) in 5 mL of LB supplemented 50 μg/mL kanamycin to allow the culture to reach saturation. For screening experiments, 5 μL of the saturated overnight culture was added to 5 mL of LB supplemented with 50 μg/mL kanamycin and grown to mid-log phase (OD600 approximately 0.4-0.6) at 37° C. To induce expression of the Broccoli aptamer alone or the Broccoli/riboswitch aptamer fusion constructs, IPTG was added to a final concentration of 1 mM in each culture, which were then grown for an additional 2 hours at 37° C. Cells were then pelleted by centrifugation and washed once with 5 mL of 1× M9 salts supplemented with MgSO₄ at a final concentration of 5 mM and kanamycin at a final concentration of 50 μg/mL. After washing, cells were pelleted by centrifugation, resuspended in 250 μL of the above M9 medium and split into two 100 μl aliquots. In half of the aliquots, DFHBI-1T was added to a final concentration of 50 μM in a final volume of 110 μL. In the other half of the aliquots, DFHBI-1T was added to a final concentration of 50 μM and the ligand (5HTP, 5HP or dopamine) was added to a final concentration of 1 mM in a final volume of 110 μL. Cells were then incubated at 37° C. for 30 minutes to allow for uptake of each compound. Following the 30 minute incubation, 100 μL of each aliquot was pipetted into a Greiner 96-well black microplate and chilled on ice for 30 minutes. For fluorescence measurements, DFHBI-1T was monitored at an excitation wavelength of 472 nm and a 520 nm emission wavelength. Quantified data represent the average fluorescence values±standard error of the mean (s.e.m.) from three biological replicates, which were background corrected using a pET30b empty vector control. Fold-induction was calculated by dividing the average fluorescence values of cells exposed to ligand by the average fluorescence of cells without ligand.

### Example 17: Intracellular Fluorescence Imaging of 5HTP

DNA and cultures were prepared as described (Paige et al., Science, 2012, 335, 1194). Briefly, the tRNA/Broccoli fusion sequence was cloned into pET30b between the XbaI and BlpI sites downstream of an inducible T7 promoter. The sequence-verified plasmid was transformed into BL21 (DE3) STAR cells (Invitrogen) and single colonies were grown up overnight in Luria Broth (LB) supplemented with 50 μg/mL kanamycin. The overnight culture was used to inoculate fresh LB/kanamycin medium at a 1:1000 dilution and the culture grown at 37° C. to an OD₆₀₀=0.4-0.6 before induction with 1 mM IPTG and growth at 37° C. for 2-4 hours. 200 μL of the resultant culture was centrifuged, decanted, and resuspended in 2 mL of M9 minimal salts medium supplemented with 50 μg/mL kanamycin, 5 mM MgSO₄, and 1 mM IPTG. 200 μL of the resuspended culture was transferred to 96-well poly-D-lysine coated glass bottom plates (MatTek) and incubated at 37° C. for one hour. The media was then removed and the wells washed with M9/kanamycin/1 mM IPTG medium before adding 200 μL of M9 media, 1 mM IPTG, and 400 μM DFHBI-1T (Lucerna). The live fluorescence images were taken with an Andor iXon3 897 EMCCD using a 60× oil objective, an excitation filter 472/30, dichroic mirror 490 (long pass) and emission filter 520/40 on a Nikon Ti-E microscope and analyzed with FIJI (Schindelin et al., Nat. Methods, 2012, 9, 676-682).

### Example 18: Single Turnover In Vitro Transcription Assays

dsDNA templates were transcribed as previously described (Trausch et al., Structure, 2011, 19, 1413-1423). In brief, 50 ng of DNA template were incubated at 37° C. for 10 minutes in 12.5 μL of 2× transcription buffer (140 mM Tris-HCl, pH 8.0, 140 mM NaCl, 0.2 mM EDTA, 28 mM β-mercaptoethanol and 70 mg/mL BSA), 2.5 μL 50 mM MgCl₂, 100-200 μCi of ³²P-ATP, and 0.25 units of E. coli RNA polymerase σ70 holoenzyme (Epicentre Biotechnologies) per reaction were brought to 23 μL. The equilibrated reactions were then initiated with the addition of 7.5 μL reaction buffer (165 μM each rNTP, 0.2 mg/mL heparin, and the desired ligand concentration) and incubated for 15 minutes at 37° C. before quenching with 8 M urea. The reactions were then separated on an 8% denaturing PAGE, dried, and exposed on a phosphor imager screen. Quantitation of the gels then carried out in ImageJ (NIH) and the data fit to a two-state model.

### ACCESSION CODES

Coordinates and structure factors have been deposited in the RSCB Protein Data Bank under the accession code 4ZAQ.

### INCORPORATION BY REFERENCE

The contents of all references (including literature references, issued patents, published patent applications, and co-pending patent applications) cited throughout this application are hereby expressly incorporated herein by reference in their entireties. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments provided herein. Such equivalents are intended with be encompassed by the following claims.

What is claimed is:

1. A library of oligonucleotides comprising a plurality of non-identical oligonucleotides, wherein individual oligonucleotides comprise:
a) a first sequence comprising a helix domain;
b) a second sequence comprising a first hairpin domain; and
c) a third sequence comprising a second hairpin domain;
wherein the helix domain, first hairpin domain and second hairpin domain form an oligonucleotide junction containing a ligand-binding domain, and
wherein the library comprises a plurality of non-identical ligand-binding domains.

2. The library of oligonucleotides of claim 1, wherein each helix domain independently is a fully complementary helix optionally comprising one or more destabilizing nucleotides selected from the group consisting of a mismatched base pair, a G⋅U wobble base pair and a bulge.

3. The library of oligonucleotides of claim 1, wherein each helix domain is a fully complementary helix.

4. The library of oligonucleotides of claim 1, wherein each first hairpin domain independently comprises one or more destabilizing nucleotides selected from the group consisting of a mismatched base pair, a G⋅U wobble base pair and a bulge.

5. The library of oligonucleotides of claim 1, wherein each second hairpin domain independently comprises one or more destabilizing nucleotides selected from the group consisting of a mismatched base pair, a G⋅U wobble base pair and a bulge.

6. The library of oligonucleotides of claim 1, wherein the helix domain is at least 4 to 10 base-pairs in length.

7. The library of oligonucleotides of claim 1, wherein the helix domain is at least 10 base-pairs in length.

8. The library of oligonucleotides of claim 1, wherein the oligonucleotides are oligoribonucleotides.

9. The library of oligonucleotides of claim 1, wherein the oligonucleotides individually comprise a sequence having a series of linked sequences according to Formula I:
P1-J1/2-P2-L2-P2′-J2/3-P3-L3-P3′-J3/1-P1′ (I)
wherein
represents a bond;
P1 and P1′ form the helix;
P2, L2 and P2′ form the first hairpin;
P3, L3 and P3′ form the second hairpin; and
J1/2, J2/3 and J3/1 together form the oligonucleotide junction.

10. The library of oligonucleotides of claim 9, wherein J2/3 comprises a T-loop motif.

11. The library of oligonucleotides of claim 10, wherein the T-loop motif comprises the sequence UUGAA.

12. The library of oligonucleotides of claim 11, wherein the guanosine of the T-loop forms a Watson-Crick base-pair with a cytidine in J3/1.

13. The library of oligonucleotides of claim 1, wherein the helix domain has a first end and a second end, and the first end is proximal to the oligonucleotide junction, and the second end is linked to an oligonucleotide-based readout module.

14. The library of oligonucleotides of claim 13, wherein the oligonucleotide-based readout module is a fluorogenic or switch-based readout module.

15. The library of oligonucleotides of claim 14, wherein the fluorogenic module is a Broccoli fluorophore binding aptamer.

16. The library of oligonucleotides of claim 14, wherein the switch-based module is a pbuE switch.

17. The library of oligonucleotides of claim 13, wherein the oligonucleotide-based readout module is an oligoribonucleotide-based readout module.

18. The library of oligonucleotides of claim 1, wherein individual oligonucleotides have sequence correspondence to a Bacillus subtilis xpt-pbuX guanine riboswitch sequence, comprising about 23 variable nucleotide residues within the oligonucleotide junction.

19. The library of oligonucleotides of claim 1, wherein individual oligonucleotides have sequence correspondence to a Vibrio cholera Vc2 cyclic di-GMP riboswitch sequence, comprising about 21 variable nucleotide residues within the oligonucleotide junction.

20. The library of oligonucleotides of claim 1, wherein individual oligonucleotides have sequence correspondence to a Schistosoma mansoni hammerhead ribozyme sequence, comprising about 21 variable nucleotide residues within the oligonucleotide junction.

21. The library of oligonucleotides of claim 1, wherein the oligonucleotide junction is an N-way junction, wherein N is two, three, four or five.

22. The library of oligonucleotides of claim 1, wherein the oligonucleotide junction is an N-way junction, wherein N is two.

23. The library of oligonucleotides of claim 1, wherein the oligonucleotide junction is an N-way junction, wherein N is three.

24. The library of oligonucleotides of claim 1, wherein the oligonucleotide junction is an N-way junction, wherein N is four.

25. The library of oligonucleotides of claim 1, wherein the oligonucleotide junction is an N-way junction, wherein N is five.

26. The library of oligonucleotides of claim 1, wherein the library comprises from about 4²¹ to about 4²³ non-identical members.

27. A library of oligonucleotides comprising a plurality of non-identical oligonucleotides, wherein individual oligonucleotides comprise:
a) a first sequence comprising a helix domain;
b) a second sequence comprising a first hairpin domain; and
c) a third sequence comprising a second hairpin domain;
wherein the helix domain, the first hairpin domain and the second hairpin domain form an oligonucleotide junction containing a pre-selected ligand-binding domain, and
wherein the library comprises a plurality of non-identical ligand-binding domains.

28. The library of oligonucleotides of claim 27, wherein each helix domain independently is a fully complementary helix optionally comprising one or more destabilizing nucleotides selected from the group consisting of a mismatched base pair, a G⋅U wobble base pair and a bulge.

29. The library of oligonucleotides of claim 27, wherein each helix domain is a fully complementary helix.

30. The library of oligonucleotides of claim 27, wherein each first hairpin domain independently comprises one or more destabilizing nucleotides selected from the group consisting of a mismatched base pair, a G⋅U wobble base pair and a bulge.

31. The library of oligonucleotides of claim 27, wherein each second hairpin domain independently comprises one or more destabilizing nucleotides selected from the group consisting of a mismatched base pair, a G⋅U wobble base pair and a bulge.

32. The library of oligonucleotides of claim 27, wherein the helix domain is at least 4 to 10 base-pairs in length.

33. The library of oligonucleotides of claim 27, wherein the helix domain is at least 10 base-pairs in length.

34. The library of oligonucleotides of claim 27, wherein the oligonucleotides are oligoribonucleotides.

35. The library of oligonucleotides of claim 27, wherein individual oligonucleotides comprise a sequence having a series of linked sequences according to Formula I:
P1-J1/2-P2-L2-P2′-J2/3-P3-L3-P3′-J3/1-P1′ (I)
wherein
represents a bond;
P1 and P1′ form the helix;
P2, L2 and P2′ form the first hairpin;
P3, L3 and P3′ form the second hairpin; and
J1/2, J2/3 and J3/1 together form the oligonucleotide junction.

36. The library of oligonucleotides of claim 35, wherein J2/3 comprises a T-loop motif.

37. The library of oligonucleotides of claim 36, wherein the T-loop motif comprises the sequence UUGAA.

38. The library of oligonucleotides of claim 37, wherein the guanosine of the T-loop forms a Watson-Crick base-pair with a cytidine in J3/1.

39. The library of oligonucleotides of claim 27, wherein the helix domain has a first end and a second end, and the first end is proximal to the oligonucleotide junction, and the second end of is linked to an oligonucleotide-based readout module.

40. The library of oligonucleotides of claim 39, wherein the oligonucleotide-based readout module is a fluorogenic or switch-based readout module.

41. The library of oligonucleotides of claim 40, wherein the fluorogenic module is a Broccoli fluorophore binding aptamer.

42. The library of oligonucleotides of claim 40, wherein the switch-based module is a pbuE switch.

43. The library of oligonucleotides of claim 39, wherein the oligonucleotide-based readout module is an oligoribonucleotide-based readout module.

44. The library of oligonucleotides of claim 27, wherein individual oligonucleotides comprise sequences having sequence correspondence to a Bacillus subtilis xpt-pbuX guanine riboswitch sequence, comprising about 23 variable nucleotide residues within the oligonucleotide junction.

45. The library of oligonucleotides of claim 27, wherein individual oligonucleotides comprise sequences having sequence correspondence to a Vibrio cholera Vc2 cyclic di-GMP riboswitch sequence, comprising about 21 variable nucleotide residues within the oligonucleotide junction.

46. The library of oligonucleotides of claim 27, wherein individual oligonucleotides comprise sequences having sequence correspondence to a Schistosoma mansoni hammerhead ribozyme sequence, comprising about 21 variable nucleotide residues within the oligonucleotide junction.

47. The library of oligonucleotides of claim 27, wherein the oligonucleotide junction is an N-way junction, wherein N is two, three, four or five.

48. The library of oligonucleotides of claim 27, wherein the oligonucleotide junction is an N-way junction, wherein N is two.

49. The library of oligonucleotides of claim 27, wherein the oligonucleotide junction is an N-way junction, wherein N is three.

50. The library of oligonucleotides of claim 27, wherein the oligonucleotide junction is an N-way junction, wherein N is four.

51. The library of oligonucleotides of claim 27, wherein the oligonucleotide junction is an N-way junction, wherein N is five.

52. The library of oligonucleotides of claim 27, wherein the preselected ligand-binding site comprises a binding site for a compound selected from the group consisting of an amino acid, a peptide, a nucleobase, a nucleoside, a nucleotide, a metal ion, a neurotransmitter, a hormone, an active pharmaceutical ingredient, and derivatives thereof.

53. The library of oligonucleotides of claim 52, wherein the preselected ligand-binding site comprises a binding site for a ligand selected from the group consisting of an amino acid, a nucleobase, a nucleoside, a nucleotide, a neurotransmitter, a hormone, and derivatives thereof.

54. The library of oligonucleotides of claim 53, wherein the preselected ligand-binding site comprises a binding site for a ligand selected from the group consisting a nucleotide, a neurotransmitter, a hormone, and derivatives thereof.

55. The library of oligonucleotides of claim 27, wherein the preselected ligand-binding site comprises a binding site for at least one ligand selected from the group consisting of 5-hydroxy-L-tryptophan, L-tryptophan, serotonin, and 5-hydroxy-L-tryptophan-methylamide.

56. The library of oligonucleotides of claim 55, wherein the ligand is at least one of 5-hydroxy-L-tryptophan or serotonin.

57. A method of selecting a plurality of non-identical ligand-binding oligonucleotides, comprising the steps of:
1) contacting a library of oligonucleotides comprising a plurality of oligonucleotides with a ligand under conditions suitable for ligand binding, wherein individual oligonucleotides comprise:
a) a first sequence comprising a helix domain;
b) a second sequence comprising a first hairpin domain; and
c) a third sequence comprising a second hairpin domain;
wherein the helix domain, first hairpin domain and second hairpin domain form an oligonucleotide junction; and
2) partitioning the library of oligonucleotides in a spatially addressable such that the plurality of non-identical ligand-binding oligonucleotides is selected, wherein the oligonucleotides having the oligonucleotide junction further comprise a ligand-binding domain, and wherein the ligand-binding domains of the library of oligonucleotides comprise variable nucleotide residues, is selected.

58. The method of claim 57, wherein the method further comprises a step 1a) between step 1) and step 2), step 1a) comprising competitively partitioning the library of oligonucleotides with a solution of free ligand.


SEQUENCE LISTING (118 sequences)

SEQ ID NO: 1 - DNA, 149 bp, Artificial Sequence
  GR/SSIII library template
  misc_feature (56)..(62): n is a, c, g, or t
  misc_feature (82)..(91): n is a, c, g, or t
  misc_feature (109)..(114): n is a, c, g, or t
gcgcgcgaattctaatacgactcactataggacttcggtccaagctaatgcactcnnnnnnncgcgtggatatggcacgcannnnnnnnnngggcaccgtaaatgtccnnnnnngggtgcattagcaaaatcgggcttcggtccggttc

SEQ ID NO: 2 - DNA, 147 bp, Artificial Sequence
  GR/GsI library template
  misc_feature (55)..(61): n is a, c, g, or t
  misc_feature (81)..(90): n is a, c, g, or t
  misc_feature (108)..(113): n is a, c, g, or t
gcgcgcgaattctaatacgactcactataggacttcggtccttggataggactcnnnnnnncgcgtggatatggcacgcannnnnnnnnngggcaccgtaaatgtccnnnnnngggtcctatccccaatcgggcttcggtccggttc

SEQ ID NO: 3 - DNA, 164 bp, Artificial Sequence
  CDG/GsI library template
  misc_feature (54)..(62): n is a, c, g, or t
  misc_feature (87)..(91): n is a, c, g, or t
  misc_feature (125)..(131): n is a, c, g, or t
gcgcgcgaattctaatacgactcactataggacttcggtccttggataggacannnnnnnnncaaaccattcgaaagagtgggacgnnnnncctccggcctaaaccgaaaggtaggtagcggggnnnnnnntgtcctatccccaatcgggcttcggtccggttc

SEQ ID NO: 4 - DNA, 143 bp, Artificial Sequence
  HH/GsI library template
  misc_feature (54)..(57): n is a, c, g, or t
  misc_feature (84)..(93): n is a, c, g, or t
  misc_feature (104)..(110): n is a, c, g, or t
gcgcgcgaattctaatacgactcactataggacttcggtccttggataggagcnnnntggtatccaatgaaaatgtactaccannnnnnnnnncccaaataggnnnnnnngctcctatccccaatcgggcttcggtccggttc

SEQ ID NO: 5 - DNA, 55 bp, Artificial Sequence
  T7 site appending primer
gcgcgcgaattctaatacgactcactataggacttcggtccaagctaatgcactc

SEQ ID NO: 6 - DNA, 18 bp, Artificial Sequence
  RT-PCR primer
gaaccggaccgaagcccg

SEQ ID NO: 7 - DNA, 66 bp, Artificial Sequence
  HTS reverse sequencing primer, GR/SSIII
caagcagaagacggcatacgagatgtcgtgtagcctagtcagtcagccgaaccggaccgaagcccg

SEQ ID NO: 8 - DNA, 66 bp, Artificial Sequence
  HTS reverse sequencing primer, GR/GsI
caagcagaagacggcatacgagattggtcaacgataagtcagtcagccgaaccggaccgaagcccg

SEQ ID NO: 9 - DNA, 66 bp, Artificial Sequence
  HTS reverse sequencing primer, CDG/GsI
caagcagaagacggcatacgagatatcaccaggtgtagtcagtcagccgaaccggaccgaagcccg

SEQ ID NO: 10 - DNA, 66 bp, Artificial Sequence
  HTS reverse sequencing primer, HH/GsI
caagcagaagacggcatacgagatgctgtacggattagtcagtcagccgaaccggaccgaagcccg

SEQ ID NO: 11 - DNA, 71 bp, Artificial Sequence
  Forward sequencing primer
aatgatacggcgaccaccgagatctacactatggtaattgtgcgcgcgaattctaatacgactcactatag

SEQ ID NO: 12 - DNA, 30 bp, Artificial Sequence
  RT primer
agtcagtcagccgaaccggaccgaagcccg

SEQ ID NO: 13 - DNA, 30 bp, Artificial Sequence
  Indexing primer
cgggcttcggtccggttcggctgactgact

SEQ ID NO: 14 - DNA, 74 bp, Artificial Sequence
  5HTP-II
ggacactctgatgatcgcgtggatatggcacgcattgaattgttggacaccgtaaatgtcctaacacgtgtcca

SEQ ID NO: 15 - DNA, 84 bp, Artificial Sequence
  5HTP-I aptamer
ggagctaatgcactcttaacgccgcgtggatatgcacgcaaccgtgaatcgggcaccgtaaattccgtaagtgggtgcattagc

SEQ ID NO: 16 - DNA, 73 bp, Artificial Sequence
  5HTP-II aptamer
ggcactctgatgatcgcgtggatatggcacgcattgaattgttggacaccgtaaatgtcctaacacgggtgcc

SEQ ID NO: 17 - DNA, 81 bp, Artificial Sequence
  5HTP-III aptamer
ggagctaatgcactcccattttccgtggatatggcacgctaccgatgttgggaccgtaatgtccattacgggtgcattagc

SEQ ID NO: 18 - DNA, 81 bp, Artificial Sequence
  5HTP-IV aptamer
ggataggactctctggttcgcgtagatatggcacgcaattgaagaatgggcaccgtaaatgtctgtagacgggtcctatcc

SEQ ID NO: 19 - DNA, 81 bp, Artificial Sequence
  5HTP-V aptamer
ggataggactcattcggccgcgtggatatggcacgcaggagatgtgtggacaccgtaaatgtccgtaggcgggtcctatcc

SEQ ID NO: 20 - DNA, 81 bp, Artificial Sequence
  5HTP-VI aptamer
ggataggactcaacatctcgcgtggatatggcacgcagacttccagtgggcaccgtaaatgtccgtagacgggtcctatcc

SEQ ID NO: 21 - DNA, 98 bp, Artificial Sequence
  5HTP-VII aptamer
ggataggacatgtaatctccaaaccattcgaaagagtgggacgctagacctccggcctaaaccgaaaggtaggtagcggggctaggtatgtcctatcc

SEQ ID NO: 22 - DNA, 77 bp, Artificial Sequence
  5HTP-VIII aptamer
ggataggagctgtttggtatccaatgaaaatgtactaccaacttgaatctcccaaataggctaggtagctcctatcc

SEQ ID NO: 23 - DNA, 140 bp, Artificial Sequence
  5HTP-I, SHAPE
ggacttcggtccaagctaatgcactcttaacgccgcgtggatatgcacgcaaccgtgaatcgggcaccgtaaattccgtaagtgggtgcattagcaatcgatccggttcgccggatccaaatcgggcttcggtccggttc

SEQ ID NO: 24 - DNA, 142 bp, Artificial Sequence
  5HTP-II, SHAPE
ggacttcggtccaagctaatgcactctgatgatcgcgtggatatggcacgcattgaattgttggacaccgtaaatgtcctaacacgggtgcattagcaatcgatccggttcgccggatccaaatcgggcttcggtccggttc

SEQ ID NO: 25 - DNA, 139 bp, Artificial Sequence
  5HTP-III, SHAPE
ggacttcggtccaagctaatgcactcccattttccgtggatatggcacgctaccgatgttgggaccgtaatgtccattacgggtgcattagcaaaatcgatccggttcgccggatccaaatcgggcttcggtccggttc

SEQ ID NO: 26 - DNA, 116 bp, Artificial Sequence
  5HTP-IV, SHAPE
ggacttcggtccttggataggactctctggttcgcgtagatatggcacgcaattgaagaatgggcaccgtaaatgtctgtagacgggtcctatccaatcgggcttcggtccggttc

SEQ ID NO: 27 - DNA, 116 bp, Artificial Sequence
  5HTP-V, SHAPE
ggacttcggtccttggataggactcattcggccgcgtggatatggcacgcaggagatgtgtggacaccgtaaatgtccgtaggcgggtcctatccaatcgggcttcggtccggttc

SEQ ID NO: 28 - DNA, 116 bp, Artificial Sequence
  5HTP-VI, SHAPE
ggacttcggtccttggataggactcaacatctcgcgtggatatggcacgcagacttccagtgggcaccgtaaatgtccgtagacgggtcctatccaatcgggcttcggtccggttc

SEQ ID NO: 29 - DNA, 133 bp, Artificial Sequence
  5HTP-VII, SHAPE
ggacttcggtccttggataggacatgtaatctccaaaccattcgaaagagtgggacgctagacctccggcctaaaccgaaaggtaggtagcggggctaggtatgtcctatccaatcgggcttcggtccggttc

SEQ ID NO: 30 - DNA, 112 bp, Artificial Sequence
  5HTP-VIII, SHAPE
ggacttcggtccttggataggagctgtttggtatccaatgaaaatgtactaccaacttgaatctcccaaataggctaggtagctcctatccaatcgggcttcggtccggttc

SEQ ID NO: 31 - DNA, 108 bp, Artificial Sequence
  5HTP-II/A-Broccoli
ggacggagacggtcgggtcatatgatgatcgcgtggatatggcacgcattgaattgttggacaccgtaaatgtcctaacaatattcgagtagagtgtgggctccgtcc

SEQ ID NO: 32 - DNA, 108 bp, Artificial Sequence
  5HTP-II/U-Broccoli
ggacggagacggtcgggtctatagatgatcgcgtggatatggcacgcattgaattgttggacaccgtaaatgtcctaacatatatcgagtagagtgtgggctccgtcc

SEQ ID NO: 33 - DNA, 110 bp, Artificial Sequence
  5HTP-IV/A-Broccoli
ggacggagacggtcgggtcatattctggttcgcgtagatatggcacgcaattgaagaatgggcaccgtaaatgtctgtagacatattcgagtagagtgtgggctccgtcc

SEQ ID NO: 34 - DNA, 110 bp, Artificial Sequence
  5HTP-IV/U-Broccoli
ggacggagacggtcgggtctatatctggttcgcgtagatatggcacgcaattgaagaatgggcaccgtaaatgtctgtagactatatcgagtagagtgtgggctccgtcc

SEQ ID NO: 35 - DNA, 129 bp, Artificial Sequence
  5HTP-VII/A-Broccoli
ggacggagacggtcgggtcatatatattcgatgtaatctccaaaccattcgaaagagtgggacgctagacctccggcctaaaccgaaaggtaggtagcggggctaggtagtagagtgtgggctccgtcc

SEQ ID NO: 36 - DNA, 129 bp, Artificial Sequence
  5HTP-VII/U-Broccoli
ggacggagacggtcgggtctatatgtaatctccaaaccattcgaaagagtgggacgctagacctccggcctaaaccgaaaggtaggtagcggggctaggtatatatcgagtagagtgtgggctccgtcc

SEQ ID NO: 37 - DNA, 108 bp, Artificial Sequence
  5HTP-VIII/A-Broccoli
ggacggagacggtcgggtcatattgtttggtatccaatgaaaatgtactaccaacttgaatctcccaaataggctaggtaatattcgagtagagtgtgggctccgtcc

SEQ ID NO: 38 - DNA, 108 bp, Artificial Sequence
  5HTP-VIII/U-Broccoli
ggacggagacggtcgggtctatatgtttggtatccaatgaaaatgtactaccaacttgaatctcccaaataggctaggtatatatcgagtagagtgtgggctccgtcc

SEQ ID NO: 39 - DNA, 221 bp, Artificial Sequence
  5HTP-IV/pbuE riboswitch
aatattgagctgttgacaattaatcatcggctcgtataatgtgtggaattaaatagctattatcaggatttttctggttcgcgtagatatggcacgcaattgaagaatgggcaccgtaaatgtctgtagacaaaatcctgattacaaaatttgtttatgacattttttgtaatcaggatttttttatttatcaaaacatttaagtaaaggagtttgttatg

SEQ ID NO: 40 - DNA, 52 bp, Artificial Sequence
  Outer primer
ggccgtaatacgactcactataggagcccggatagctcagtcggtagagcag

SEQ ID NO: 41 - DNA, 30 bp, Artificial Sequence
  Outer primer
tggcgcccgaacagggacttgaaccctgga

SEQ ID NO: 42 - RNA, 61 nt, Artificial Sequence
  GR scaffold is derived from the aptamer domain of the B. subtilis xpt-pbuX guanine riboswitch
cauauaaucgcguggauauggcacgcaaguuucuaccgggcaccguaaauguccgacuaug

SEQ ID NO: 43 - RNA, 82 nt, Artificial Sequence
  CDG scaffold derived from the aptamer domain V. cholera Vc2 cyclic di-GMP riboswitch (PDB ID 3IWN)
cacgcacagggcaaaccauucgaaagagugggacgcaaagccuccggccuaaaccgaaagguagguagcgggguuaccgaug

SEQ ID NO: 44 - RNA, 62 nt, Artificial Sequence
  HH scaffold derived from the S. mansoni hammerhead ribozyme (PDB ID 3ZP8)
gcguccugguauccaaugaaaauguacuaccagcugaugagucccaaauaggacgaaaacgc

SEQ ID NO: 45 - RNA, 63 nt, Artificial Sequence
  5HTP-I
cucuuaacgccgcguggauaugcacgcaaccgugaaucgggcaccguaaauuccguaaguggg

SEQ ID NO: 46 - RNA, 65 nt, Artificial Sequence
  5HTP-II
cucugaugaucgcguggauauggcacgcauugaauuguuggrcaccguaaauguccuaacacggg

SEQ ID NO: 47 - RNA, 15 nt, Artificial Sequence
  5HTP-III
ggauauggcacgcua

SEQ ID NO: 48 - RNA, 13 nt, Artificial Sequence
  5HTP-III  (Trp aptamer)
gaccguaaugucc

SEQ ID NO: 49 - RNA, 63 nt, Artificial Sequence
  5HTP-IV
  misc_feature (3)..(4): n is a, c, g, or u
  misc_feature (6)..(9): n is a, c, g, or u
ucnnynnnncgcgurgauauggcacgcaauugaagaaugggcaccguaaaugucuguagacgg

SEQ ID NO: 50 - RNA, 63 nt, Artificial Sequence
  5HTP-V
  misc_feature (4)..(4): n is a, c, g, or u
  misc_feature (6)..(6): n is a, c, g, or u
ucrnynrrycgcguggauauggcacgcaggagauguguggacaccguaaauguccguaggcgg

SEQ ID NO: 51 - RNA, 63 nt, Artificial Sequence
  5HTP-VI
  misc_feature (6)..(8): n is a, c, g, or u
  misc_feature (29)..(32): n is a, c, g, or u
  misc_feature (37)..(37): n is a, c, g, or u
ucaacnnnycgcguggauauggcacgcannnnucyanugggcaccguaaauguccguagacgg

SEQ ID NO: 52 - RNA, 139 nt, Artificial Sequence
  Secondary structure of an artificial 5HTP/serotonin “ON” riboswitch based upon 5HTP-IV aptamer
aauuaaauagcuauuaucaggauuuuucugguucgcguagauauggcacgcaauugaagaaugggcaccguaaaugucuguagacaaaauccugauuacaaaauuuguuuaugacauuuuuuguaaucaggauuuuuuu

SEQ ID NO: 53 - RNA, 82 nt, Artificial Sequence
  5HTP-VII
  misc_feature (38)..(40): n is a, c, g, or u
  misc_feature (71)..(71): n is a, c, g, or u
cauguaaucuccaaaccauucgaaagagugggacgyynnnccuccggccuaaaccgaaagguagguagcgnggcyaggurug

SEQ ID NO: 54 - RNA, 61 nt, Artificial Sequence
  5HTP-VIII
  misc_feature (33)..(33): n is a, c, g, or u
  misc_feature (35)..(37): n is a, c, g, or u
gcuguyugguauccaaugaaaauguacuaccanynnnraucucccaaauaggcuaggurgc

SEQ ID NO: 55 - RNA, 61 nt, Artificial Sequence; identical to SEQ ID NO: 54
  5HTP-VIII
  misc_feature (33)..(33): n is a, c, g, or u
  misc_feature (35)..(37): n is a, c, g, or u

SEQ ID NO: 56 - RNA, 56 nt, Artificial Sequence
  5HTP-II-Broccoli biosensors
gaugaucgcguggauauggcacgcuugaauuguuggacaccguaaauguccuaaca

SEQ ID NO: 57 - RNA, 58 nt, Artificial Sequence
  5HTP-IV -Broccoli biosensors
ucugguucgcguagauauggcacgcaauugaagaaugggcaccguaaaugucuguaga

SEQ ID NO: 58 - RNA, 78 nt, Artificial Sequence
  5HTP-VII-Broccoli biosensors
uguaaucuccaaaccauucgaaagagugggacgcuagaccuccggccuaaaccgaaagguagguagcggggcuaggua

SEQ ID NO: 59 - RNA, 57 nt, Artificial Sequence
  5HTP-VIII -Broccoli biosensors
uguuugguauccaaugaaaauguacuaccaacuugaaucucccaaauaggcuaggua

SEQ ID NO: 60 - RNA, 19 nt, Artificial Sequence
  Broccoli
ggacggagacggucggguc

SEQ ID NO: 61 - RNA, 24 nt, Artificial Sequence
  Broccoli
ucgaguagagugugggcuccgucc

SEQ ID NO: 62 - RNA, 120 nt, Artificial Sequence
  Guanine riboswitch aptamer (GR) RNA library for the selection using the SuperScript III RT
  misc_feature (27)..(33): n is a, c, g, or u
  misc_feature (53)..(62): n is a, c, g, or u
  misc_feature (80)..(85): n is a, c, g, or u
ggacuucgguccaagcuaaugcacucnnnnnnncgcguggauauggcacgcannnnnnnnnngggcaccguaaauguccnnnnnngggugcauuagcaaaaucgggcuucgguccgguuc

SEQ ID NO: 63 - RNA, 118 nt, Artificial Sequence
  GR RNA library used for the selection using the GsI-IIC RT
  misc_feature (26)..(32): n is a, c, g, or u
  misc_feature (52)..(61): n is a, c, g, or u
  misc_feature (79)..(84): n is a, c, g, or u
ggacuucgguccuuggauaggacucnnnnnnncgcguggauauggcacgcannnnnnnnnngggcaccguaaauguccnnnnnnggguccuauccccaaucgggcuucgguccgguuc

SEQ ID NO: 64 - RNA, 135 nt, Artificial Sequence
  di-cyclic GMP riboswitch aptamer (CG
  misc_feature (25)..(33): n is a, c, g, or u
  misc_feature (58)..(62): n is a, c, g, or u
  misc_feature (96)..(102): n is a, c, g, or u
ggacuucgguccuuggauaggacannnnnnnnncaaaccauucgaaagagugggacgnnnnnccuccggccuaaaccgaaagguagguagcggggnnnnnnnuguccuauccccaaucgggcuucgguccgguuc

SEQ ID NO: 65 - RNA, 115 nt, Artificial Sequence
  Hammerhead ribozyme (HR) library
  misc_feature (25)..(28): n is a, c, g, or u
  misc_feature (55)..(64): n is a, c, g, or u
  misc_feature (75)..(82): n is a, c, g, or u
ggacuucgguccuuggauaggagcnnnnugguauccaaugaaaauguacuaccannnnnnnnnncccaaauaggnnnnnnnngcuccuauccccaaucgggcuucgguccgguuc

SEQ ID NO: 66 - RNA, 82 nt, Artificial Sequence
  DGR-I
  misc_feature (40)..(40): n is a, c, g, or u
ggauaggacuccguaauucgcguggauauggcacgcagunagguguugggcaccguaaaugcyccayruagcuauccccaau

SEQ ID NO: 67 - RNA, 80 nt, Artificial Sequence
  DGR-II
ggauaggacucgyuaagacgcguggauauggcacgcaauuaaguguugggcaccguaaauguccuccggcuayccccaau

SEQ ID NO: 68 - RNA, 86 nt, Artificial Sequence
  DGR-III
  misc_feature (38)..(43): n is a, c, g, or u
  misc_feature (69)..(69): n is a, c, g, or u
ggauaggacucaggugggcgcguggauauggcacgcannnnnngugggggcaccguaaauguccgagunrggruccuauccccaau

SEQ ID NO: 69 - RNA, 86 nt, Artificial Sequence
  DGR-IV
  misc_feature (38)..(42): n is a, c, g, or u
  misc_feature (75)..(75): n is a, c, g, or u
ggauaggacucagguggucgcguggauauggcacgcannnnnrrggugggcaccguaaauguccgggurrggguncuauccccaau

SEQ ID NO: 70 - DNA, 74 bp, Artificial Sequence; identical to SEQ ID NO: 14
  5GR-II

SEQ ID NO: 71 - DNA, 84 bp, Artificial Sequence; identical to SEQ ID NO: 15
  5GR-I aptamer

SEQ ID NO: 72 - DNA, 73 bp, Artificial Sequence; identical to SEQ ID NO: 16
  5GR-II aptamer

SEQ ID NO: 73 - DNA, 81 bp, Artificial Sequence; identical to SEQ ID NO: 17
  5GR-III aptamer

SEQ ID NO: 74 - DNA, 81 bp, Artificial Sequence; identical to SEQ ID NO: 18
  5GR-IV aptamer

SEQ ID NO: 75 - DNA, 81 bp, Artificial Sequence; identical to SEQ ID NO: 19
  5GR-V aptamer

SEQ ID NO: 76 - DNA, 81 bp, Artificial Sequence; identical to SEQ ID NO: 20
  5GR-VI aptamer

SEQ ID NO: 77 - DNA, 98 bp, Artificial Sequence; identical to SEQ ID NO: 21
  5GR-VII aptamer

SEQ ID NO: 78 - DNA, 77 bp, Artificial Sequence; identical to SEQ ID NO: 22
  5GR-VIII aptamer

SEQ ID NO: 79 - DNA, 140 bp, Artificial Sequence; identical to SEQ ID NO: 23
  5GR-I, SHAPE

SEQ ID NO: 80 - DNA, 142 bp, Artificial Sequence; identical to SEQ ID NO: 24
  5GR-II, SHAPE

SEQ ID NO: 81 - DNA, 139 bp, Artificial Sequence; identical to SEQ ID NO: 25
  5GR-III, SHAPE

SEQ ID NO: 82 - DNA, 116 bp, Artificial Sequence; identical to SEQ ID NO: 26
  5GR-IV, SHAPE

SEQ ID NO: 83 - DNA, 116 bp, Artificial Sequence; identical to SEQ ID NO: 27
  5GR-V, SHAPE

SEQ ID NO: 84 - DNA, 116 bp, Artificial Sequence; identical to SEQ ID NO: 28
  5GR-VI, SHAPE

SEQ ID NO: 85 - DNA, 133 bp, Artificial Sequence; identical to SEQ ID NO: 29
  5CG-I, SHAPE

SEQ ID NO: 86 - DNA, 112 bp, Artificial Sequence; identical to SEQ ID NO: 30
  5HR-I, SHAPE

SEQ ID NO: 87 - DNA, 159 bp, Artificial Sequence
  5GR-II/CM-2
gcccggatagctcagtcggtagagcagcggagacggtcgggtctagatgatcgcgtggatatggcacgcattgaattgttggacaccgtaaatgtcctaacatatcgagtagagtgtgggctccgcgggtccagggttcaagtccctgttcgggcgcca

SEQ ID NO: 88 - DNA, 161 bp, Artificial Sequence
  5GR-II/CM-3
gcccggatagctcagtcggtagcgcagcggagacggtcgggtctatgatgatcgcgtggatatggcacgcattgaattgttggacaccgtaaatgtcctaacaatatcgagtagagtgtgggctccgcgggtccagggttcaagtccctgttcgggcgcca

SEQ ID NO: 89 - DNA, 163 bp, Artificial Sequence
  5GR-II/CM-4
gcccggatagctcagtcggtagagcagcggagacggtcgggtctatagctgatcgcgtggatatggcacgcattgaattgttggacaccgtaaatgtcctaacatatatcgagtagagtgtgggctccgcgggtccagggttcaagtccctgttcgggcgcca

SEQ ID NO: 90 - DNA, 165 bp, Artificial Sequence
  5GR-II/CM-5
gcccggatagctcagtcggtagagcagcggagacggtcgggtctatatgatgatcgcgtggatatggcacgcattgaattgttggacaccgtaaatgtcctaacaatatatcgagtagagtgtgggctccgcgggtccagggttcaagtccctgttcgggcgcca

SEQ ID NO: 91 - DNA, 161 bp, Artificial Sequence
  5GR-IV/CM-2
gcccggatagctcagtcggtagagcagcggagacggtcgggtctatctggttcgcgtagatatggcacgcaattgaagaatgggcaccgtaaatgtctgtagactatcgagtagagtgtgggctccgcgggtccagggttcaagtccctgttcgggcgcca

SEQ ID NO: 92 - DNA, 163 bp, Artificial Sequence
  5GR-IV/CM-3
gcccggatagctcagtcggtagagcagcggagacggtcgggtctattctggttcgcgtagatatggcacgcaattgaagaatgggcaccgtaaatgtctgtagacatatcgagtagagtgtgggctccgcgggtccagggttcaagtccctgttcgggcgcca

SEQ ID NO: 93 - DNA, 165 bp, Artificial Sequence
  5GR-IV/CM-4
gcccggatagctcagtcggtagagcagcggagacggtcgggtctatatctggttcgcgtagatatggcacgcaattgaagaatgggcaccgtaaatgtctgtagactatatcgagtagagtgtgggctccgcgggtccagggttcaagtccctgttcgggcgcca

SEQ ID NO: 94 - DNA, 167 bp, Artificial Sequence
  5GR-IV/CM-5
gcccggatagctcagtcggtagagcagcggagacggtcgggtctatattctggttcgcgtagatatggcacgcaattgaagaatgggcaccgtaaatgtctgtagacatatatcgagtagagtgtgggctccgcgggtccagggttcaagtccctgttcgggcgcca

SEQ ID NO: 95 - DNA, 161 bp, Artificial Sequence
  5GR-V/CM-2
gcccggatagctcagtcggtagagcagcggagacggtcgggtctaattcggccgcgtggatatggcacgcaggagatgtgtggacaccgtaaatgtccgtaggctatcgagtagagtgtgggctccgcgggtccagggttcaagtccctgttcgggcgcca

SEQ ID NO: 96 - DNA, 163 bp, Artificial Sequence
  5GR-V/CM-3
gcccggatagctcagtcggtagagcagcggagacggtcgggtctatattcggccgcgtggatatggcacgcaggagatgtgtggacaccgtaaatgtccgtaggcatatcgagtagagtgtgggctccgcgggtccagggttcaagtccctgttcgggcgcca

SEQ ID NO: 97 - DNA, 165 bp, Artificial Sequence
  5GR-V/CM-4
gcccggatagctcagtcggtagagcagcggagacggtcgggtctataattcggccgcgtggatatggcacgcaggagatgtgtggacaccgtaaatgtccgtaggctatatcgagtagagtgtgggctccgcgggtccagggttcaagtccctgttcgggcgcca

SEQ ID NO: 98 - DNA, 167 bp, Artificial Sequence
  5GR-V/CM-5
gcccggatagctcagtccgtagagcagcggagacggtcgggtctatatattcggccgcgtggatatggcacgcaggagatgtgtggacaccgtaaatgtccgtaggcatatatcgagtagagtgtgggctccgcgggtccagggttcaagtccctgttcgggcgcca

SEQ ID NO: 99 - DNA, 161 bp, Artificial Sequence
  5GR-VI/CM-2
gcccggatagctcagtcggtagagcagcggagacggtcgggtctaaacatctcgcgtggatatggcacgcagacttccagtgggcaccgtaaatgtccgtagactatcgagtagagtgtgggctccgcgggtccagggttcaagtccctgttcgggcgcca

SEQ ID NO: 100 - DNA, 163 bp, Artificial Sequence
  DGR-VI/CM-3
gcccggatagctcagtcggtagagcagcggagacggtcgggtctataacatctcgcgtggatatggcacgcagacttccagtgggcaccgtaaatgtccgtagacatatcgagtagagtgtgggctccgcgggtccagggttcaagtccctgttcgggcgcca

SEQ ID NO: 101 - DNA, 165 bp, Artificial Sequence
  DGR-VI/CM-4
gcccggatagctcagtcggtagagcagcggagacggtcgggtctataaacatctcgcgtggatatggcacgcagacttccagtgggcaccgtaaatgtccgtagactatatcgagtagagtgtgggctccgcgggtccagggttcaagtccctgttcgggcgcca

SEQ ID NO: 102 - DNA, 167 bp, Artificial Sequence
  DGR-VI/CM-5
gcccggatagctcagtcggtagagcagcggagacggtcgggtctatataacatctcgcgtggatatggcacgcagacttccagtgggcaccgtaaatgtccgtagacatatatcgagtagagtgtgggctccgcgggtccagggttcaagtccctgttcgggcgcca

SEQ ID NO: 103 - DNA, 167 bp, Artificial Sequence
  DGR-I/CM-2
gcccggatagctcagtcggtagagcagcggagacggtcgggtctagactccgtaattcgcgtggatatggcacgcagtcaggtgttgggcaccgtaaatgtcccacatagtatcgagtagagtgtgggctccgcgggtccagggttcaagtccctgttcgggcgcca

SEQ ID NO: 104 - DNA, 169 bp, Artificial Sequence
  DGR-I/CM-3
gcccggatagctcagtcggtagagcagcggagacggtcgggtctatgactccgtaattcgcgtggatatggcacgcagtcaggtgttgggcaccgtaaatgtcccacatagatatcgagtagagtgtgggctccgcgggtccagggttcaagtccctgttcgggcgcca

SEQ ID NO: 105 - DNA, 171 bp, Artificial Sequence
  DGR-I/CM-4
gcccggatagctcagtcggtagagcagcggagacggtcgggtctatagactccgtaattcgcgtggatatggcacgcagtcaggtgttgggcaccgtaaatgtcccacatagtatatcgagtagagtgtgggctccgcgggtccagggttcaagtccctgttcgggcgcca

SEQ ID NO: 106 - DNA, 173 bp, Artificial Sequence
  DGR-I/CM-5
gcccggatagctcagtcggtagagcagcggagacggtcgggtctatatgactccgtaattcgcgtggatatggcacgcagtcaggtgttgggcaccgtaaatgtcccacatagatatatcgagtagagtgtgggctccgcgggtccagggttcaagtccctgttcgggcgcca

SEQ ID NO: 107 - DNA, 166 bp, Artificial Sequence
  DGR-II/CM-2
gcccggatagctcagtcggtagagcagcggagacggtcgggtctagactccgtaattcgcgtggatatggcacgcagtaaggtattgggcaccgtaaatgtccatatgttatcgagtagagtgtgggctccgcgggtccagggttcaagtccctgttcgggcgcca

SEQ ID NO: 108 - DNA, 168 bp, Artificial Sequence
  DGR-II/CM-3
gcccggatagctcagtcggtagagcagcggagacggtcgggtctatgactccgtaattcgcgtggatatggcacgcagtaaggtattgggcaccgtaaatgtccatatgtatatcgagtagagtgtgggctccgcgggtccagggttcaagtccctgttcgggcgcca

SEQ ID NO: 109 - DNA, 170 bp, Artificial Sequence
  DGR-II/CM-4
gcccggatagctcagtcggtagagcagcggagacggtcgggtctatagactccgtaattcgcgtggatatggcacgcagtaaggtattgggcaccgtaaatgtccatatgttatatcgagtagagtgtgggctccgcgggtccagggttcaagtccctgttcgggcgcca

SEQ ID NO: 110 - DNA, 172 bp, Artificial Sequence
  DGR-II/CM-5
gcccggatagctcagtcggtagagcagcggagacggtcgggtctatatgactccgtaattcgcgtggatatggcacgcagtaaggtattgggcaccgtaaatgtccatatgtatatatcgagtagagtgtgggctccgcgggtccagggttcaagtccctgttcgggcgcca

SEQ ID NO: 111 - DNA, 161 bp, Artificial Sequence
  DGR-III/CM-2
gcccggatagctcagtcggtagagcagcggagacggtcgggtctaaggtgggcgcgtggatatggcacgcatgtttggtgggggcaccgtaaatgtccgagttatatcgagtagagtgtgggctccgcgggtccagggttcaagtccctgttcgggcgcca

SEQ ID NO: 112 - DNA, 163 bp, Artificial Sequence
  DGR-III/CM-3
gcccggatagctcagtcggtagagcagcggagacggtcgggtctataggtgggcgcgtggatatggcacgcatgtttggtgggggcaccgtaaatgtccgagttaatatcgagtagagtgtgggctccgcgggtccagggttcaagtccctgttcgggcgcca

SEQ ID NO: 113 - DNA, 165 bp, Artificial Sequence
  DGR-III/CM-4
gcccggatagctcagtcggtagagcagcggagacggtcgggtctataaggtgggcgcgtggatatggcacgcatgtttggtgggggcaccgtaaatgtccgagttatatatcgagtagagtgtgggctccgcgggtccagggttcaagtccctgttcgggcgcca

SEQ ID NO: 114 - DNA, 167 bp, Artificial Sequence
  DGR-III/CM-5
gcccggatagctcagtcggtagagcagcggagacggtcgggtctatataggtgggcgcgtggatatggcacgcatgtttggtgggggcaccgtaaatgtccgagttaatatatcgagtagagtgtgggctccgcgggtccagggttcaagtccctgttcgggcgcca

SEQ ID NO: 115 - DNA, 161 bp, Artificial Sequence
  DGR-IV/CM-2
gcccggatagctcagtcggtagagcagcggagacggtcgggtctaaggtggtcgcgtggatatggcacgcataatagaggtgggcaccgtaaatgtccgggtaatatcgagtagagtgtgggctccgcgggtccagggttcaagtccctgttcgggcgcca

SEQ ID NO: 116 - DNA, 163 bp, Artificial Sequence
  DGR-IV/CM-3
gcccggatagctcagtcggtagagcagcggagacggtcgggtctataggtggtcgcgtggatatggcacgcataatagaggtgggcaccgtaaatgtccgggtaaatatcgagtagagtgtgggctccgcgggtccagggttcaagtccctgttcgggcgcca

SEQ ID NO: 117 - DNA, 165 bp, Artificial Sequence
  DGR-IV/CM-4
gcccggatagctcagtcggtagagcagcggagacggtcgggtctataaggtggtcgcgtggatatggcacgcataatagaggtgggcaccgtaaatgtccgggtaatatatcgagtagagtgtgggctccgcgggtccagggttcaagtccctgttcgggcgcca

SEQ ID NO: 118 - DNA, 167 bp, Artificial Sequence
  DGR-IV/CM-5
gcccggatagctcagtcggtagagcagcggagacggtcgggtctatataggtggtcgcgtggatatggcacgcataatagaggtgggcaccgtaaatgtccgggtaaatatatcgagtagagtgtgggctccgcgggtccagggttcaagtccctgttcgggcgcca